(19) United States
(12) Patent Application Publication
(10) Pub. No.: US 2024/0382451 A1
(43) Pub. Date: Nov. 21, 2024

(54) ORGANONITRO AND SULFOXYALKYL ORGANONITRO COMPOUNDS FOR USE IN TREATING MEDICAL DISORDERS

(71) Applicant: EpicentRx, Inc., La Jolla, CA (US)

(72) Inventors: Bryan T. ORONSKY, Los Altos Hills, CA (US); Scott CAROEN, San Francisco, CA (US); Tony R. REID, San Diego, CA (US)

(21) Appl. No.: 18/570,439

(22) PCT Filed: Jun. 16, 2022
(86) PCT No.: PCT/US2022/033856

§ 371 Date: Dec. 14, 2023

Related U.S. Application Data
(60) Provisional application No. 63/211,329, filed on Jun. 16, 2021.
(60) Provisional application No. 63/225,249, filed on Jul. 23, 2021.

(51) Int. Cl. A61K 31/397; A61K 45/06; A61P 1/00; A61P 25/16; A61P 25/28; A61P 39/00

(52) U.S. Cl. CPC A61K 31/397; A61K 45/06; A61P 1/00; A61P 25/16; A61P 25/28; A61P 39/00

(57) ABSTRACT

Provided herein are methods of treating and preventing a disorder selected from the group consisting of an autoimmune disorder, inflammatory disorder, immune disorder, bone and joint disorder, neurodegenerative disorder, and neuromuscular disorder in a. subject in need thereof. The method includes administering an effective amount of an organonitro or sulfoxyalkyl organonitro compound to the subject, which may include using a dosing regimen that comprises administering loading and maintenance doses.

### CROSS-REFERENCE TO RELATED APPLICATIONS

This application claims priority to and the benefit of U.S. Provisional Application No. 63/211,329, filed on Jun. 16, 2021, and 63/225,249, filed on Jul. 23, 2021, the disclosures of which are incorporated herein by reference in their entirety.

### FIELD OF THE INVENTION

The invention provides methods of using organonitro and sulfoxyalkyl organonitro and related compounds and compositions containing such compounds to treat medical disorders, such as an autoimmune disorder, inflammatory disorder, metabolic disorder, bone and joint disorder, neurodegenerative disorder, or neuromuscular disorder in a subject.

### BACKGROUND

A common feature found in many human diseases is inflammation. This is particularly true in the case of autoimmune diseases, which result from the loss of self-tolerance and the mounting of a specific immune response against “self” molecules or antigens, which drives inflammation that is typically chronic. Medical management of autoimmune diseases (e.g., rheumatoid arthritis (RA), sarcoidosis, systemic lupus erythematosus (SLE), Huntington's disease, end stage renal disease, systemic sclerosis or scleroderma, myositis, diabetes type 1, multiple sclerosis, Sjögren's syndrome, psoriasis, primary biliary cirrhosis, autoimmune hepatitis, Graves' disease, Addison's disease, tuberculosis, Crohn's disease, ulcerative colitis, inflammatory bowel disease, autoimmune hemolytic anemia, idiopathic thrombocytopenic purpura and idiopathic neutropenia, and coeliac (also known as celiac) disease) involves the use of anti-inflammatory medications. Anti-inflammatory medications include, for example, non-steroidal anti-inflammatory agents (NSAIDS), glucocorticoids, biologics that neutralize key proinflammatory cytokines or interfere with lymphocytes themselves, and synthetic signaling pathway inhibitors.

Although methods of treatment for such diseases and disorders are available, current management options rarely lead to cure or drug-free remission, and immunosuppressant drugs often downregulate the normal adaptive immune system. Several of the therapies that are in use today are associated with an enhanced risk of serious and opportunistic infections, teratogenicity, and the development of malignancy. This is in addition to disease comorbidities and drug-specific side-effects associated with the administration of, for example, chronic glucocorticoids and NSAIDS.

Other diseases associated with inflammation include metabolic disorders, particularly gout, type 2 diabetes, atherosclerosis, fatty liver disease, stroke and cardiovascular disease; and neurodegenerative and neuromuscular disorders such as Alzheimer's Disease, Parkinson's Disease, Amyotrophic Lateral Sclerosis. Myasthenia Gravis, Multifocal Motor Neuropathy, Primary Lateral Sclerosis, Spinal Muscular Atrophy, Kennedy's Disease, and Spinocerebellar Ataxia.

Despite the efforts made to date, there is an ongoing and unmet need for safe and effective treatment options for diseases and disorders associated with inflammation, including autoimmune disorders, inflammatory disorders, neurodegenerative disorders, and neuromuscular disorder.

### SUMMARY

The present disclosure provides methods of treating a disorder selected from the group consisting of an autoimmune disorder, inflammatory disorder, neurodegenerative disorder, and neuromuscular disorder in a subject in need thereof. The method comprises administering to the subject an organonitro or sulfoxyalkyl organonitro compound by way of a loading dose and a maintenance dose. The disclosure also provides methods for prophylaxis/prevention of the above-described disorders in a subject in need thereof. The methods comprise administering to the subject an organonitro or sulfoxyalkyl organonitro compound, which optionally can be administered via a dosing regimen that involves the administration of a loading dose and a maintenance dose. The disclosure further provides pharmaceutical compositions comprising an organonitro or sulfoxyalkyl organonitro compound, a second therapeutic agent, and one or more pharmaceutically acceptable excipients.

In one aspect, the disclosure provides a method of treating a disorder selected from the group consisting of an autoimmune disorder, inflammatory disorder, neurodegenerative disorder, and neuromuscular disorder in a subject in need thereof. The method comprising administering a loading dose of RRx-001 or an analog thereof to the subject in an amount effective to ameliorate a symptom of the disorder, and thereafter administering a maintenance dose of RRx-001 or the analog thereof to maintain the amelioration of the symptom for a prolonged period of time.

In various embodiments of the disclosure, the loading dose comprises at least 1 mg. In various embodiments of the disclosure, the loading dose comprises at least 1 mg/m².

In various embodiments of the disclosure, the loading dose comprises multiple doses administered after the first day with a periodic interval of at least 3, 4, 5, 6, 7, 14, 21, or 28 days separating each dose to achieve amelioration of one or more symptoms of the disorder. In various embodiments of the disclosure, the doses administered after the first day are the same as the dose administered on the first day.

In various embodiments of the disclosure, the maintenance dose is administered after the loading dose with a periodic interval of about 7, 14, 21, or 28 days separating each dose to maintain amelioration of one or more symptoms of the disorder.

In various embodiments of the disclosure, the maintenance dose is less than the loading dose.

In various embodiments of the disclosure, the prolonged period of time is at least 1 month, or 3, 6, 9, or 12 months.

In another aspect, the disclosure provides a method of preventing the initiation, development, or worsening of a symptom of any of the above-described disorders in a subject in need thereof. The method comprises administering an effective amount of RRx-001 or an analog thereof to the subject to prevent the initiation, development or worsening of the symptom of the disorder.

In certain embodiments the disorder is a neurodegenerative disorder. Exemplary disorders include, for example, Alzheimer's disease and other dementias, Parkinson's disease and PD-related disorders, multiple system atrophy, prion diseases, motor neuron diseases (MND), Huntington's disease, spinocerebellar ataxia, spinal muscular atrophy, Batten disease, Freidreich's ataxia, vascular dementia, transactive response DNA-binding protein-43, proteinopathies, incurable neurodegenerative diseases with pediatric onset, purine and pyrimidine defects, metal metabolism such as Wilson disease, pantothenate kinase associated neurodegeneration and neurodegeneration with brain iron accumulation, leukodystrophy, peroxisomal disorders, lysosomal storage disorders, congenital disorders of glycosylation, creatine disorders, and Rasmussen's encephalitis.

In various embodiments of the disclosure, the effective amount comprises at least 1 mg. In various embodiments of the disclosure, the effective amount comprises at least 1 mg/m².

In various embodiments of the disclosure, the RRx-001 or analog thereof is administered to the subject periodically.

In various embodiments of the disclosure the RRx-001 or analog thereof is combined with blood prior to administration to the subject. The blood can be blood harvested from the subject or blood harvested from a donor that matches the blood type of the subject.

In various embodiments of the disclosure, the methods described herein further comprise administering a second, different agent to the subject.

In various embodiments of the disclosure, the subject is a human.

In another aspect, provided herein are pharmaceutical compositions comprising RRx-001 or an analog thereof and at least one additional active component selected from the group consisting group of an anti-inflammatory agent, a neuroprotective agent, a corticosteroid, an immunosuppressant, and a disease-modifying anti-rheumatic drug (DMARD), and one or more pharmaceutically acceptable excipients.

In some aspect, provided herein are methods of treating a disorder or condition associated with interleukin 1 beta (IL-1β) or interleukin 18 (IL-18) expression or release. In some embodiments, the method comprises administering an effective amount of RRx-001 or an analog thereof to ameliorate a symptom of the disorder or condition. In some embodiments, the disorder or condition is an autoimmune disorder or an inflammatory disorder. In some embodiments, the disorder or condition is pediatric fever syndrome, acne vulgaris, hidradenitis suppurativa, psoriasis, rheumatoid, systemic juvenile idiopathic arthritis sepsis, chronic inflammation, aging, viral infection, asthma, congestive heart failure, angina, liver steatosis, diabetes, familial cold autoinflammatory syndrome (FCAS), Muckle-Wells syndrome (MWS), neonatal-onset multisystem inflammatory disease (NOMID), chronic infantile neurologic, cutaneous, arthritis (CINCA) syndrome, or cerebral edema due to intracerebral hemorrhage. In some embodiments, administering an effective amount of RRx-001 or an analog thereof does not lead to severe infection or end organ toxicity.

In some embodiments, the method further comprises administering a second agent. In some embodiments, the second agent comprises an IL-1α inhibitor, an IL-1β inhibitor, an IL-18 inhibitor, or an inflammasome inhibitor, or any combination thereof. In some embodiments, the second agent comprises canakinumab, anakinra, rilonacept, resveratrol, curcumin, MABp1, GSK-1070806 antibody, MCC950, β-hydroxybutyrate, glibenclamide, oridonin, JC121, JC124, YQ128, apigenin, cardamonin, Isoliquiritigenin, Glycyrrhizin, Luteolin, Quercetin, Artemisia, Caffeic acid phenethyl ester, Obovatol, Parthenolide, Shikonin, Sulforaphane, Tranilast, OLT1177 (Dapansutrile), CY-09, Methylene Blue, Disulfuram (Antabuse), Fenamic acid derivatives, Fluoxetine, β-Hydroxybutyrate (BHB), BAY 11-7082 (BAY), or X-11-5-27, or any combination thereof. In some embodiments, a side effect associated with the second agent is reduced.

In some aspect, provided herein are methods of controlling activity of a mononuclear phagocyte. In some embodiments, the method comprises exposing the mononuclear phagocyte to an effective amount of RRx-001 or an analog thereof. In some embodiments, the activity of the mononuclear phagocyte is expression or release of interleukin 1 beta (IL-1β) from the mononuclear phagocyte. In some embodiments, controlling activity of the mononuclear phagocyte is reducing the expression or release of interleukin 1 beta (IL-1β) from the mononuclear phagocyte. In some embodiments, the mononuclear phagocyte is a macrophage or a dendritic cell, or a combination thereof. In some embodiments, the exposure occurs ex vivo. In some embodiments, the mononuclear phagocyte is collected from a human subject. In some embodiments, the mononuclear phagocyte exposed to the effective amount of RRx-001 or an analog thereof is re-introduced into the human subject. In some embodiments, the mononuclear phagocyte exposed to the effective amount of RRx-001 or an analog thereof is re-introduced into the human subject by infusion. In some embodiments, immunological tolerance is increased in the human subject after the mononuclear phagocyte is re-introduced.

### BRIEF DESCRIPTION OF FIGURES

FIG. 1A is a plot comparing the latency in reaching the exact hidden platform location in a Morris water maze test of three groups of mice (triple transgenic Alzheimer's disease (AD) mouse model (3×Tg-AD) receiving 2 mg/kg RRx-001; 3×Tg-AD receiving vehicle; and non-transgenic control mice receiving no treatment) after 3 months of treatment, with the RRx-001- and vehicle-treated mice receiving treatment once weekly, with treatment occurring through 18-21 months of age.

FIG. 1B is a plot comparing the number of crossings of the platform target quadrant in the Morris water maze test described in FIG. 1A, of the three groups described in FIG. 1A.

FIG. 1C is a plot comparing the trend in latency across multiple trials in reaching the exact hidden platform location of the Morris water maze test platform described in FIG. 1A, of the three groups described in FIG. 1A.

FIG. 2A is a plot comparing the glutathione (GSH) to glutathione disulfide (GSSG) ratios of the three groups described in FIG. 1A after 3 months of treatment.

FIG. 2B is a plot comparing the thiobarbituric acid substance levels of the three groups described in FIG. 1A after 3 months of treatment.

FIG. 2C is a plot comparing the amyloids-β density of the three groups described in FIG. 1A after 3 months of treatment.

FIG. 3A depicts a study scheme for evaluating the effects of RRx-001 on LPS primed mice.

FIG. 3B depicts serum IL-1β levels in mice in three experimental groups measured at baseline, 1 hour after LPS injection, and 3 hours after LPS injection.

FIG. 3C depicts serum IL-18 levels in mice in three experimental groups measured at baseline, 1 hour after LPS injection, and 3 hours after LPS injection.

FIG. 3D depicts immunoblotting of NLRP3 and β-actin from the midbrain of mice in three experimental groups.

FIG. 4A depicts the expression levels of IFN-γ, IL-12, TNF-α, and IL-1β from three experimental groups.

FIG. 4B depicts IL-1β release over time from three experimental groups.

FIG. 4C depicts cytosolic Ca²⁺ levels over time for three experimental groups.

FIG. 5 depicts IL-1β release from four experimental groups of bone marrow derived macrophages (BMDMs).

FIG. 6A depicts changes in mice body weight over a 9-day period for three experimental groups primed with 2% or 4% DSS.

FIG. 6B depicts colon length of mice in three experimental groups primed with 2% or 4% DSS.

FIG. 7A depicts changes in mice body weight over a 9-day period in three experimental groups primed with 2% DSS, where RRx-001 is administered intravascularly.

FIG. 7B depicts changes in mice body weight over a 9-day period in three experimental groups primed with 4% DSS, where RRx-001 is administered intravascularly.

FIG. 7C depicts colon length of mice in three experimental groups primed with 2% DSS, where RRx-001 is administered intravascularly.

FIG. 7D depicts colon length of mice in three experimental groups primed with 4% DSS, where RRx-001 is administered intravascularly.

FIG. 7E depicts serum IL-1β levels in mice in three experimental groups primed with 2% DSS, where RRx-001 is administered intravascularly.

FIG. 7F depicts serum IL-1β levels in mice in three experimental groups primed with 4% DSS, where RRx-001 is administered intravascularly.

FIG. 7G depicts colon homogenate IL-1β levels in mice in three experimental groups primed with 2% DSS, where RRx-001 is administered intravascularly.

FIG. 7H depicts colon homogenate IL-1β levels in mice in three experimental groups primed with 4% DSS, where RRx-001 is administered intravascularly.

FIG. 7I depicts colon homogenate IL-1β levels relative to the average IL-1β level of the control (i.e., DSS+vehicle) in mice in three experimental groups primed with 2% DSS, where RRx-001 is administered intravascularly.

FIG. 7J depicts colon homogenate IL-1β levels relative to the average IL-1β level of the control (i.e., DSS+vehicle) in mice in three experimental groups primed with 4% DSS, where RRx-001 is administered intravascularly.

FIG. 8A depicts changes in mice body weight over a 9-day period in three experimental groups primed with 2% DSS, where RRx-001 is administered intraperitoneally.

FIG. 8B depicts changes in mice body weight over a 9-day period in three experimental groups primed with 4% DSS, where RRx-001 is administered intraperitoneally.

FIG. 8C depicts colon length of mice in three experimental groups primed with 2% DSS, where RRx-001 is administered intraperitoneally.

FIG. 8D depicts colon length of mice in three experimental groups primed with 4% DSS, where RRx-001 is administered intraperitoneally.

FIG. 8E depicts serum IL-1β levels in mice in three experimental groups primed with 2% DSS, where RRx-001 is administered intraperitoneally.

FIG. 8F depicts serum IL-1β levels in mice in three experimental groups primed with 4% DSS, where RRx-001 is administered intraperitoneally.

FIG. 8G depicts colon homogenate IL-1β levels in mice in three experimental groups primed with 2% DSS, where RRx-001 is administered intraperitoneally.

FIG. 8H depicts colon homogenate IL-1β levels in mice in three experimental groups primed with 4% DSS, where RRx-001 is administered intraperitoneally.

### DETAILED DESCRIPTION OF THE INVENTION

The invention provides, in part, methods of using organonitro and sulfoxyalkyl organonitro and related compounds, compositions containing such compounds, to treat medical disorders associated with inflammation, such as autoimmune disorders, inflammatory disorders, neurodegenerative disorders, or neuromuscular disorders in a patient. The practice of the present invention employs, unless otherwise indicated, conventional techniques of organic chemistry, pharmacology, cell biology, and biochemistry. Such techniques are explained in the literature, such as in “Comprehensive Organic Synthesis” (B. M. Trost & I. Fleming, eds., 1991-1992); “Current protocols in molecular biology” (F. M. Ausubel et al., eds., 1987, and periodic updates); and “Current protocols in immunology” (J. E. Coligan et al., eds., 1991), each of which is herein incorporated by reference in its entirety. Various aspects of the invention are set forth below in sections; however, aspects of the invention described in one particular section are not to be limited to any particular section.

### I. Definitions

To facilitate an understanding of the present invention, a number of terms and phrases are defined below.

The terms “a” and “an” as used herein mean “one or more” and include the plural unless the context is inappropriate.

The term “loading dose” as used herein refers to the dose which is administered at the beginning of the treatment regimen, e.g., to ameliorate a symptom of a disorder. The loading dose may be administered as a single dose or in portions over a period of e.g., hours, days, or weeks.

The term “maintenance dose” as used herein refers to a dose that is administered after the loading dose, and may be used to maintain amelioration of a symptom of a disorder. The maintenance dose may be administered in intervals of e.g., days, weeks, or months.

The term “alkyl” as used herein refers to a saturated straight or branched hydrocarbon, such as a straight or branched group of 1-12, 1-10, or 1-6 carbon atoms, referred to herein as C₁-C₁₂alkyl, C₁-C₁₀alkyl, and C₁-C₆alkyl, respectively. Exemplary alkyl groups include, but are not limited to, methyl, ethyl, propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, butyl, isobutyl, t-butyl, pentyl, isopentyl, neopentyl, hexyl, heptyl, octyl, etc.

The term “cycloalkyl” as used herein refers to a saturated cyclic hydrocarbon, such as a cyclic hydrocarbon group of 3-10, or 3-6 carbon atoms, referred to herein as C₃-C₁₀cycloalkyl, and C₃-C₆cycloalkyl, respectively. Exemplary cycloalkyl groups include, but are not limited to, cyclopropyl, cyclobutyl, cyclopentyl, cyclohexyl, etc.

The term “haloalkyl” refers to an alkyl group that is substituted with at least one halogen. For example, —CH₂F, —CHF₂, —CF₃, —CH₂CF₃, —CF₂CF₃, and the like.

The term “aralkyl” refers to an alkyl group substituted with an aryl group.

The term “aryl” is art-recognized and refers to a carbocyclic aromatic group. Representative aryl groups include phenyl, naphthyl, anthracenyl, and the like. Unless specified otherwise, the aromatic ring may be substituted at one or more ring positions with halogen, alkyl, hydroxyl, or alkoxyl. The term “aryl” also includes polycyclic ring systems having two or more carbocyclic rings in which two or more carbons are common to two adjoining rings (the rings are “fused rings”) wherein at least one of the rings is aromatic, e.g., the other cyclic rings may be cycloalkyls, cycloalkenyls, cycloalkynyls, and/or aryls.

The term “heteroaryl” is art-recognized and refers to aromatic groups that include at least one ring heteroatom. In certain instances, a heteroaryl group contains 1, 2, 3, or 4 ring heteroatoms. Representative examples of heteroaryl groups includes pyrrolyl, furanyl, thiophenyl, imidazolyl, oxazolyl, thiazolyl, triazolyl, pyrazolyl, pyridinyl, pyrazinyl, pyridazinyl and pyrimidinyl, and the like. Unless specified otherwise, the heteroaryl ring may be substituted at one or more ring positions with halogen, alkyl, hydroxyl, or alkoxyl. The term “heteroaryl” also includes polycyclic ring systems having two or more rings in which two or more carbons are common to two adjoining rings (the rings are “fused rings”) wherein at least one of the rings is heteroaromatic, e.g., the other cyclic rings may be cycloalkyls, cycloalkenyls, cycloalkynyls, and/or aryls.

The terms ortho, meta and para are art-recognized and refer to 1,2-, 1,3- and 1,4-disubstituted benzenes, respectively. For example, the names 1,2-dimethylbenzene and ortho-dimethylbenzene are synonymous.

As used herein, the term “heterocyclic” represents, for example, an aromatic or nonaromatic ring containing one or more heteroatoms. The heteroatoms can be the same or different from each other. Examples of heteroatoms include, but are not limited to nitrogen, oxygen and sulfur. Aromatic and nonaromatic heterocyclic rings are well-known in the art. Some nonlimiting examples of aromatic heterocyclic rings include pyridine, pyrimidine, indole, purine, quinoline and isoquinoline. Nonlimiting examples of nonaromatic heterocyclic compounds include piperidine, piperazine, morpholine, pyrrolidine and pyrazolidine. Examples of oxygen containing heterocyclic rings include, but are not limited to furan, oxirane, 2H-pyran, 4H-pyran, 2H-chromene, and benzofuran. Examples of sulfur-containing heterocyclic rings include, but are not limited to, thiophene, benzothiophene, and parathiazine. Examples of nitrogen containing rings include, but are not limited to, pyrrole, pyrrolidine, pyrazole, pyrazolidine, imidazole, imidazoline, imidazolidine, pyridine, piperidine, pyrazine, piperazine, pyrimidine, indole, purine, benzimidazole, quinoline, isoquinoline, triazole, and triazine. Examples of heterocyclic rings containing two different heteroatoms include, but are not limited to, phenothiazine, morpholine, parathiazine, oxazine, oxazole, thiazine, and thiazole. The heterocyclic ring is optionally further substituted at one or more ring positions with, for example, halogen, azide, alkyl, aralkyl, alkenyl, alkynyl, cycloalkyl, hydroxyl, alkoxyl, amino, nitro, sulfhydryl, imino, amido, carboxylic acid, —C(O)alkyl, —CO₂alkyl, carbonyl, carboxyl, alkylthio, sulfonyl, sulfonamido, sulfonamide, ketone, aldehyde, ester, heterocyclyl, aryl or heteroaryl moieties, —CF₃, —CN, or the like.

The terms “amine” and “amino” are art-recognized and refer to both unsubstituted and substituted amines, e.g., a moiety represented by the general formula —N(R⁵⁰)(R⁵¹), wherein R⁵⁰ and R⁵¹ each independently represent hydrogen, alkyl, cycloalkyl, heterocyclyl, alkenyl, aryl, aralkyl, or —(CH₂)ₘ—R⁶¹; or R⁵⁰ and R⁵¹, taken together with the N atom to which they are attached complete a heterocycle having from 4 to 8 atoms in the ring structure; R⁶¹ represents an aryl, a cycloalkyl, a cycloalkenyl, a heterocycle or a polycycle; and m is zero or an integer in the range of 1 to 8. In certain embodiments, R⁵⁰ and R⁵¹ each independently represent hydrogen, alkyl, alkenyl, or —(CH₂)ₘ—R⁶¹.

The terms “alkoxyl” or “alkoxy” are art-recognized and refer to an alkyl group, as defined above, having an oxygen radical attached thereto. Representative alkoxyl groups include methoxy, ethoxy, propyloxy, tert-butoxy and the like. An “ether” is two hydrocarbons covalently linked by an oxygen. Accordingly, the substituent of an alkyl that renders that alkyl an ether is or resembles an alkoxyl, such as may be represented by one of —O-alkyl, —O-alkenyl, —O-alkynyl, —O—(CH₂)ₘ—R₆₁, where m and R₆₁ are described above.

Certain compounds contained in compositions of the present invention may exist in particular geometric or stereoisomeric forms. The present invention contemplates all such compounds, including cis- and trans-isomers, R- and S-enantiomers, diastereomers, (D)-isomers, (L)-isomers, the racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention. It is understood that unless specified otherwise (e.g., using indicators of stereochemical configuration, such as wedge and/or dashed bonds), the chemical formulae encompass all geometric and stereoisomeric forms, including mixtures of geometric and/or stereoisomeric forms.

If, for instance, a particular enantiomer of a compound of the present invention is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic means well known in the art, and subsequent recovery of the pure enantiomers.

As used herein, the terms “subject” and “patient” refer to organisms to be treated by the methods of the present invention. Such organisms are preferably mammals (e.g., murines, simians, equines, bovines, porcines, canines, felines, and the like), and more preferably humans. The term “non-anemic patient” refers to a patient that does not suffer from anemia.

As used herein, the term “effective amount” refers to the amount of a compound (e.g., a compound of the present invention) sufficient to effect beneficial or desired results. An effective amount can be administered in one or more administrations, applications or dosages and is not intended to be limited to a particular formulation or administration route. As used herein, the term “treating” includes any effect, e.g., lessening, reducing, modulating, ameliorating or eliminating, that results in the improvement of the condition, disease, disorder, and the like, or ameliorating a symptom thereof.

As used herein, the term “pharmaceutical composition” refers to the combination of an active agent with a carrier, inert or active, making the composition especially suitable for diagnostic or therapeutic use in vivo or ex vivo.

As used herein, the term “pharmaceutically acceptable carrier” refers to any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants, see e.g., Martin, Remington's Pharmaceutical Sciences, 15^{th} Ed., Mack Publ. Co., Easton, PA [1975].

As used herein, the term “pharmaceutically acceptable salt” refers to any pharmaceutically acceptable salt (e.g., acid or base) of a compound of the present invention which, upon administration to a subject, is capable of providing a compound of this invention or an active metabolite or residue thereof. As is known to those of skill in the art, “salts” of the compounds of the present invention may be derived from inorganic or organic acids and bases. Examples of acids include, but are not limited to, hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, ethanesulfonic, formic, benzoic, malonic, naphthalene-2-sulfonic, benzenesulfonic acid, and the like. Other acids, such as oxalic, while not in themselves pharmaceutically acceptable, may be employed in the preparation of salts useful as intermediates in obtaining the compounds of the invention and their pharmaceutically acceptable acid addition salts.

Examples of bases include, but are not limited to, alkali metals (e.g., sodium) hydroxides, alkaline earth metals (e.g., magnesium), hydroxides, ammonia, and compounds of formula NW₄+, wherein W is C₁₋₄ alkyl, and the like.

Examples of salts include, but are not limited to: acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, flucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, palmoate, pectinate, persulfate, phenylpropionate, picrate, pivalate, propionate, succinate, tartrate, thiocyanate, tosylate, undecanoate, and the like. Other examples of salts include anions of the compounds of the present invention compounded with a suitable cation such as Na⁺, NH₄⁺, and NW₄⁺ (wherein W is a C₁₋₄ alkyl group), and the like.

For therapeutic use, salts of the compounds of the present invention are contemplated as being pharmaceutically acceptable. However, salts of acids and bases that are non-pharmaceutically acceptable may also find use, for example, in the preparation or purification of a pharmaceutically acceptable compound.

The term “RRx-001” refers to 2-bromo-1-(3,3-dinitroazetidin-1-yl)ethanone and is represented by formula (I):

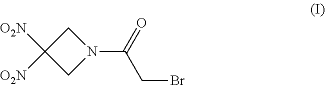 [SMILES: O=C(CBr)N1CC([N+](=O)[O-])([N+](=O)[O-])C1]

The compound may also be referred to as ABDNAZ, and the two terms are used interchangeably throughout the present disclosure.

As used herein, the term “isolated” refers to material that is removed from its original environment (e.g., the natural environment if it is naturally occurring).

Throughout the description, where compositions and kits are described as having, including, or comprising specific components, or where processes and methods are described as having, including, or comprising specific steps, it is contemplated that, additionally, there are compositions and kits of the present invention that consist essentially of, or consist of, the recited components, and that there are processes and methods according to the present invention that consist essentially of, or consist of, the recited processing steps.

As a general matter, compositions specifying a percentage are by weight unless otherwise specified. Further, if a variable is not accompanied by a definition, then the previous definition of the variable controls.

### II. Methods for Treating Disorders

### Treatment Dosing Regimen

Provided herein are methods of treating a disorder selected from the group consisting of an autoimmune disorder, inflammatory disorder, neurodegenerative disorder, and neuromuscular disorder in a subject in need thereof. In certain embodiments, the methods comprise administering a loading dose of RRx-001 or an analog thereof to the subject in an amount effective to ameliorate a symptom of the disorder, and thereafter administering a maintenance dose of RRx-001 or the analog thereof to maintain the amelioration of the symptom for a prolonged period of time. In other embodiments, the RRx-001 or an analog may be administered in an amount and for a time sufficient to ameliorate one or more symptoms of the disorders, where the dosage administered can be the (i) same or similar to a loading dose described herein, (ii) same or similar to a maintenance dose described herein, or (iii) a combination of (i) and (ii) wherein dosage (i) occurs before dosage (ii) or dosage (ii) occurs before dosage (i).

In some embodiments, the analog of RRx-001 is selected from the sulfoxyalkyl organonitro and related compounds as described in section III.

In some embodiments, the loading dose comprises from about 1 mg to about 12 mg, from about 1 mg to about 10 mg, from about 1 mg to about 9 mg, from about 1 mg to about 8 mg, from about 1 mg to about 7 mg, from about 1 mg to about 6 mg, from about 1 mg to about 5 mg, from about 1 mg to about 4 mg, from about 1 mg to about 3 mg, from about 1 mg to about 2 mg, from about 0.1 mg to about 2 mg, from about 0.5 mg to about 5 mg, from about 0.5 mg to about 4.5 mg, from about 0.5 mg to about 4 mg, from about 0.5 mg to about 3.5 mg, from about 0.5 mg to about 3 mg, from about 0.5 mg to about 2.5 mg, from about 0.5 mg to about 2 mg, from about 0.5 mg to about 1.5 mg, or from about 0.5 mg to about 1 mg of RRx-001 or an analog thereof.

In some embodiments, the loading dose comprises about 0.5 mg, about 1 mg, about 1.5 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg, or about 10 mg of RRx-001 or an analog thereof. In certain embodiments, the subject receives a loading dose of at least about 0.5 mg per day, at least about 1 mg per day, or at least about 2 mg per day of RRx-001 or an analog thereof.

In some embodiments, the loading dose comprises from about 1 mg/m² to about 12 mg/m², from about 1 mg/m² to about 10 mg/m², from about 1 mg/m² to about 9 mg/m², from about 1 mg/m² to about 8 mg/m², from about 1 mg/m² to about 7 mg/m², from about 1 mg/m² to about 6 mg/m², from about 1 mg/m² to about 5 mg/m², from about 1 mg/m² to about 4 mg/m², from about 1 mg/m² to about 3 mg/m², from about 1 mg/m² to about 2 mg/m², from about 0.1 mg/m² to about 2 mg/m², from about 0.5 mg/m² to about 5 mg/m², from about 0.5 mg/m² to about 4.5 mg/m², from about 0.5 mg/m² to about 4 mg/m², from about 0.5 mg/m² to about 3.5 mg/m², from about 0.5 mg/m² to about 3 mg/m², from about 0.5 mg/m² to about 2.5 mg/m², from about 0.5 mg/m² to about 2 mg/m², from about 0.5 mg/m² to about 1.5 mg/m², or from about 0.5 mg/m² to about 1 mg/m² of RRx-001 or an analog thereof.

In some embodiments, the loading dose comprises about 0.5 mg/m², about 1 mg/m², about 1.5 mg/m², about 2 mg/m², about 2.5 mg/m², about 3 mg/m², about 3.5 mg/m², about 4 mg/m², about 4.5 mg/m², about 5 mg/m², about 5.5 mg/m², about 6 mg/m², about 6.5 mg/m², about 7 mg/m², about 7.5 mg/m², about 8 mg/m², about 8.5 mg/m², about 9 mg/m², about 9.5 mg/m², or about 10 mg/m² of RRx-001 or an analog thereof. In certain embodiments, the subject receives a loading dose of at least about 0.5 mg/m² per day, at least about 1 mg/m² per day, or at least about 2 mg/m² per day of RRx-001 or an analog thereof.

In some embodiments, the loading dose comprises doses administered after the first day. In some embodiments, the doses administered after the first day are administered with a periodic interval of 1 day to 28 days, 7 days to 28 days, 14 days to 28 days, 21 days to 28 days, 3 days to 14 days, 5 days to 14 days, 7 days to 14 days, 9 days to 14 days, 11 days to 14 days, 13 days to 14 days, 2 days to 7 days, 3 days to 7 days, 4 days to 7 days, 5 days to 7 days, or 6 days to 7 days. In some embodiments, the doses administered after the first day are administered with a periodic interval of 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days 22 days, 23 days, 24 days, 25 days, 26 days, 27 days, or 28 days. In some embodiments, the doses are separated so as to achieve amelioration of the symptom of the disorder.

In some embodiments, the doses administered after the first day comprise from about 1 mg to about 12 mg, from about 1 mg to about 10 mg, from about 1 mg to about 9 mg, from about 1 mg to about 8 mg, from about 1 mg to about 7 mg, from about 1 mg to about 6 mg, from about 1 mg to about 5 mg, from about 1 mg to about 4 mg, from about 1 mg to about 3 mg, from about 1 mg to about 2 mg, from about 0.1 mg to about 2 mg, from about 0.5 mg to about 5 mg, from about 0.5 mg to about 4.5 mg, from about 0.5 mg to about 4 mg, from about 0.5 mg to about 3.5 mg, from about 0.5 mg to about 3 mg, from about 0.5 mg to about 2.5 mg, from about 0.5 mg to about 2 mg, from about 0.5 mg to about 1.5 mg, or from about 0.5 mg to about 1 mg of RRx-001 or an analog thereof.

In some embodiments, the doses administered after the first day comprise about 0.5 mg, about 1 mg, about 1.5 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg, or about 10 mg of RRx-001 or an analog thereof. In certain embodiments, the subject receives doses administered after the first day of at least about 0.5 mg per day, at least about 1 mg per day, or at least about 2 mg per day of RRx-001 or an analog thereof.

In some embodiments, the doses administered after the first day comprise from about 1 mg/m² to about 12 mg/m², from about 1 mg/m² to about 10 mg/m², from about 1 mg/m² to about 9 mg/m², from about 1 mg/m² to about 8 mg/m², from about 1 mg/m² to about 7 mg/m², from about 1 mg/m² to about 6 mg/m², from about 1 mg/m² to about 5 mg/m², from about 1 mg/m² to about 4 mg/m², from about 1 mg/m² to about 3 mg/m², from about 1 mg/m² to about 2 mg/m², from about 0.1 mg/m² to about 2 mg/m², from about 0.5 mg/m² to about 5 mg/m², from about 0.5 mg/m² to about 4.5 mg/m², from about 0.5 mg/m² to about 4 mg/m², from about 0.5 mg/m² to about 3.5 mg/m², from about 0.5 mg/m² to about 3 mg/m², from about 0.5 mg/m² to about 2.5 mg/m², from about 0.5 mg/m² to about 2 mg/m², from about 0.5 mg/m² to about 1.5 mg/m², or from about 0.5 mg/m² to about 1 mg/m² of RRx-001 or an analog thereof.

In some embodiments, the doses administered after the first day comprise about 0.5 mg/m², about 1 mg/m², about 1.5 mg/m², about 2 mg/m², about 2.5 mg/m², about 3 mg/m², about 3.5 mg/m², about 4 mg/m², about 4.5 mg/m², about 5 mg/m², about 5.5 mg/m², about 6 mg/m², about 6.5 mg/m², about 7 mg/m², about 7.5 mg/m², about 8 mg/m², about 8.5 mg/m², about 9 mg/m², about 9.5 mg/m², or about 10 mg/m² of RRx-001 or an analog thereof. In certain embodiments, the subject receives doses administered after the first day of at least about 0.5 mg/m² per day, at least about 1 mg/m² per day, or at least about 2 mg/m² per day of RRx-001 or an analog thereof.

In some embodiments, the doses administered after first day are the same as the dose administered the first day. In some embodiments, the doses administered after the first day are different from the dose administered on the first day. In some embodiments, the doses administered after the first day are larger than the dose administered on the first day. In some embodiments, the doses administered after the first are smaller than the dose administered on the first day.

In some embodiments, the maintenance dose comprises from about 1 mg to about 12 mg, from about 1 mg to about 10 mg, from about 1 mg to about 9 mg, from about 1 mg to about 8 mg, from about 1 mg to about 7 mg, from about 1 mg to about 6 mg, from about 1 mg to about 5 mg, from about 1 mg to about 4 mg, from about 1 mg to about 3 mg, from about 1 mg to about 2 mg, from about 0.1 mg to about 2 mg, from about 0.5 mg to about 5 mg, from about 0.5 mg to about 4.5 mg, from about 0.5 mg to about 4 mg, from about 0.5 mg to about 3.5 mg, from about 0.5 mg to about 3 mg, from about 0.5 mg to about 2.5 mg, from about 0.5 mg to about 2 mg, from about 0.5 mg to about 1.5 mg, or from about 0.5 mg to about 1 mg of RRx-001 or an analog thereof.

In some embodiments, the maintenance dose comprises about 0.5 mg, about 1 mg, about 1.5 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg, or about 10 mg of RRx-001 or an analog thereof. In certain embodiments, the subject receives a maintenance dose of at least at least about 0.5 mg per day, at least about 1 mg per day, or at least about 2 mg per day of RRx-001 or an analog thereof.

In some embodiments, the maintenance dose comprises from about 1 mg/m² to about 12 mg/m², from about 1 mg/m² to about 10 mg/m², from about 1 mg/m² to about 9 mg/m², from about 1 mg/m² to about 8 mg/m², from about 1 mg/m² to about 7 mg/m², from about 1 mg/m² to about 6 mg/m², from about 1 mg/m² to about 5 mg/m², from about 1 mg/m² to about 4 mg/m², from about 1 mg/m² to about 3 mg/m², from about 1 mg/m² to about 2 mg/m², from about 0.1 mg/m² to about 2 mg/m, from about 0.5 mg/m² to about 5 mg/m², from about 0.5 mg/m² to about 4.5 mg/m², from about 0.5 mg/m² to about 4 mg/m², from about 0.5 mg/m² to about 3.5 mg/m², from about 0.5 mg/m² to about 3 mg/m², from about 0.5 mg/m² to about 2.5 mg/m², from about 0.5 mg/m² to about 2 mg/m², from about 0.5 mg/m² to about 1.5 mg/m², or from about 0.5 mg/m² to about 1 mg/m² of RRx-001 or an analog thereof.

In some embodiments, the maintenance dose comprises about 0.5 mg/m², about 1 mg/m², about 1.5 mg/m², about 2 mg/m², about 2.5 mg/m², about 3 mg/m², about 3.5 mg/m², about 4 mg/m², about 4.5 mg/m², about 5 mg/m², about 5.5 mg/m², about 6 mg/m², about 6.5 mg/m², about 7 mg/m², about 7.5 mg/m², about 8 mg/m², about 8.5 mg/m², about 9 mg/m², about 9.5 mg/m², or about 10 mg/m² of RRx-001 or an analog thereof. In certain embodiments, the subject receives a maintenance dose of at least about 0.5 mg/m² per day, at least about 1 mg/m² per day, or at least about 2 mg/m² per day of RRx-001 or an analog thereof.

In some embodiments, the maintenance dose is administered with a periodic interval of 1 day to 28 days, 7 days to 28 days, 14 days to 28 days, 21 days to 28 days, 3 days to 14 days, 5 days to 14 days, 7 days to 14 days, 9 days to 14 days, 11 days to 14 days, 13 days to 14 days, 2 days to 7 days, 3 days to 7 days, 4 days to 7 days, 5 days to 7 days, or 6 days to 7 days. In some embodiments, the maintenance doses are administered with a periodic interval of 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days 22 days, 23 days, 24 days, 25 days, 26 days, 27 days, or 28 days. In some embodiments, the doses are separated so as to maintain amelioration of the symptom of the disorder.

In some embodiments, the maintenance dose is less than the loading dose. In some embodiments, the maintenance dose is the same as the loading dose.

In some embodiments, the prolonged period of time over which amelioration of one or more symptoms is maintained is at least 1 month, 2, 3, 4, 5, 6, 7, 8, 9, 10, 11 or 12 months.

Each dose of RRx-001 or an analog thereof may be administered to the subject by intravenous infusion providing RRx-001 or an analog thereof in any of the amounts described herein. For example, each dose of RRx-001 or an analog thereof may be administered to the subject by intravenous infusion providing RRx-001 or an analog thereof in an amount of from about 0.1 mg to about 10 mg. Each dose of RRx-001 or an analog thereof may be administered to the subject by intravenous infusion providing RRx-001 or an analog thereof in an amount of from about 0.5 mg to about 4.0 mg. Each dose of the formulation comprising RRx-001 or an analog thereof may be administered to the subject by intravenous infusion providing RRx-001 or an analog thereof in an amount of about 0.5 mg, about 1.0 mg, about 1.5 mg, about 2.0 mg, about 2.5 mg, about 3.0 mg, about 3.5 mg, or about 4.0 mg.

Each dose of the formulation comprising RRx-001 or an analog thereof can be administered to the subject by intravenous infusion. Depending on the circumstances, RRx-001 or an analog thereof is pre-mixed with a sample of blood harvested from the subject blood prior to administration. Methods of performing blood transfusion by administering to a subject in need thereof a blood product including RRx-001 is described in WO2017/123593, the content of which is incorporated herein.

In some embodiments, administering the loading dose and/or administering the maintenance dose does not lead to toxic systemic side effects. In some embodiments, the toxic systemic side effects are selected from the group consisting of dyspnea, cough, fatigue, cardiac symptoms, electrolyte deficiencies, thyroid dysfunction, bone loss, sleep issues, nephrotoxicity, neurologic symptoms, autoimmunity, retinitis, hepatotoxicity, gastrointestinal distress, weight loss, malaise, rash, and leukopenia with increased risk of infection and the development of malignancy, and combinations thereof.

### Prophylactic Dosing Regimen

Also provided herein are methods of preventing the initiation, development, or worsening of a symptom of a disorder selected from the group consisting of an autoimmune disorder, inflammatory disorder, neurodegenerative disorder, and neuromuscular disorder to a subject in need thereof. The method comprises administering an effective amount of RRx-001 or an analog thereof to the subject to prevent the initiation, development, or worsening of the symptom of the disorder.

In some embodiments, the effective amount comprises from about 1 mg to about 12 mg, from about 1 mg to about 10 mg, from about 1 mg to about 9 mg, from about 1 mg to about 8 mg, from about 1 mg to about 7 mg, from about 1 mg to about 6 mg, from about 1 mg to about 5 mg, from about 1 mg to about 4 mg, from about 1 mg to about 3 mg, from about 1 mg to about 2 mg, from about 0.1 mg to about 2 mg, from about 0.5 mg to about 5 mg, from about 0.5 mg to about 4.5 mg, from about 0.5 mg to about 4 mg, from about 0.5 mg to about 3.5 mg, from about 0.5 mg to about 3 mg, from about 0.5 mg to about 2.5 mg, from about 0.5 mg to about 2 mg, from about 0.5 mg to about 1.5 mg, or from about 0.5 mg to about 1 mg RRx-001 or an analog thereof.

In some embodiments, the effective amount comprises about 0.5 mg, about 1 mg, about 1.5 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg, or about 10 mg of RRx-001 or an analog thereof. In certain embodiments, the effective amount is at least about 0.5 mg per day, at least about 1 mg per day, or at least about 2 mg per day of RRx-001 or an analog thereof.

In some embodiments, the effective amount comprises from about 1 mg/m² to about 12 mg/m², from about 1 mg/m² to about 10 mg/m², from about 1 mg/m² to about 9 mg/m², from about 1 mg/m² to about 8 mg/m², from about 1 mg/m² to about 7 mg/m², from about 1 mg/m² to about 6 mg/m², from about 1 mg/m² to about 5 mg/m², from about 1 mg/m² to about 4 mg/m², from about 1 mg/m² to about 3 mg/m², from about 1 mg/m² to about 2 mg/m², from about 0.1 mg/m² to about 2 mg/m², from about 0.5 mg/m² to about 5 mg/m², from about 0.5 mg/m² to about 4.5 mg/m², from about 0.5 mg/m² to about 4 mg/m², from about 0.5 mg/m² to about 3.5 mg/m², from about 0.5 mg/m² to about 3 mg/m², from about 0.5 mg/m² to about 2.5 mg/m², from about 0.5 mg/m² to about 2 mg/m², from about 0.5 mg/m² to about 1.5 mg/m², or from about 0.5 mg/m² to about 1 mg/m² of RRx-001 or an analog thereof.

In some embodiments, the effective amount comprises about 0.5 mg/m², about 1 mg/m², about 1.5 mg/m², about 2 mg/m², about 2.5 mg/m², about 3 mg/m², about 3.5 mg/m², about 4 mg/m², about 4.5 mg/m², about 5 mg/m², about 5.5 mg/m², about 6 mg/m², about 6.5 mg/m², about 7 mg/m², about 7.5 mg/m², about 8 mg/m², about 8.5 mg/m², about 9 mg/m², about 9.5 mg/m², or about 10 mg/m² of RRx-001 or an analog thereof. In certain embodiments, the subject receives an effective amount of at least about 0.5 mg/m² per day, at least about 1 mg/m² per day, or at least about 2 mg/m² per day of RRx-001 or an analog thereof.

In some embodiments, the RRx-001 or analog thereof is administered to the subject periodically in an interval of 1 day to 28 days, 7 days to 28 days, 14 days to 28 days, 21 days to 28 days, 3 days to 14 days, 5 days to 14 days, 7 days to 14 days, 9 days to 14 days, 11 days to 14 days, 13 days to 14 days, 2 days to 7 days, 3 days to 7 days, 4 days to 7 days, 5 days to 7 days, or 6 days to 7 days. In some embodiments, the RRx-001 or analog thereof are administered with a periodic interval of 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days 22 days, 23 days, 24 days, 25 days, 26 days, 27 days, or 28 days. In some embodiments, the doses are separated so as to maintain amelioration of the symptom of the disorder.

Each dose of RRx-001 or an analog thereof may be administered prophylactically to the subject by intravenous infusion providing RRx-001 or an analog thereof in any of the amounts described herein. For example, each dose of RRx-001 or an analog thereof may be administered to the subject by intravenous infusion providing RRx-001 or an analog thereof in an amount of from about 0.1 mg to about 10 mg. Each dose of RRx-001 or an analog thereof may be administered to the subject by intravenous infusion providing RRx-001 or an analog thereof in an amount of from about 0.5 mg to about 4.0 mg. Each dose of the formulation comprising RRx-001 or an analog thereof may be administered to the subject by intravenous infusion providing RRx-001 or an analog thereof in an amount of about 0.5 mg, about 1.0 mg, about 1.5 mg, about 2.0 mg, about 2.5 mg, about 3.0 mg, about 3.5 mg, or about 4.0 mg.

Each dose of the formulation comprising RRx-001 or an analog thereof can be administered to the subject by intravenous infusion. Depending on the circumstances, RRx-001 or an analog thereof is pre-mixed with a sample of blood harvested from the subject blood prior to administration. Methods of performing blood transfusion by administering to a subject in need thereof a blood product including RRx-001 is described in WO2017/123593, the content of which is incorporated herein.

In some embodiments, administering the effective amount of RRx-001 or an analog thereof does not lead to toxic systemic side effects. In some embodiments, the toxic systemic side effects are selected from the group consisting of dyspnea, cough, fatigue, cardiac symptoms, electrolyte deficiencies, thyroid dysfunction, bone loss, sleep issues, nephrotoxicity, neurologic symptoms, autoimmunity, retinitis, hepatotoxicity, gastrointestinal distress, weight loss, malaise, rash, and leukopenia with increased risk of infection and the development of malignancy, and combinations thereof.

### III. Organonitro, Sulfoxyalkyl Organonitro, and Related Compounds

One aspect of the invention provides organonitro or sulfoxyalkyl organonitro and related compounds. The compounds are useful in the methods, compositions, and kits described herein. In certain embodiments, the organonitro compound is RRx-001, represented by formula (I):

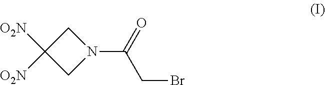 [SMILES: O=C(CBr)N1CC([N+](=O)[O-])([N+](=O)[O-])C1]

See, for example, U.S. Pat. No. 10,149,832 for the synthesis of the compound of formula I as well as other organonitro compounds useful in the practice of the invention.

In some embodiments, the analog of RRx-001 is selected from the sulfoxyalkyl organonitro and related compounds as described below (e.g., a compound of formula II, III).

In certain embodiments, the sulfoxyalkyl organonitro compound is a compound embraced by Formula II:

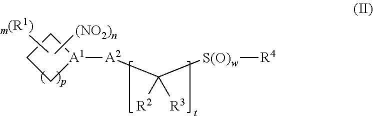

or a pharmaceutically acceptable salt or solvate thereof, wherein:
A¹ is N or —C(R⁵)—;
A² is —C(O)— or —(C(R⁶)₂)ₓC(O)(C(R⁶)₂)ₓ—;
R¹ is C₁-C₅alkyl;
R² and R³ each represent independently for each occurrence hydrogen or C₁-C₅alkyl; or R² and R³ are taken together with the carbon atom to which they are attached to form a carbocyclic ring;
R⁴ is C₁-C₅alkyl substituted with one X¹ group and one X² group; wherein X¹ is —N(R⁷)(R⁸), —N(R⁷)C(O)ₖ—C₁-C₅alkyl, —N(R⁷)C(O)ₖ—C₃-C₇cycloalkyl, —N(R⁷)C(O)ₖ-aryl, —N(R⁷)C(O)ₖ-aralkyl, or —N(R⁷)C(O)—(C₁-C₅alkylene)-C(H)[N(R⁷)(R⁸)]—CO₂R⁹; and X² is —CO₂R¹⁰ or —C(O)N(R⁷)—(C₁-C₅alkylene)-CO₂R¹⁰;
R⁵ is hydrogen or C₁-C₅alkyl;
R⁶ represents independently for each occurrence C₁-C₆alkyl, C₁-C₅haloalkyl, aryl, or aralkyl;
R⁷ and R⁸ each represent independently for each occurrence hydrogen or C₁-C₅alkyl; or R⁷ and R⁸ are taken together with the nitrogen atom to which they are attached to form a 3-7 membered heterocyclic ring;
R⁹ and R¹⁰ each represent independently hydrogen, C₁-C₅alkyl, C₃-C₇cycloalkyl, aryl, or aralkyl;
k and w are independently 1 or 2;
n, p, and t are independently 1, 2, or 3; and
m and x each represent independently for each occurrence 0, 1, 2, 3, or 4.

In certain embodiments, A¹ is N. In certain embodiments, A² is —C(O)—.

In certain embodiments, R² and R³ are hydrogen.

In certain embodiments, m is 0. In certain embodiments, n is 2. In certain other embodiments, n is 1. In certain embodiments, t is 1. In certain embodiments, p is 1.

In certain embodiments, k is 1. In certain embodiments, k is 2.

In certain embodiments, R⁴ is —CH₂C(H)(X¹)X². In certain other embodiments, R⁴ is

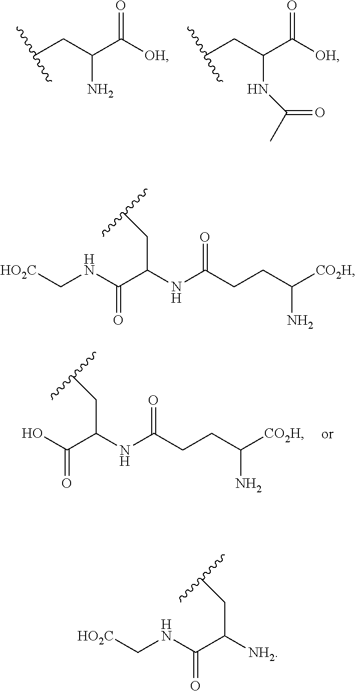

In certain other embodiments, R⁴ is

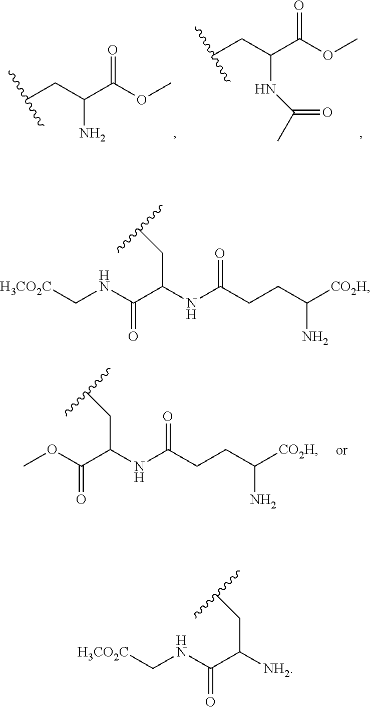

In certain embodiments, X¹ is —N(R⁷)(R⁸), —N(R⁷)C(O)—C₁-C₅alkyl, or —N(R⁷)C(O)—(C₁-C₅alkylene)-C(H)[N(R⁷)(R⁸)]—CO₂R⁹. In certain other embodiments, X¹ is —NH₂, —N(H)C(O)CH₂, or —N(H)C(O)CH₂CH₂C(H)(NH₂)—CO₂H; and X² is —CO₂H, —CO₂Me, or —C(O)N(H)CH₂CO₂H. In certain other embodiments, X¹ is —NH₂ or —N(H)C(O)CH₂CH₂C(H)(NH₂)—CO₂H; and X² is —CO₂H or —C(O)N(H)CH₂CO₂H.

The description above describes multiple embodiments relating to compounds of Formula II. The patent application specifically contemplates all combinations of the embodiments. For example, the invention contemplates a compound of Formula II wherein A¹ is N, A² is —C(O)—, R² and R³ are hydrogen, m is 0, n is 2, t is 1, and R⁴ is —CH₂C(H)(X¹)X². Further, to illustrate, the invention contemplates a compound of Formula II wherein A¹ is N, A² is —C(O)—, R² and R³ are hydrogen, m is 0, n is 1, t is 1, and R⁴ is —CH₂C(H)(X¹)X².

In certain embodiments, the compound is a compound of Formula II-A:

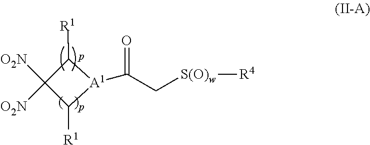

or a pharmaceutically acceptable salt or solvate thereof, wherein:
A¹ is N or C(H);
R¹ represents independently for each occurrence hydrogen or methyl;
R⁴ is C₁-C₅alkyl substituted with one X¹ group and one X² group; wherein X¹ is —NH₂, —N(H)C(O)—C₁-C₅alkyl, or —N(H)C(O)—(C₁-C₅alkylene)-C(H)(NH₂)—CO₂H; and X² is —CO₂H, —CO₂—C₁-C₅alkyl, or —C(O)N(H)CH₂CO₂H;
p represents independently for each occurrence 1 or 2; and
w is 1 or 2.

In certain embodiments, R⁴ is —CH₂C(H)(X¹)X². In certain other embodiments, wherein R⁴ is

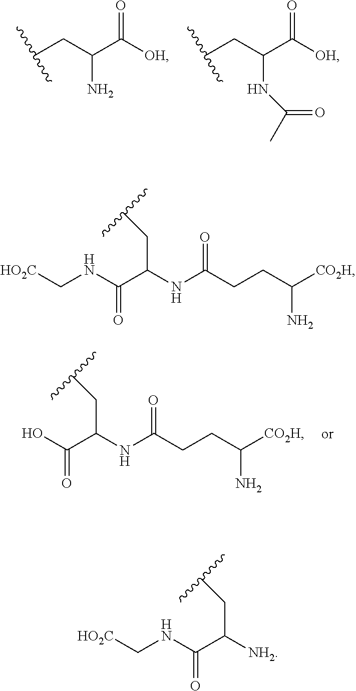

In certain embodiments, p is 1. In certain embodiments, w is 1. In certain embodiments, w is 2.

In certain embodiments, X¹ is —NH₂, —N(H)C(O)CH₃, or —N(H)C(O)CH₂CH₂C(H)(NH₂)—CO₂H; and X² is —CO₂H, —CO₂Me, or —C(O)N(H)CH₂CO₂H. In certain embodiments. X¹ is —NH₂ or —N(H)C(O)CH₂CH₂C(H)(NH₂)—CO₂H; and X² is —CO₂H or —C(O)N(H)CH₂CO₂H.

In certain embodiments, the organonitro compound is represented by

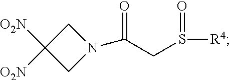

wherein R⁴ is

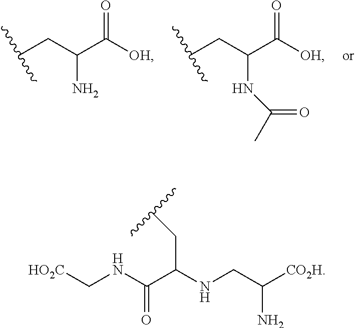

The description above describes multiple embodiments relating to compounds of Formula II-A. The patent application specifically contemplates all combinations of the embodiments. For example, the invention contemplates a compound of Formula II-A wherein A¹ is N, R¹ is hydrogen, R⁴ is —CH₂C(H)(X¹)X², and p is 1.

In certain embodiments, the compound is a compound of Formula II-B:

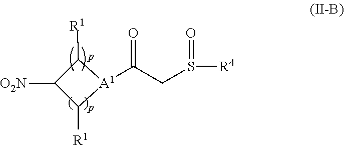

or a pharmaceutically acceptable salt or solvate thereof, wherein:
A¹ is N or C(H);
R¹ represents independently for each occurrence hydrogen or methyl;
R⁴ is C₁-C₅alkyl substituted with one X¹ group and one X² group; wherein X¹ is —NH₂, —N(H)C(O)—C₁-C₅alkyl, or —N(H)C(O)—C₁-C₅alkylene)-C(H)(NH₂)—CO₂H; and X² is —CO₂H, —CO₂—C₁-C₅alkyl, or —C(O)N(H)CH₂CO₂H; and
p represents independently for each occurrence 1 or 2.

In certain embodiments, R⁴ is —CH₂C(H)(X¹)X². In certain other embodiments, wherein R⁴ is

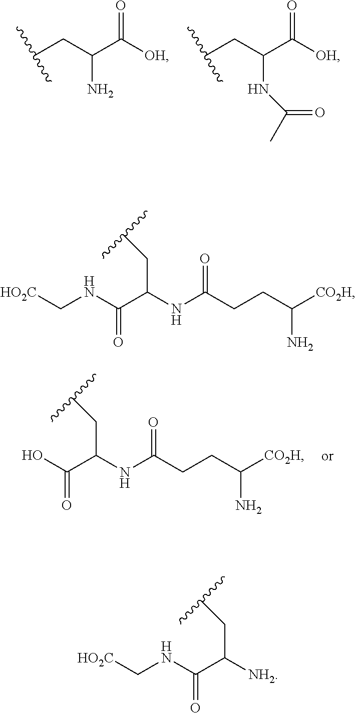

The description above describes multiple embodiments relating to compounds of Formula II-B. The patent application specifically contemplates all combinations of the embodiments. For example, the invention contemplates a compound of Formula II-B wherein A¹ is N, R¹ is hydrogen, R⁴ is —CH₂C(H)(X¹)X², and p is 1.

In certain embodiments, the compound is one of the following:

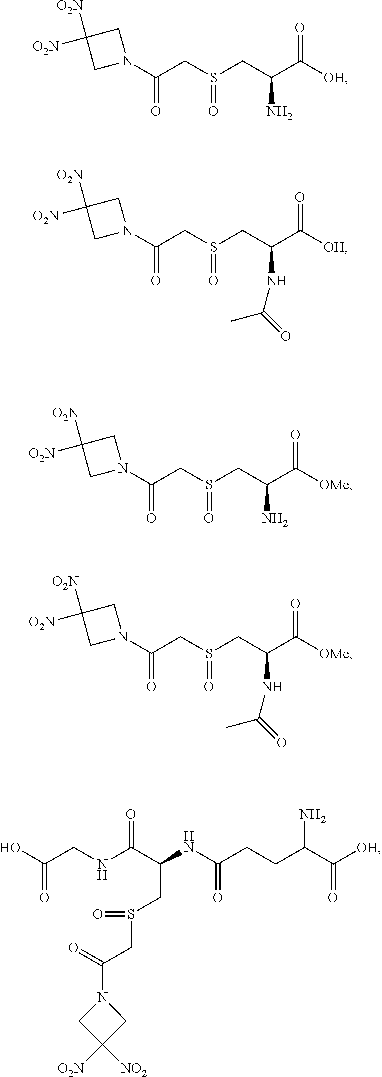 [SMILES: N[C@@H](CS(=O)CC(=O)N1CC([N+](=O)[O-])([N+](=O)[O-])C1)C(=O)O ; CC(=O)N[C@@H](CS(=O)CC(=O)N1CC([N+](=O)[O-])([N+](=O)[O-])C1)C(=O)O ; COC(=O)[C@@H](N)CS(=O)CC(=O)N1CC([N+](=O)[O-])([N+](=O)[O-])C1 ; COC(=O)[C@H](CS(=O)CC(=O)N1CC([N+](=O)[O-])([N+](=O)[O-])C1)NC(C)=O ; NC(CCC(=O)N[C@@H](CS(=O)CC(=O)N1CC([N+](=O)[O-])([N+](=O)[O-])C1)C(=O)NCC(=O)O)C(=O)O]

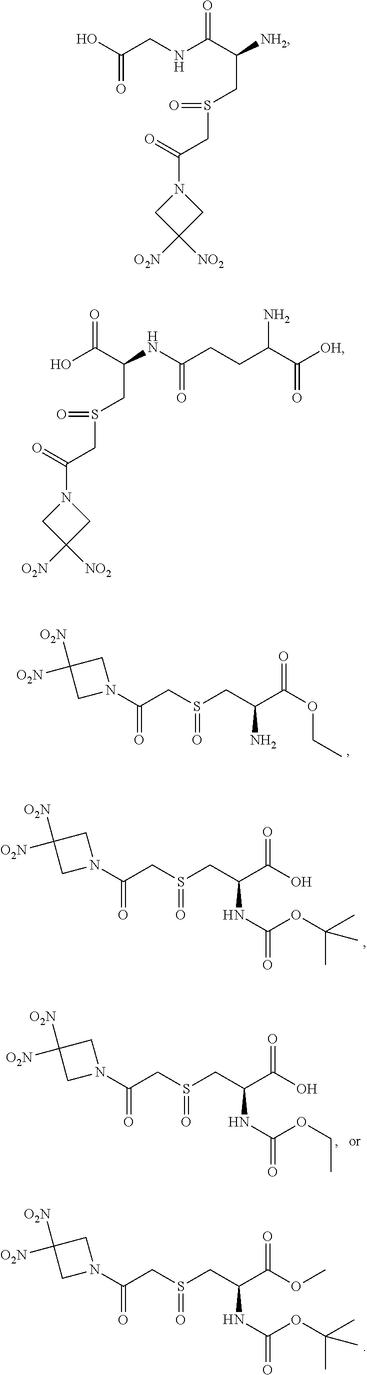 [SMILES: N[C@@H](CS(=O)CC(=O)N1CC([N+](=O)[O-])([N+](=O)[O-])C1)C(=O)NCC(=O)O ; NC(CCC(=O)N[C@@H](CS(=O)CC(=O)N1CC([N+](=O)[O-])([N+](=O)[O-])C1)C(=O)O)C(=O)O ; CCOC(=O)[C@@H](N)CS(=O)CC(=O)N1CC([N+](=O)[O-])([N+](=O)[O-])C1 ; CC(C)(C)OC(=O)N[C@@H](CS(=O)CC(=O)N1CC([N+](=O)[O-])([N+](=O)[O-])C1)C(=O)O ; CCOC(=O)N[C@@H](CS(=O)CC(=O)N1CC([N+](=O)[O-])([N+](=O)[O-])C1)C(=O)O ; COC(=O)[C@H](CS(=O)CC(=O)N1CC([N+](=O)[O-])([N+](=O)[O-])C1)NC(=O)OC(C)(C)C]

In certain embodiments, the compound is one of the foregoing or a pharmaceutically acceptable salt thereof, for example, a hydrochloride salt thereof.

In certain embodiments, the compound is one of the following:

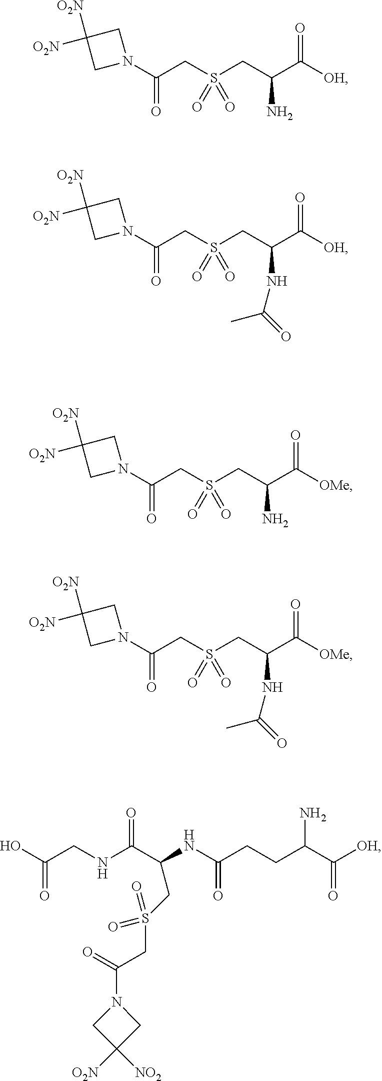 [SMILES: N[C@@H](CS(=O)(=O)CC(=O)N1CC([N+](=O)[O-])([N+](=O)[O-])C1)C(=O)O ; CC(=O)N[C@@H](CS(=O)(=O)CC(=O)N1CC([N+](=O)[O-])([N+](=O)[O-])C1)C(=O)O ; COC(=O)[C@@H](N)CS(=O)(=O)CC(=O)N1CC([N+](=O)[O-])([N+](=O)[O-])C1 ; COC(=O)[C@H](CS(=O)(=O)CC(=O)N1CC([N+](=O)[O-])([N+](=O)[O-])C1)NC(C)=O ; NC(CCC(=O)N[C@@H](CS(=O)(=O)CC(=O)N1CC([N+](=O)[O-])([N+](=O)[O-])C1)C(=O)NCC(=O)O)C(=O)O]

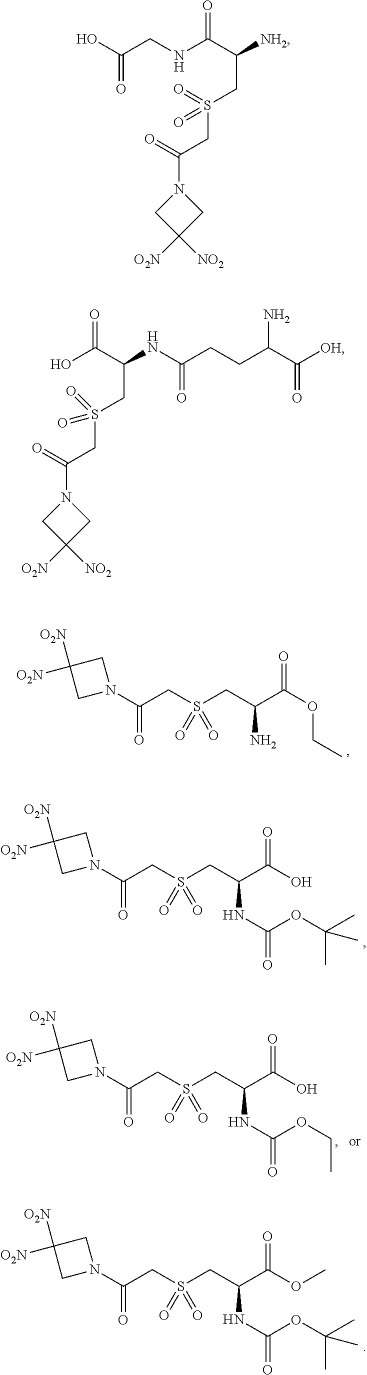 [SMILES: N[C@@H](CS(=O)(=O)CC(=O)N1CC([N+](=O)[O-])([N+](=O)[O-])C1)C(=O)NCC(=O)O ; NC(CCC(=O)N[C@@H](CS(=O)(=O)CC(=O)N1CC([N+](=O)[O-])([N+](=O)[O-])C1)C(=O)O)C(=O)O ; CCOC(=O)[C@@H](N)CS(=O)(=O)CC(=O)N1CC([N+](=O)[O-])([N+](=O)[O-])C1 ; CC(C)(C)OC(=O)N[C@@H](CS(=O)(=O)CC(=O)N1CC([N+](=O)[O-])([N+](=O)[O-])C1)C(=O)O ; CCOC(=O)N[C@@H](CS(=O)(=O)CC(=O)N1CC([N+](=O)[O-])([N+](=O)[O-])C1)C(=O)O ; COC(=O)[C@H](CS(=O)(=O)CC(=O)N1CC([N+](=O)[O-])([N+](=O)[O-])C1)NC(=O)OC(C)(C)C]

In certain embodiments, the compound is one of the foregoing or a pharmaceutically acceptable salt thereof.

In certain other embodiments, the sulfoxyalkyl organonitro compound is a compound embraced by Formula III:

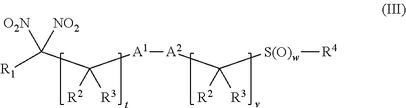

or a pharmaceutically acceptable salt or solvate thereof: wherein:
A¹ is —N(R⁵)—or —C(R²)R³)—;
A² is —C(O)— or —(C(R⁶)₂)ₓC(O)(C(R⁶)₂)ₓ—;
R¹ is C₁-C₅alkyl or C₃-C₇cycloalkyl;
R² and R³ each represent independently for each occurrence hydrogen or C₁-C₅alkyl; or R² and R³ are taken together with the carbon atom to which they are attached to form a carbocyclic ring;
R⁴ is C₁-C₅alkyl substituted with one X¹ group and one X² group; wherein X¹ is —N(R⁷)(R⁸), —N(R⁷)C(O)ₖ—C₁-C₅alkyl, —N(R⁷)C(O)ₖ—C₃-C₇cycloalkyl, —N(R⁷)C(O)ₖ-aryl, —N(R⁷)C(O)ₖ-aralkyl, or —N(R⁷)C(O)—(C₁-C₅alkylene)-C(H)[N(R⁷)(R⁸)]—CO₂R⁹; and X² is —CO₂R¹⁰ or —C(O)N(R⁷)—(C₁-C₅alkylene)-CO₂R¹⁰;
R⁵ is hydrogen or C₁-C₅alkyl;
R⁶ represents independently for each occurrence C₁-C₅alkyl, C₁-C₅haloalkyl, aryl, or aralkyl;
R⁷ and R⁸ each represent independently for each occurrence hydrogen or C₁-C₅alkyl; or R⁷ and R⁸ are taken together with the nitrogen atom to which they are attached to form a 3-7 membered heterocyclic ring;
R⁹ and R¹⁰ each represent independently hydrogen, C₁-C₅alkyl, C₃-C₇cycloalkyl, aryl, or aralkyl;
k and w are independently 1 or 2;
t and v are independently 1, 2, or 3; and
x represents independently for each occurrence 0, 1, 2, 3, or 4.

In certain embodiments, A¹ is N. In certain embodiments, A² is —C(O)—.

In certain embodiments, R² and R³ are hydrogen.

In certain embodiments, m is 0. In certain embodiments, n is 2. In certain other embodiments, n is 1. In certain embodiments, t is 1. In certain embodiments, v is 1.

In certain embodiments, k is 1. In certain embodiments, k is 2.

In certain embodiments, R⁴ is —CH₂C(H)(X¹)X². In certain other embodiments, R⁴ is

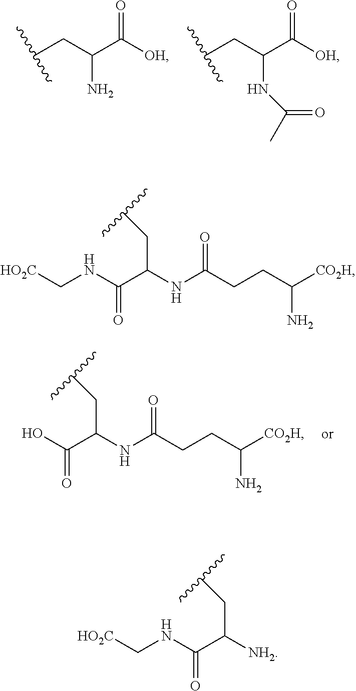

In certain other embodiments, R⁴ is

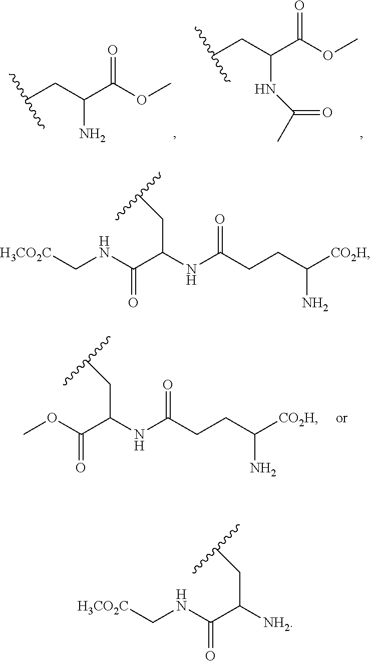

In certain embodiments, X¹ is —N(R⁷)(R⁸), —N(R⁷)C(O)—C₁-C₅alkyl, or —N(R⁷)C(O)—(C₁-C₅alkylene)-C(H)[N(R⁷)(R⁸)]—CO₂R⁹. In certain other embodiments, X¹ is —NH₂, —N(H)C(O)CH₂, or —N(H)C(O)CH₂CH₂C(H)(NH₂)—CO₂H; and X² is —CO₂H, —CO₂Me, or —C(O)N(H)CH₂CO₂H. In certain other embodiments, X¹ is —NH₂

or —N(H)C(O)CH₂CH₂C(H)(NH₂)—CO₂H; and X² is —CO₂H or —C(O)N(H)CH₂CO₂H.

The description above describes multiple embodiments relating to compounds of Formula III. The patent application specifically contemplates all combinations of the embodiments. For example, the invention contemplates a compound of Formula III wherein A¹ is N, A² is —C(O)—, R² and R³ are hydrogen, t is 1, v is 1, and R⁴ is —CH₂C(H)(X¹)X².

In certain embodiments, the invention provides an organonitro or sulfoxyalkyl organonitro or related compound (e.g., a compound of Formula I, II or III) in isolated form. For example, in certain embodiments, the invention provides compounds of Formula II in isolated form. In another embodiment, the isolated compound of Formula II is substantially pure (that is having a purity of at least about 70%, 80%, 90%, 95%, or 99% by weight). In certain embodiments, the invention provides compounds of Formula II-A in isolated form. In another embodiment, the isolated compound of Formula II-A is substantially pure (that is having a purity of at least about 70%, 80%, 90%, 95%, or 99% by weight).

In yet other embodiments, the isolated compound may be one of the following isolated compounds:

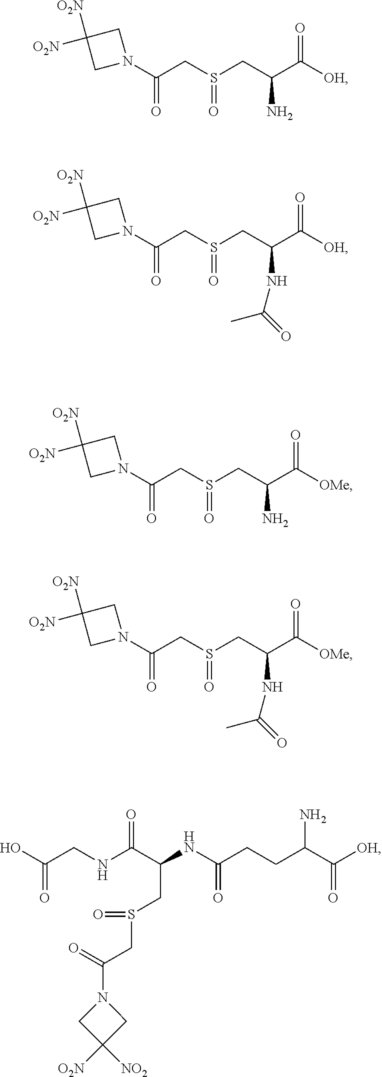 [SMILES: N[C@@H](CS(=O)CC(=O)N1CC([N+](=O)[O-])([N+](=O)[O-])C1)C(=O)O ; CC(=O)N[C@@H](CS(=O)CC(=O)N1CC([N+](=O)[O-])([N+](=O)[O-])C1)C(=O)O ; COC(=O)[C@@H](N)CS(=O)CC(=O)N1CC([N+](=O)[O-])([N+](=O)[O-])C1 ; COC(=O)[C@H](CS(=O)CC(=O)N1CC([N+](=O)[O-])([N+](=O)[O-])C1)NC(C)=O ; NC(CCC(=O)N[C@@H](CS(=O)CC(=O)N1CC([N+](=O)[O-])([N+](=O)[O-])C1)C(=O)NCC(=O)O)C(=O)O]

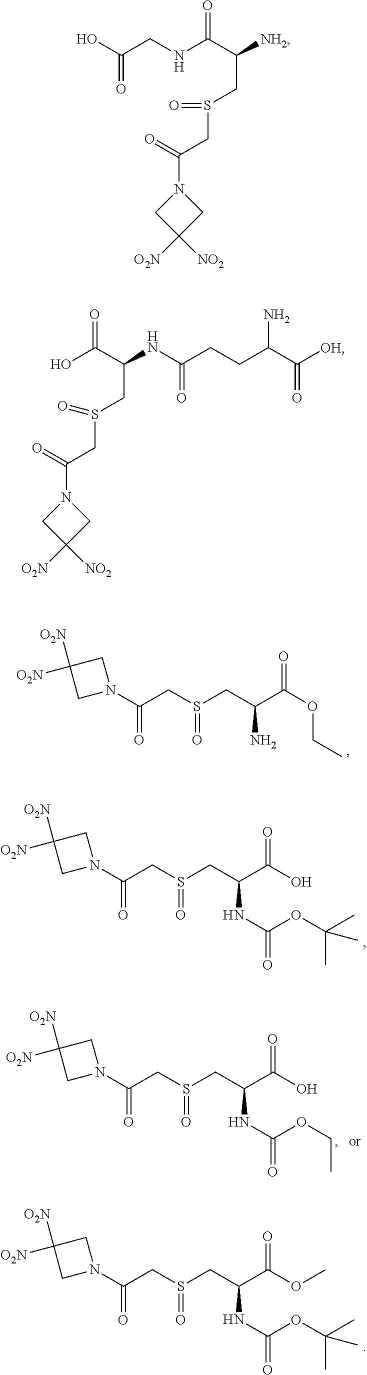 [SMILES: N[C@@H](CS(=O)CC(=O)N1CC([N+](=O)[O-])([N+](=O)[O-])C1)C(=O)NCC(=O)O ; NC(CCC(=O)N[C@@H](CS(=O)CC(=O)N1CC([N+](=O)[O-])([N+](=O)[O-])C1)C(=O)O)C(=O)O ; CCOC(=O)[C@@H](N)CS(=O)CC(=O)N1CC([N+](=O)[O-])([N+](=O)[O-])C1 ; CC(C)(C)OC(=O)N[C@@H](CS(=O)CC(=O)N1CC([N+](=O)[O-])([N+](=O)[O-])C1)C(=O)O ; CCOC(=O)N[C@@H](CS(=O)CC(=O)N1CC([N+](=O)[O-])([N+](=O)[O-])C1)C(=O)O ; COC(=O)[C@H](CS(=O)CC(=O)N1CC([N+](=O)[O-])([N+](=O)[O-])C1)NC(=O)OC(C)(C)C]

In certain embodiments, the isolated compound is one of the foregoing or a pharmaceutically acceptable salt thereof.

In certain other embodiments, the invention provides compounds of Formula III in isolated form. In another embodiment, the isolated compound of Formula II is substantially pure (that is having a purity of at least about 70%, 80%, 90%, 95%, or 99% by weight).

In certain other embodiments, the compound is one of the compounds listed in Tables 1, 2, or 3 below or a pharmaceutically acceptable salt or solvate thereof.

TABLE 1

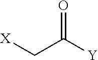

| Compound | | |
|---|---|---|
| No. | X | Y |
| I-1 | —Br | 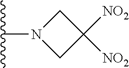 [SMILES: *N1CC([N+](=O)[O-])([N+](=O)[O-])C1] |
| II-1 | 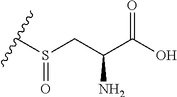 [SMILES: *S(=O)C[C@H](N)C(=O)O] | 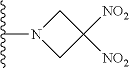 [SMILES: *N1CC([N+](=O)[O-])([N+](=O)[O-])C1] |
| II-2 | 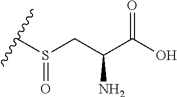 [SMILES: *S(=O)C[C@H](N)C(=O)O] | 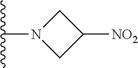 [SMILES: *N1CC([N+](=O)[O-])C1] |
| II-3 | 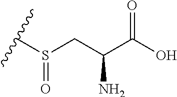 [SMILES: *S(=O)C[C@H](N)C(=O)O] | 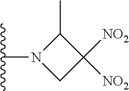 [SMILES: *N1CC([N+](=O)[O-])([N+](=O)[O-])C1C] |
| II-4 | 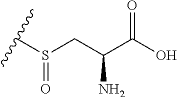 [SMILES: *S(=O)C[C@H](N)C(=O)O] | 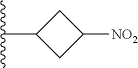 [SMILES: *C1CC([N+](=O)[O-])C1] |
| II-5 | 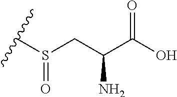 [SMILES: *S(=O)C[C@H](N)C(=O)O] | 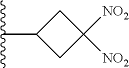 [SMILES: *C1CC([N+](=O)[O-])([N+](=O)[O-])C1] |
| II-6 | 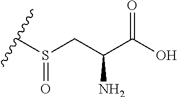 [SMILES: *S(=O)C[C@H](N)C(=O)O] | 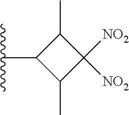 [SMILES: *C1C(C)C([N+](=O)[O-])([N+](=O)[O-])C1C] |
| II-7 | 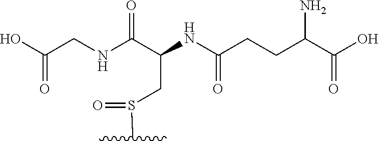 [SMILES: *S(=O)C[C@H](NC(=O)CCC(N)C(=O)O)C(=O)NCC(=O)O] | 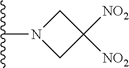 [SMILES: *N1CC([N+](=O)[O-])([N+](=O)[O-])C1] |
| II-8 | 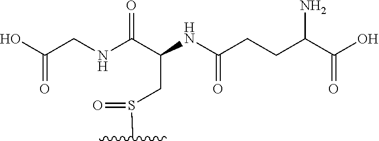 [SMILES: *S(=O)C[C@H](NC(=O)CCC(N)C(=O)O)C(=O)NCC(=O)O] | 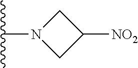 [SMILES: *N1CC([N+](=O)[O-])C1] |
| II-9 | 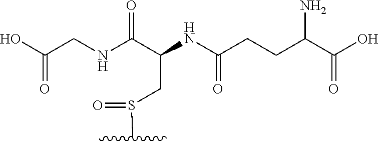 [SMILES: *S(=O)C[C@H](NC(=O)CCC(N)C(=O)O)C(=O)NCC(=O)O] | 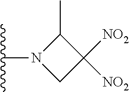 [SMILES: *N1CC([N+](=O)[O-])([N+](=O)[O-])C1C] |
| II-10 | 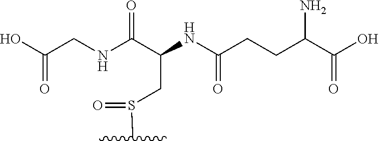 [SMILES: *S(=O)C[C@H](NC(=O)CCC(N)C(=O)O)C(=O)NCC(=O)O] | 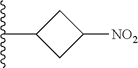 [SMILES: *C1CC([N+](=O)[O-])C1] |
| II-11 | 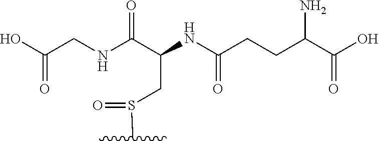 [SMILES: *S(=O)C[C@H](NC(=O)CCC(N)C(=O)O)C(=O)NCC(=O)O] | 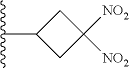 [SMILES: *C1CC([N+](=O)[O-])([N+](=O)[O-])C1] |
| II-12 | 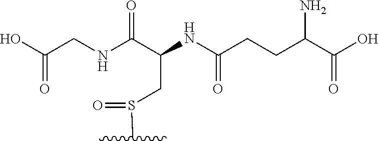 [SMILES: *S(=O)C[C@H](NC(=O)CCC(N)C(=O)O)C(=O)NCC(=O)O] | 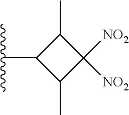 [SMILES: *C1C(C)C([N+](=O)[O-])([N+](=O)[O-])C1C] |
| II-13 | 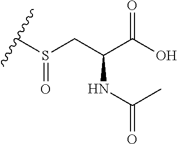 [SMILES: *S(=O)C[C@H](NC(C)=O)C(=O)O] | 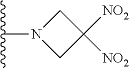 [SMILES: *N1CC([N+](=O)[O-])([N+](=O)[O-])C1] |
| II-14 | 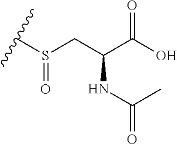 [SMILES: *S(=O)C[C@H](NC(C)=O)C(=O)O] | 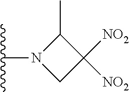 [SMILES: *N1CC([N+](=O)[O-])([N+](=O)[O-])C1C] |
| II-15 | 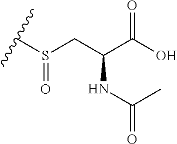 [SMILES: *S(=O)C[C@H](NC(C)=O)C(=O)O] | 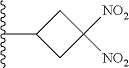 [SMILES: *C1CC([N+](=O)[O-])([N+](=O)[O-])C1] |
| II-16 | 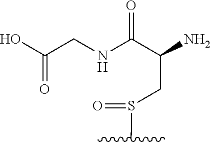 [SMILES: *S(=O)C[C@H](N)C(=O)NCC(=O)O] | 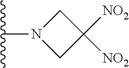 [SMILES: *N1CC([N+](=O)[O-])([N+](=O)[O-])C1] |
| II-17 | 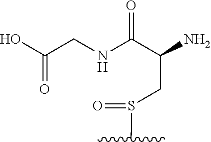 [SMILES: *S(=O)C[C@H](N)C(=O)NCC(=O)O] | 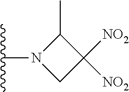 [SMILES: *N1CC([N+](=O)[O-])([N+](=O)[O-])C1C] |
| II-18 | 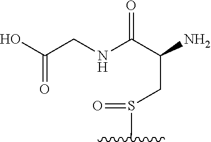 [SMILES: *S(=O)C[C@H](N)C(=O)NCC(=O)O] | 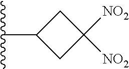 [SMILES: *C1CC([N+](=O)[O-])([N+](=O)[O-])C1] |
| II-19 | 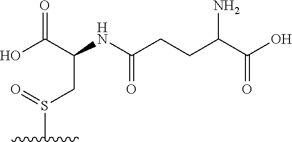 [SMILES: *S(=O)C[C@H](NC(=O)CCC(N)C(=O)O)C(=O)O] | 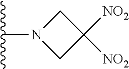 [SMILES: *N1CC([N+](=O)[O-])([N+](=O)[O-])C1] |
| II-20 | 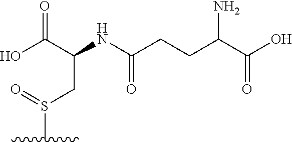 [SMILES: *S(=O)C[C@H](NC(=O)CCC(N)C(=O)O)C(=O)O] | 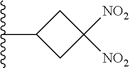 [SMILES: *C1CC([N+](=O)[O-])([N+](=O)[O-])C1] |
| II-21 | 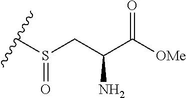 [SMILES: *S(=O)C[C@H](N)C(=O)OC] | 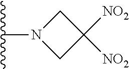 [SMILES: *N1CC([N+](=O)[O-])([N+](=O)[O-])C1] |
| II-22 | 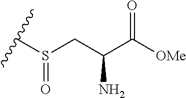 [SMILES: *S(=O)C[C@H](N)C(=O)OC] | 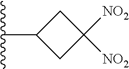 [SMILES: *C1CC([N+](=O)[O-])([N+](=O)[O-])C1] |
| II-23 | 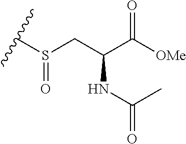 [SMILES: *S(=O)C[C@H](NC(C)=O)C(=O)OC] | 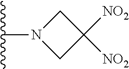 [SMILES: *N1CC([N+](=O)[O-])([N+](=O)[O-])C1] |
| II-24 | 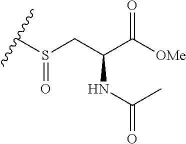 [SMILES: *S(=O)C[C@H](NC(C)=O)C(=O)OC] | 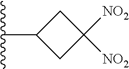 [SMILES: *C1CC([N+](=O)[O-])([N+](=O)[O-])C1] |
| II-25 | 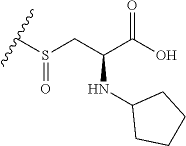 [SMILES: *S(=O)C[C@H](NC1CCCC1)C(=O)O] | 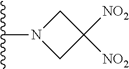 [SMILES: *N1CC([N+](=O)[O-])([N+](=O)[O-])C1] |
| II-26 | 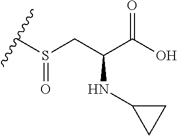 [SMILES: *S(=O)C[C@H](NC1CC1)C(=O)O] | 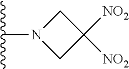 [SMILES: *N1CC([N+](=O)[O-])([N+](=O)[O-])C1] |
| II-27 | 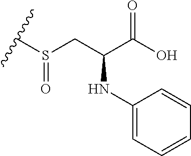 [SMILES: *S(=O)C[C@H](Nc1ccccc1)C(=O)O] | 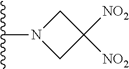 [SMILES: *N1CC([N+](=O)[O-])([N+](=O)[O-])C1] |
| II-28 | 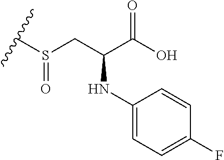 [SMILES: *S(=O)C[C@H](Nc1ccc(F)cc1)C(=O)O] | 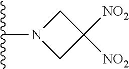 [SMILES: *N1CC([N+](=O)[O-])([N+](=O)[O-])C1] |
| II-29 | 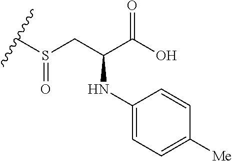 [SMILES: *S(=O)C[C@H](Nc1ccc(C)cc1)C(=O)O] | 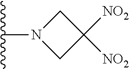 [SMILES: *N1CC([N+](=O)[O-])([N+](=O)[O-])C1] |
| II-30 | 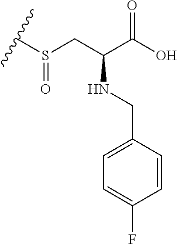 [SMILES: *S(=O)C[C@H](NCc1ccc(F)cc1)C(=O)O] | 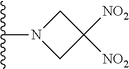 [SMILES: *N1CC([N+](=O)[O-])([N+](=O)[O-])C1] |
| II-31 | 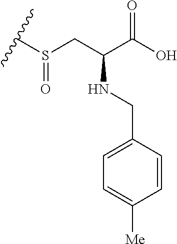 [SMILES: *S(=O)C[C@H](NCc1ccc(C)cc1)C(=O)O] | 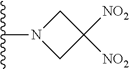 [SMILES: *N1CC([N+](=O)[O-])([N+](=O)[O-])C1] |
| II-32 | 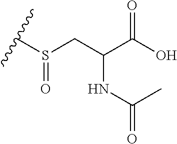 [SMILES: *S(=O)CC(NC(C)=O)C(=O)O] | 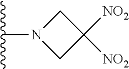 [SMILES: *N1CC([N+](=O)[O-])([N+](=O)[O-])C1] |
| II-33 | 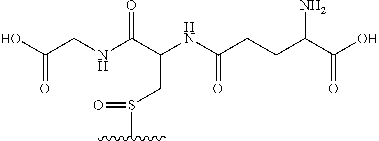 [SMILES: *S(=O)CC(NC(=O)CCC(N)C(=O)O)C(=O)NCC(=O)O] | 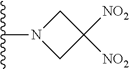 [SMILES: *N1CC([N+](=O)[O-])([N+](=O)[O-])C1] |
| II-34 | 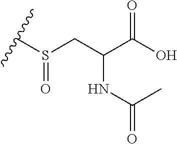 [SMILES: *S(=O)CC(NC(C)=O)C(=O)O] | 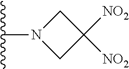 [SMILES: *N1CC([N+](=O)[O-])([N+](=O)[O-])C1] |
| II-35 | 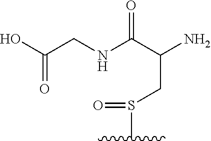 [SMILES: *S(=O)CC(N)C(=O)NCC(=O)O] | 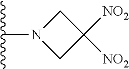 [SMILES: *N1CC([N+](=O)[O-])([N+](=O)[O-])C1] |
| II-36 | 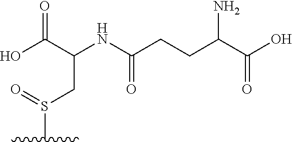 [SMILES: *S(=O)CC(NC(=O)CCC(N)C(=O)O)C(=O)O] | 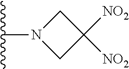 [SMILES: *N1CC([N+](=O)[O-])([N+](=O)[O-])C1] |
| II-37 | 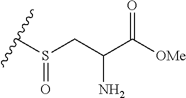 [SMILES: *S(=O)CC(N)C(=O)OC] | 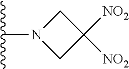 [SMILES: *N1CC([N+](=O)[O-])([N+](=O)[O-])C1] |
| II-38 | 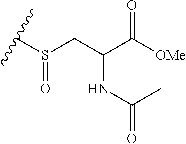 [SMILES: *S(=O)CC(NC(C)=O)C(=O)OC] | 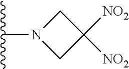 [SMILES: *N1CC([N+](=O)[O-])([N+](=O)[O-])C1] |
| II-39 | 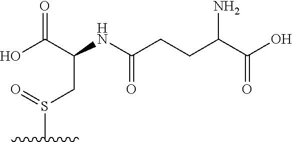 [SMILES: *S(=O)C[C@H](NC(=O)CCC(N)C(=O)O)C(=O)O] | 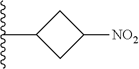 [SMILES: *C1CC([N+](=O)[O-])C1] |
| II-40 | 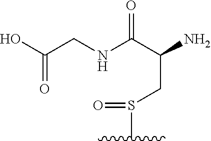 [SMILES: *S(=O)C[C@H](N)C(=O)NCC(=O)O] | 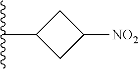 [SMILES: *C1CC([N+](=O)[O-])C1] |
| II-41 | 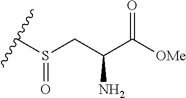 [SMILES: *S(=O)C[C@H](N)C(=O)OC] | 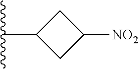 [SMILES: *C1CC([N+](=O)[O-])C1] |
| II-42 | 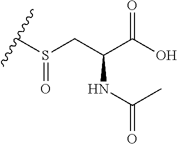 [SMILES: *S(=O)C[C@H](NC(C)=O)C(=O)O] | 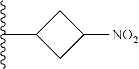 [SMILES: *C1CC([N+](=O)[O-])C1] |
| II-43 | 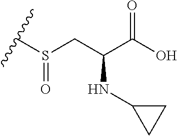 [SMILES: *S(=O)C[C@H](NC1CC1)C(=O)O] | 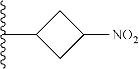 [SMILES: *C1CC([N+](=O)[O-])C1] |
| II-44 | 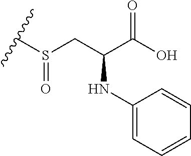 [SMILES: *S(=O)C[C@H](Nc1ccccc1)C(=O)O] | 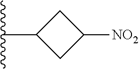 [SMILES: *C1CC([N+](=O)[O-])C1] |
| II-45 | 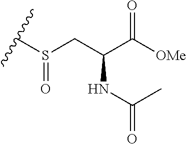 [SMILES: *S(=O)C[C@H](NC(C)=O)C(=O)OC] | 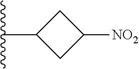 [SMILES: *C1CC([N+](=O)[O-])C1] |
| II-46 | 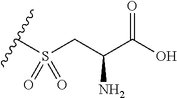 [SMILES: *S(=O)(=O)C[C@H](N)C(=O)O] | 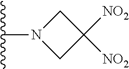 [SMILES: *N1CC([N+](=O)[O-])([N+](=O)[O-])C1] |
| II-47 | 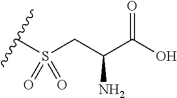 [SMILES: *S(=O)(=O)C[C@H](N)C(=O)O] | 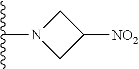 [SMILES: *N1CC([N+](=O)[O-])C1] |
| II-48 | 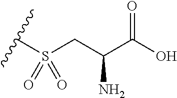 [SMILES: *S(=O)(=O)C[C@H](N)C(=O)O] | 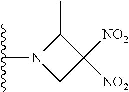 [SMILES: *N1CC([N+](=O)[O-])([N+](=O)[O-])C1C] |
| II-49 | 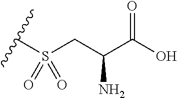 [SMILES: *S(=O)(=O)C[C@H](N)C(=O)O] | 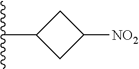 [SMILES: *C1CC([N+](=O)[O-])C1] |
| II-50 | 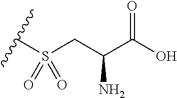 [SMILES: *S(=O)(=O)C[C@H](N)C(=O)O] | 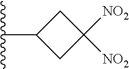 [SMILES: *C1CC([N+](=O)[O-])([N+](=O)[O-])C1] |
| II-51 | 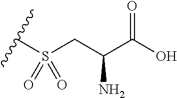 [SMILES: *S(=O)(=O)C[C@H](N)C(=O)O] | 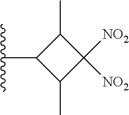 [SMILES: *C1C(C)C([N+](=O)[O-])([N+](=O)[O-])C1C] |
| II-52 | 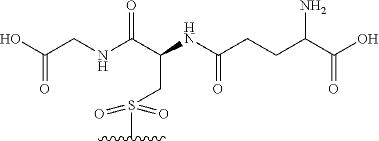 [SMILES: *S(=O)(=O)C[C@H](NC(=O)CCC(N)C(=O)O)C(=O)NCC(=O)O] | 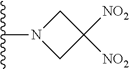 [SMILES: *N1CC([N+](=O)[O-])([N+](=O)[O-])C1] |
| II-53 | 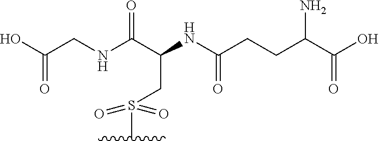 [SMILES: *S(=O)(=O)C[C@H](NC(=O)CCC(N)C(=O)O)C(=O)NCC(=O)O] | 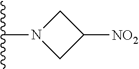 [SMILES: *N1CC([N+](=O)[O-])C1] |
| II-54 | 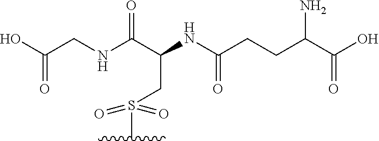 [SMILES: *S(=O)(=O)C[C@H](NC(=O)CCC(N)C(=O)O)C(=O)NCC(=O)O] | 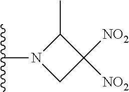 [SMILES: *N1CC([N+](=O)[O-])([N+](=O)[O-])C1C] |
| II-55 | 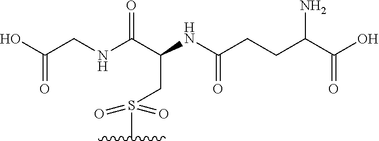 [SMILES: *S(=O)(=O)C[C@H](NC(=O)CCC(N)C(=O)O)C(=O)NCC(=O)O] | 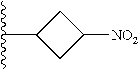 [SMILES: *C1CC([N+](=O)[O-])C1] |

TABLE 2

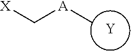

| Compound | | | |
|---|---|---|---|
| No. | X | A | Y |
| III-1 | 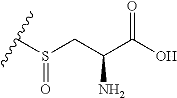 [SMILES: *S(=O)C[C@H](N)C(=O)O] | —CH₂C(O)— | 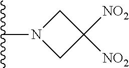 [SMILES: *N1CC([N+](=O)[O-])([N+](=O)[O-])C1] |
| III-2 | 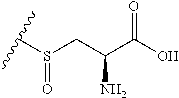 [SMILES: *S(=O)C[C@H](N)C(=O)O] | —CH₂C(O)— | 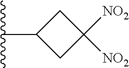 [SMILES: *C1CC([N+](=O)[O-])([N+](=O)[O-])C1] |
| III-3 | 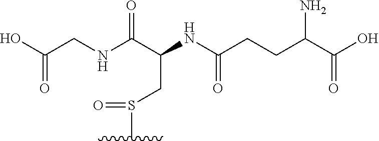 [SMILES: *S(=O)C[C@H](NC(=O)CCC(N)C(=O)O)C(=O)NCC(=O)O] | —CH₂C(O)— | 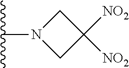 [SMILES: *N1CC([N+](=O)[O-])([N+](=O)[O-])C1] |
| III-4 | 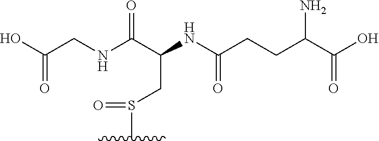 [SMILES: *S(=O)C[C@H](NC(=O)CCC(N)C(=O)O)C(=O)NCC(=O)O] | —CH₂C(O)— | 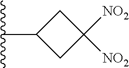 [SMILES: *C1CC([N+](=O)[O-])([N+](=O)[O-])C1] |
| III-5 | 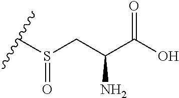 [SMILES: *S(=O)C[C@H](N)C(=O)O] | —C(O)CH₂CH₂— | 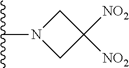 [SMILES: *N1CC([N+](=O)[O-])([N+](=O)[O-])C1] |
| III-6 | 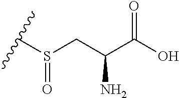 [SMILES: *S(=O)C[C@H](N)C(=O)O] | —C(O)CH₂CH₂— | 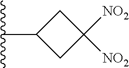 [SMILES: *C1CC([N+](=O)[O-])([N+](=O)[O-])C1] |
| III-7 | 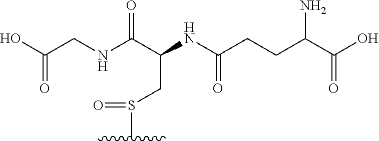 [SMILES: *S(=O)C[C@H](NC(=O)CCC(N)C(=O)O)C(=O)NCC(=O)O] | —C(O)CH₂CH₂— | 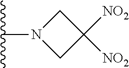 [SMILES: *N1CC([N+](=O)[O-])([N+](=O)[O-])C1] |
| III-8 | 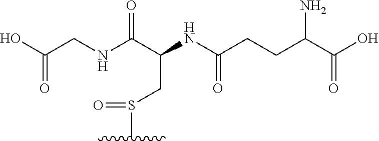 [SMILES: *S(=O)C[C@H](NC(=O)CCC(N)C(=O)O)C(=O)NCC(=O)O] | —C(O)CH₂CH₂— | 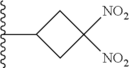 [SMILES: *C1CC([N+](=O)[O-])([N+](=O)[O-])C1] |
| III-9 | 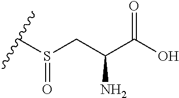 [SMILES: *S(=O)C[C@H](N)C(=O)O] | —CH₂— | 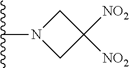 [SMILES: *N1CC([N+](=O)[O-])([N+](=O)[O-])C1] |
| III-10 | 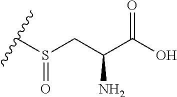 [SMILES: *S(=O)C[C@H](N)C(=O)O] | —CH₂— | 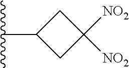 [SMILES: *C1CC([N+](=O)[O-])([N+](=O)[O-])C1] |
| III-11 | 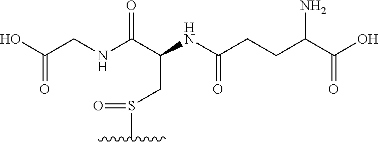 [SMILES: *S(=O)C[C@H](NC(=O)CCC(N)C(=O)O)C(=O)NCC(=O)O] | —CH₂— | 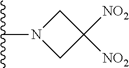 [SMILES: *N1CC([N+](=O)[O-])([N+](=O)[O-])C1] |
| III-12 | 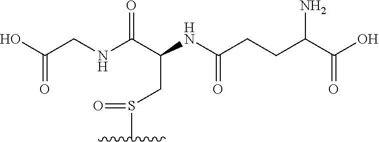 [SMILES: *S(=O)C[C@H](NC(=O)CCC(N)C(=O)O)C(=O)NCC(=O)O] | —CH₂— | 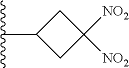 [SMILES: *C1CC([N+](=O)[O-])([N+](=O)[O-])C1] |
| III-13 | 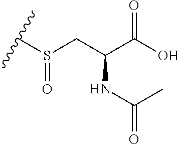 [SMILES: *S(=O)C[C@H](NC(C)=O)C(=O)O] | —CH₂C(O)— | 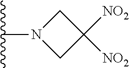 [SMILES: *N1CC([N+](=O)[O-])([N+](=O)[O-])C1] |
| III-14 | 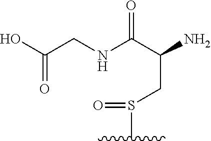 [SMILES: *S(=O)C[C@H](N)C(=O)NCC(=O)O] | —CH₂C(O)— | 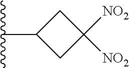 [SMILES: *C1CC([N+](=O)[O-])([N+](=O)[O-])C1] |
| III-15 | 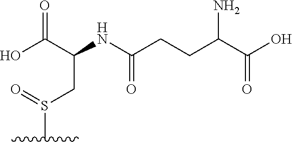 [SMILES: *S(=O)C[C@H](NC(=O)CCC(N)C(=O)O)C(=O)O] | —CH₂C(O)— | 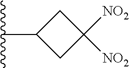 [SMILES: *C1CC([N+](=O)[O-])([N+](=O)[O-])C1] |
| III-16 | 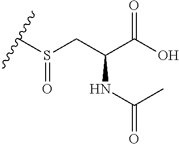 [SMILES: *S(=O)C[C@H](NC(C)=O)C(=O)O] | —C(O)CH₂CH₂— | 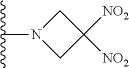 [SMILES: *N1CC([N+](=O)[O-])([N+](=O)[O-])C1] |
| III-17 | 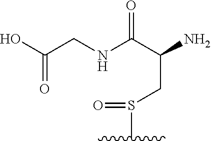 [SMILES: *S(=O)C[C@H](N)C(=O)NCC(=O)O] | —C(O)CH₂CH₂— | 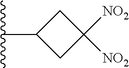 [SMILES: *C1CC([N+](=O)[O-])([N+](=O)[O-])C1] |
| III-18 | 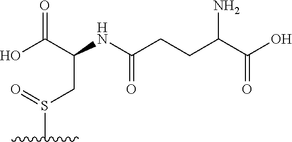 [SMILES: *S(=O)C[C@H](NC(=O)CCC(N)C(=O)O)C(=O)O] | —C(O)CH₂CH₂— | 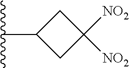 [SMILES: *C1CC([N+](=O)[O-])([N+](=O)[O-])C1] |
| III-19 | 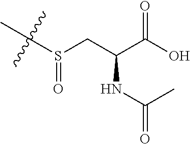 [SMILES: *S(=O)C[C@H](NC(C)=O)C(=O)O] | —CH₂— | 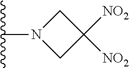 [SMILES: *N1CC([N+](=O)[O-])([N+](=O)[O-])C1] |
| III-20 | 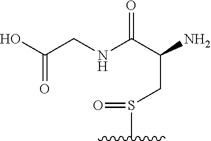 [SMILES: *S(=O)C[C@H](N)C(=O)NCC(=O)O] | —CH₂— | 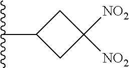 [SMILES: *C1CC([N+](=O)[O-])([N+](=O)[O-])C1] |
| III-21 | 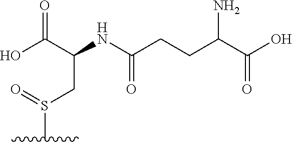 [SMILES: *S(=O)C[C@H](NC(=O)CCC(N)C(=O)O)C(=O)O] | —CH₂— | 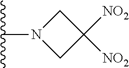 [SMILES: *N1CC([N+](=O)[O-])([N+](=O)[O-])C1] |
| III-22 | 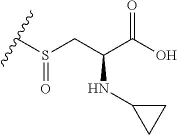 [SMILES: *S(=O)C[C@H](NC1CC1)C(=O)O] | —CH₂C(O)— | 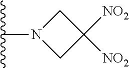 [SMILES: *N1CC([N+](=O)[O-])([N+](=O)[O-])C1] |
| III-23 | 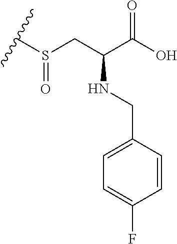 [SMILES: *S(=O)C[C@H](NCc1ccc(F)cc1)C(=O)O] | —C(O)CH₂CH₂— | 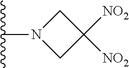 [SMILES: *N1CC([N+](=O)[O-])([N+](=O)[O-])C1] |
| III-24 | 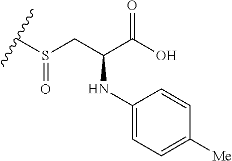 [SMILES: *S(=O)C[C@H](Nc1ccc(C)cc1)C(=O)O] | —CH₂— | 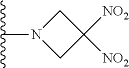 [SMILES: *N1CC([N+](=O)[O-])([N+](=O)[O-])C1] |
| III-25 | 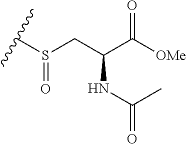 [SMILES: *S(=O)C[C@H](NC(C)=O)C(=O)OC] | —CH₂C(O)— | 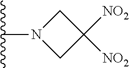 [SMILES: *N1CC([N+](=O)[O-])([N+](=O)[O-])C1] |
| III-26 | 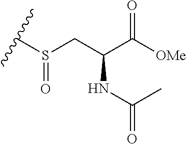 [SMILES: *S(=O)C[C@H](NC(C)=O)C(=O)OC] | —C(O)CH₂CH₂— | 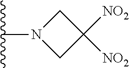 [SMILES: *N1CC([N+](=O)[O-])([N+](=O)[O-])C1] |
| III-27 | 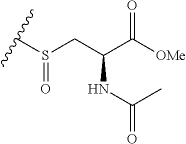 [SMILES: *S(=O)C[C@H](NC(C)=O)C(=O)OC] | —CH₂— | 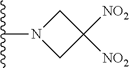 [SMILES: *N1CC([N+](=O)[O-])([N+](=O)[O-])C1] |
| III-28 | 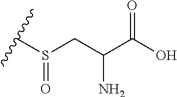 [SMILES: *S(=O)CC(N)C(=O)O] | —CH₂C(O)— | 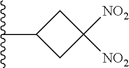 [SMILES: *C1CC([N+](=O)[O-])([N+](=O)[O-])C1] |
| III-29 | 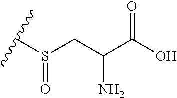 [SMILES: *S(=O)CC(N)C(=O)O] | —CH₂C(O)— | 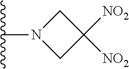 [SMILES: *N1CC([N+](=O)[O-])([N+](=O)[O-])C1] |
| III-30 | 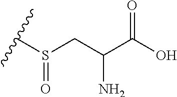 [SMILES: *S(=O)CC(N)C(=O)O] | —CH₂C(O)— | 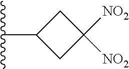 [SMILES: *C1CC([N+](=O)[O-])([N+](=O)[O-])C1] |
| III-31 | 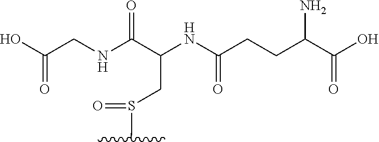 [SMILES: *S(=O)CC(NC(=O)CCC(N)C(=O)O)C(=O)NCC(=O)O] | —CH₂C(O)— | 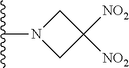 [SMILES: *N1CC([N+](=O)[O-])([N+](=O)[O-])C1] |
| III-32 | 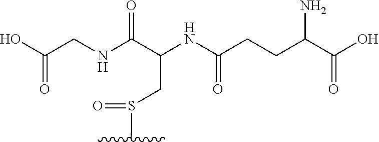 [SMILES: *S(=O)CC(NC(=O)CCC(N)C(=O)O)C(=O)NCC(=O)O] | —CH₂C(O)— | 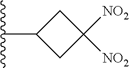 [SMILES: *C1CC([N+](=O)[O-])([N+](=O)[O-])C1] |
| III-33 | 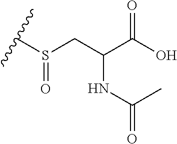 [SMILES: *S(=O)CC(NC(C)=O)C(=O)O] | —C(O)CH₂CH₂— | 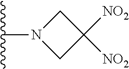 [SMILES: *N1CC([N+](=O)[O-])([N+](=O)[O-])C1] |
| III-34 | 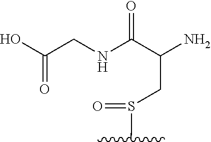 [SMILES: *S(=O)CC(N)C(=O)NCC(=O)O] | —CH₂— | 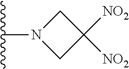 [SMILES: *N1CC([N+](=O)[O-])([N+](=O)[O-])C1] |
| III-35 | 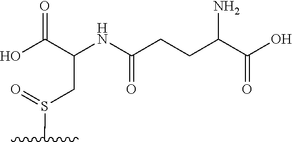 [SMILES: *S(=O)CC(NC(=O)CCC(N)C(=O)O)C(=O)O] | —CH₂C(O)— | 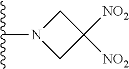 [SMILES: *N1CC([N+](=O)[O-])([N+](=O)[O-])C1] |
| III-36 | 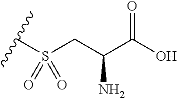 [SMILES: *S(=O)(=O)C[C@H](N)C(=O)O] | —CH₂C(O)— | 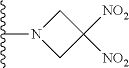 [SMILES: *N1CC([N+](=O)[O-])([N+](=O)[O-])C1] |
| III-37 | 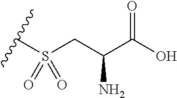 [SMILES: *S(=O)(=O)C[C@H](N)C(=O)O] | —CH₂C(O)— | 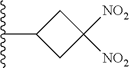 [SMILES: *C1CC([N+](=O)[O-])([N+](=O)[O-])C1] |
| III-38 | 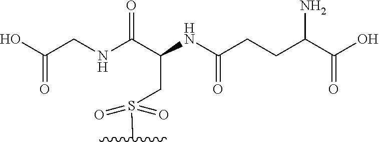 [SMILES: *S(=O)(=O)C[C@H](NC(=O)CCC(N)C(=O)O)C(=O)NCC(=O)O] | —CH₂C(O)— | 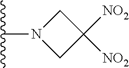 [SMILES: *N1CC([N+](=O)[O-])([N+](=O)[O-])C1] |
| III-39 | 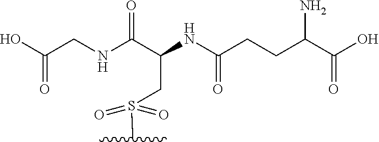 [SMILES: *S(=O)(=O)C[C@H](NC(=O)CCC(N)C(=O)O)C(=O)NCC(=O)O] | —CH₂C(O)— | 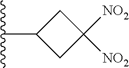 [SMILES: *C1CC([N+](=O)[O-])([N+](=O)[O-])C1] |
| III-40 | 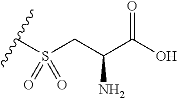 [SMILES: *S(=O)(=O)C[C@H](N)C(=O)O] | —C(O)CH₂CH₂— | 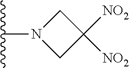 [SMILES: *N1CC([N+](=O)[O-])([N+](=O)[O-])C1] |
| III-41 | 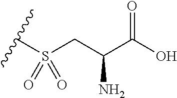 [SMILES: *S(=O)(=O)C[C@H](N)C(=O)O] | —C(O)CH₂CH₂— | 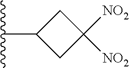 [SMILES: *C1CC([N+](=O)[O-])([N+](=O)[O-])C1] |
| III-42 | 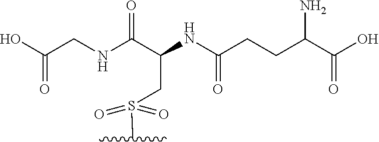 [SMILES: *S(=O)(=O)C[C@H](NC(=O)CCC(N)C(=O)O)C(=O)NCC(=O)O] | —C(O)CH₂CH₂— | 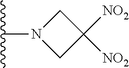 [SMILES: *N1CC([N+](=O)[O-])([N+](=O)[O-])C1] |
| III-43 | 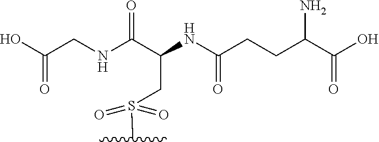 [SMILES: *S(=O)(=O)C[C@H](NC(=O)CCC(N)C(=O)O)C(=O)NCC(=O)O] | —C(O)CH₂CH₂— | 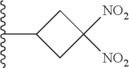 [SMILES: *C1CC([N+](=O)[O-])([N+](=O)[O-])C1] |
| III-44 | 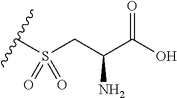 [SMILES: *S(=O)(=O)C[C@H](N)C(=O)O] | —CH₂— | 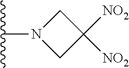 [SMILES: *N1CC([N+](=O)[O-])([N+](=O)[O-])C1] |
| III-45 | 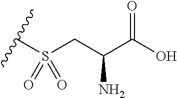 [SMILES: *S(=O)(=O)C[C@H](N)C(=O)O] | —CH₂— | 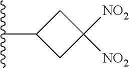 [SMILES: *C1CC([N+](=O)[O-])([N+](=O)[O-])C1] |

TABLE 3

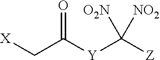

| Compound | | | |
|---|---|---|---|
| No. | X | Y | Z |
| IV-1 | 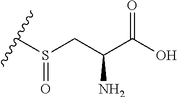 [SMILES: *S(=O)C[C@H](N)C(=O)O] | —N(H)CH₂— | methyl |
| IV-2 | 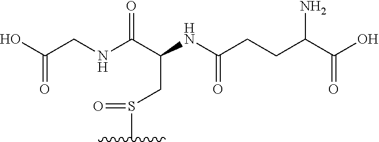 [SMILES: *S(=O)C[C@H](NC(=O)CCC(N)C(=O)O)C(=O)NCC(=O)O] | —N(H)CH₂— | methyl |
| IV-3 | 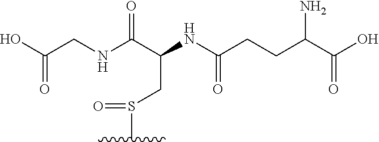 [SMILES: *S(=O)C[C@H](NC(=O)CCC(N)C(=O)O)C(=O)NCC(=O)O] | —N(H)CH₂— | ethyl |
| IV-4 | 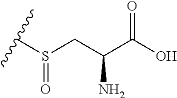 [SMILES: *S(=O)C[C@H](N)C(=O)O] | —N(H)CH₂— | n-penty1 |
| IV-5 | 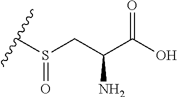 [SMILES: *S(=O)C[C@H](N)C(=O)O] | —N(H)CH₂— | hydrogen |
| IV-6 | 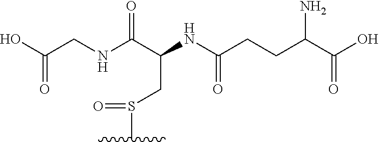 [SMILES: *S(=O)C[C@H](NC(=O)CCC(N)C(=O)O)C(=O)NCC(=O)O] | —N(H)CH₂CH₂— | methyl |
| IV-7 | 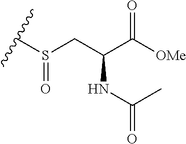 [SMILES: *S(=O)C[C@H](NC(C)=O)C(=O)OC] | —N(H)(CH₂)₄— | methyl |
| IV-8 | 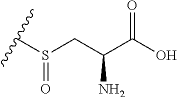 [SMILES: *S(=O)C[C@H](N)C(=O)O] | —N(CH₃)CH₂— | methyl |
| IV-9 | 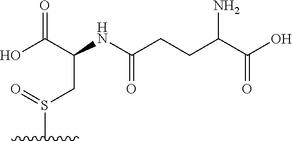 [SMILES: *S(=O)C[C@H](NC(=O)CCC(N)C(=O)O)C(=O)O] | —N(CH₃)(CH2)₃— | methyl |
| IV-10 | 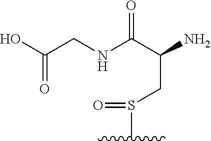 [SMILES: *S(=O)C[C@H](N)C(=O)NCC(=O)O] | —N(H)C(CH₃)(H)— | methyl |
| IV-11 | 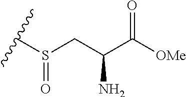 [SMILES: *S(=O)C[C@H](N)C(=O)OC] | —N(H)C(CH₃)(H)CH₂— | methyl |
| IV-12 | 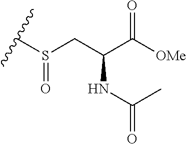 [SMILES: *S(=O)C[C@H](NC(C)=O)C(=O)OC] | —CH₂— | methyl |
| IV-13 | 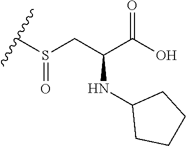 [SMILES: *S(=O)C[C@H](NC1CCCC1)C(=O)O] | —(CH2)₂— | methyl |
| IV-14 | 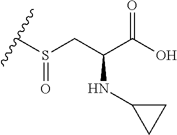 [SMILES: *S(=O)C[C@H](NC1CC1)C(=O)O] | —CH₂— | ethyl |
| IV-15 | 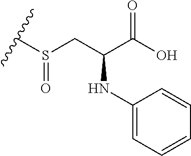 [SMILES: *S(=O)C[C@H](Nc1ccccc1)C(=O)O] | —(CH₂)₄— | isopropyl |
| IV-16 | 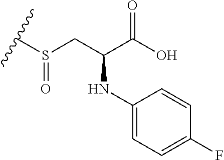 [SMILES: *S(=O)C[C@H](Nc1ccc(F)cc1)C(=O)O] | —(CH2)₂— | n-pentyl |
| IV-17 | 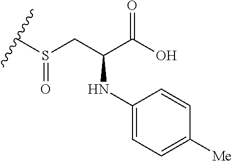 [SMILES: *S(=O)C[C@H](Nc1ccc(C)cc1)C(=O)O] | —CH₂— | hydrogen |
| IV-18 | 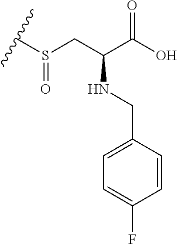 [SMILES: *S(=O)C[C@H](NCc1ccc(F)cc1)C(=O)O] | —CH₂CH₂C(CH₃)₂— | methyl |
| IV-19 | 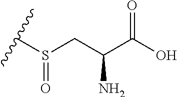 [SMILES: *S(=O)C[C@H](N)C(=O)O] | —CH₂C(CH₃)₂CH₂— | methyl |
| IV-20 | 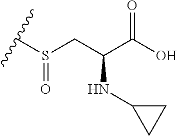 [SMILES: *S(=O)C[C@H](NC1CC1)C(=O)O] | —N(H)CH₂— | methyl |
| IV-21 | 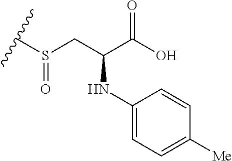 [SMILES: *S(=O)C[C@H](Nc1ccc(C)cc1)C(=O)O] | —N(H)CH₂— | ethyl |
| IV-22 | 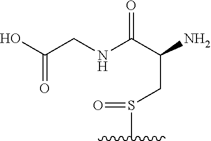 [SMILES: *S(=O)C[C@H](N)C(=O)NCC(=O)O] | —N(CH₃)CH₂— | methyl |
| IV-23 | 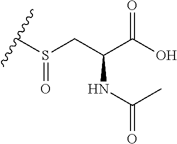 [SMILES: *S(=O)C[C@H](NC(C)=O)C(=O)O] | —(CH2)₂— | methyl |
| IV-24 | 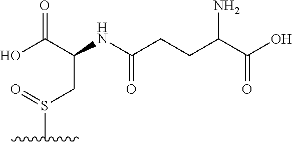 [SMILES: *S(=O)C[C@H](NC(=O)CCC(N)C(=O)O)C(=O)O] | —N(H)CH₂— | methyl |
| IV-25 | 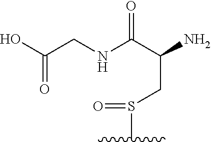 [SMILES: *S(=O)C[C@H](N)C(=O)NCC(=O)O] | —N(H)CH₂— | ethyl |
| IV-26 | 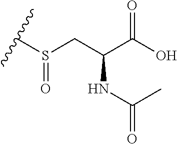 [SMILES: *S(=O)C[C@H](NC(C)=O)C(=O)O] | —N(CH₃)CH₂— | methyl |
| IV-27 | 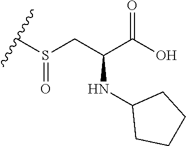 [SMILES: *S(=O)C[C@H](NC1CCCC1)C(=O)O] | —(CH₂)₂— | methyl |
| IV-28 | 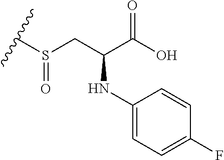 [SMILES: *S(=O)C[C@H](Nc1ccc(F)cc1)C(=O)O] | —N(H)CH₂— | methyl |
| IV-29 | 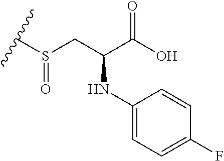 [SMILES: *S(=O)C[C@H](Nc1ccc(F)cc1)C(=O)O] | —N(CH3)CH₂— | methyl |
| IV-30 | 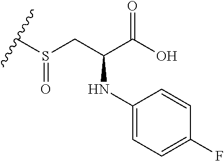 [SMILES: *S(=O)C[C@H](Nc1ccc(F)cc1)C(=O)O] | —N(H)CH₂— | methyl |
| IV-31 | 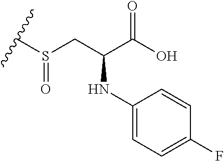 [SMILES: *S(=O)C[C@H](Nc1ccc(F)cc1)C(=O)O] | —N(CH₃)CH₂— | methyl |
| IV-32 | 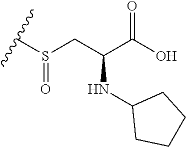 [SMILES: *S(=O)C[C@H](NC1CCCC1)C(=O)O] | —N(H)CH₂— | methyl |
| IV-33 | 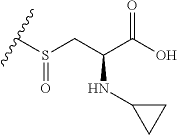 [SMILES: *S(=O)C[C@H](NC1CC1)C(=O)O] | —N(CH₃)CH₂— | methyl |
| IV-34 | 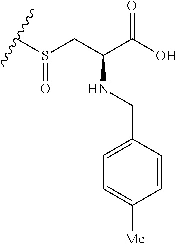 [SMILES: *S(=O)C[C@H](NCc1ccc(C)cc1)C(=O)O] | —N(H)CH₂— | methyll= |
| IV-35 | 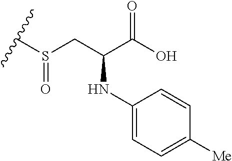 [SMILES: *S(=O)C[C@H](Nc1ccc(C)cc1)C(=O)O] | —N(CH₃)CH₂— | methyl |
| IV-36 | 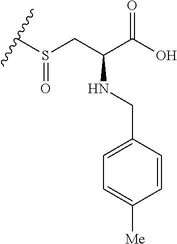 [SMILES: *S(=O)C[C@H](NCc1ccc(C)cc1)C(=O)O] | —(CH₂)₂— | methyl |
| IV-37 | 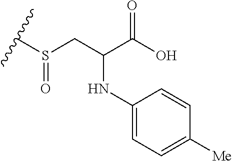 [SMILES: *S(=O)CC(Nc1ccc(C)cc1)C(=O)O] | —N(H)CH₂— | n-pentyl |
| IV-38 | 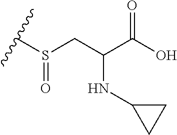 [SMILES: *S(=O)CC(NC1CC1)C(=O)O] | —N(CH₃)CH₂— | methyl |
| IV-39 | 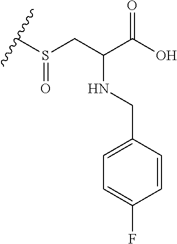 [SMILES: *S(=O)CC(NCc1ccc(F)cc1)C(=O)O] | —N(H)CH₂CH₂— | ethyl |
| IV-40 | 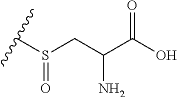 [SMILES: *S(=O)CC(N)C(=O)O] | —N(H)CH₂— | methyl |
| IV-41 | 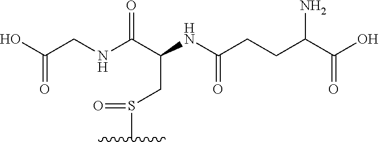 [SMILES: *S(=O)C[C@H](NC(=O)CCC(N)C(=O)O)C(=O)NCC(=O)O] | —N(H)CH₂— | methyl |
| IV-42 | 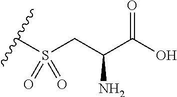 [SMILES: *S(=O)(=O)C[C@H](N)C(=O)O] | —N(H)CH₂— | methyl |
| IV-43 | 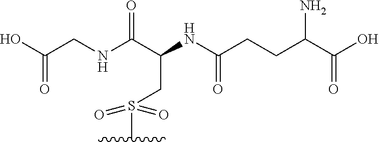 [SMILES: *S(=O)(=O)C[C@H](NC(=O)CCC(N)C(=O)O)C(=O)NCC(=O)O] | —N(H)CH₂— | methyl |
| IV-44 | 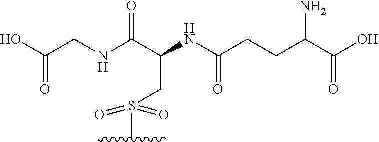 [SMILES: *S(=O)(=O)C[C@H](NC(=O)CCC(N)C(=O)O)C(=O)NCC(=O)O] | —N(H)CH₂— | ethyl |
| IV-45 | 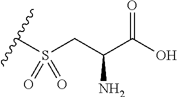 [SMILES: *S(=O)(=O)C[C@H](N)C(=O)O] | —N(H)CH₂— | n-penty1 |
| IV-46 | 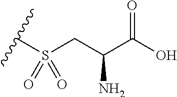 [SMILES: *S(=O)(=O)C[C@H](N)C(=O)O] | —N(H)CH₂— | hydrogen |
| IV-47 | 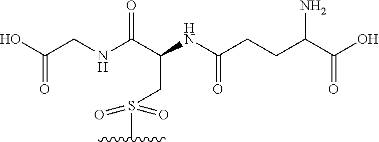 [SMILES: *S(=O)(=O)C[C@H](NC(=O)CCC(N)C(=O)O)C(=O)NCC(=O)O] | —N(H)CH₂CH₂— | methyl |
| IV-48 | 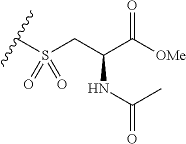 [SMILES: *S(=O)(=O)C[C@H](NC(C)=O)C(=O)OC] | —N(H)(CH₂)₄— | methyl |
| IV-49 | 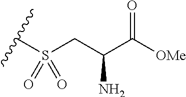 [SMILES: *S(=O)(=O)C[C@H](N)C(=O)OC] | —N(CH₃)CH₂— | methyl |
| IV-50 | 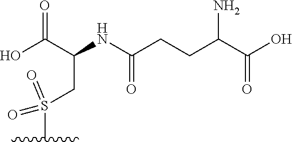 [SMILES: *S(=O)(=O)C[C@H](NC(=O)CCC(N)C(=O)O)C(=O)O] | —N(CH₃)(CH₂)₃— | methyl |
| IV-51 | 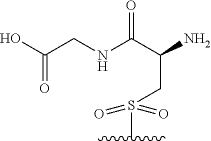 [SMILES: *S(=O)(=O)C[C@H](N)C(=O)NCC(=O)O] | —N(H)C(CH₃)(H)— | methyl |

Methods for preparing compounds are described for example, in U.S. Pat. No. 11,008,287, the disclosure of which is incorporated herein by reference. However, methods for making compounds the compounds in TABLES 1-3 are illustrated in the following synthetic schemes. These schemes are given for the purpose of illustrating the invention, and should not be regarded in any manner as limiting the scope or the spirit of the invention. Starting materials shown in the schemes can be obtained from commercial sources or can be prepared based on procedures described in the literature.

The synthetic route illustrated in Scheme 1 depicts a general method for preparing cyclic geminal di-nitro compounds. In the first step, chloro epoxide A1 is reacted with t-butylamine to provide hydroxy heterocyclic compound B1. Mesylation of the hydroxyl group of heterocyclic compound B1 with methylsulfonyl chloride gives mesylate C1, which upon reacting with NaNO₂ generates cyclic mono-nitro compound D1. Further nitration of compound D1 can be carried out using NaNO₂ in the presence of Na₂S₂O₈ and K₃Fe(CN)₆ to provide geminal di-nitro heterocyclic compound E1. A three-step procedure provides thioether compound G1, which involves reaction of compound E1 with boron trifluoride etherate, acylation with acetyl bromide F, and thiolation to provide compound G1. Subjecting thioether G1 to oxidation conditions can produce sulfoxide X1 and sulfone Y1. Procedures for converting a thioether to a sulfoxide or sulfone using oxidizing conditions can be found in the literature. Further description of related synthetic procedures are described in, for example, Archibald et al. in J. Org. Chem. 1990, 55, 2920-2924; U.S. Pat. No. 7,507,842; and J. P. Agrawal, R. D. Hodgson, Organic Chemistry of Explosives, Wiley & Sons, England, 2007 and references cited therein.

This synthetic procedure illustrated in Scheme 1 and described above is contemplated to be applicable to preparing compounds having various substituents at the R₁, R₂, R₃ and R₄ positions. If a particular epoxide compound embraced by A1 should contain a functional group sensitive to one or more of the synthetic transformations in Scheme 1, then standard protecting group strategies are contemplated to be applied. For further description of protecting group strategies and procedures, see, for example, Greene, T. W.; Wuts, P. G. M. Protective Groups in Organic Synthesis, 2^{nd} ed.; Wiley, New York, 1991.

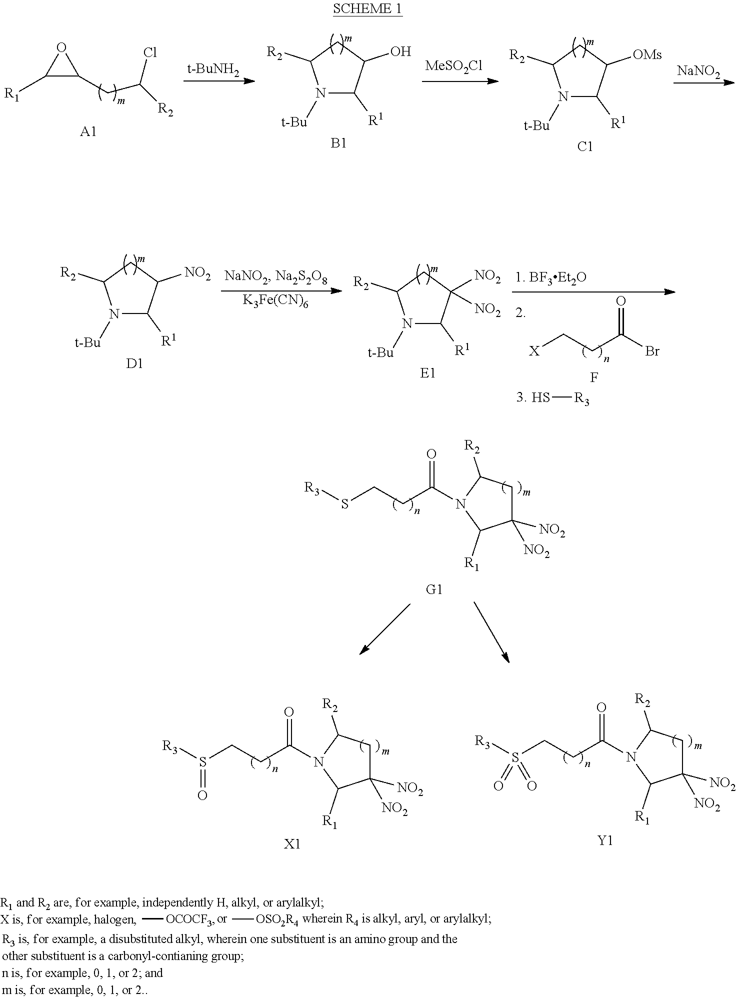

Scheme 2 illustrates a more specific embodiment of the synthetic route shown in Scheme 1 when m is 0. In the first step, epoxide A2 is reacted with t-butylamine to provide hydroxyl azetidine B2. Mesylation of the hydroxyl group of azetidine B2 with methylsulfonyl chloride gives azetidine mesylate C2, which upon reacting with NaNO₂ generates mono-nitro azetidine D2. Alternatively, the mono-nitro azetidine can be trapped with formaldehyde to provide a more stable product. Further nitration of mono-nitro azetidine D2 with NaNO₂ in the presence of Na₂S₂O₈ and K₃Fe(CN)₆ furnishes the geminal di-nitro azetidine E2. If the formaldehyde trapped product is used, the reaction can be carried out under basic conditions to release the mono-nitro azetidine in situ. A four-step procedure provides di-nitro azetidines X2 and Y2, which involves reaction of compound E2 with boron trifluoride etherate, acylation with acetyl bromide F, thiolation to provide di-nitro azetidine G2, and then oxidation to provide sulfoxide X2 and sulfone Y2. Procedures for converting a thioether to a sulfoxide or sulfone using oxidizing conditions can be found in the literature. This synthetic procedure is contemplated to be applicable to preparing compounds having various substituents at the R₁, R₂, R₃ and R₄ positions. If a particular epoxide compound embraced by A2 should contain a functional group sensitive to one or more of the synthetic transformations in Scheme 2, then standard protecting group strategies are contemplated to be applied. For further description of protecting group strategies and procedures, see, for example, Greene, T. W.; Wuts, P. G. M. Protective Groups in Organic Synthesis, 2^{nd} ed.; Wiley, New York, 1991. Furthermore, mono-nitro compounds can be prepared by treating mono-nitro compound D2 with a Lewis Acid (e.g., boron trifluoride etherate) and acetyl bromide compound F (e.g., from Scheme 2) to provide the desired mono-nitro product.

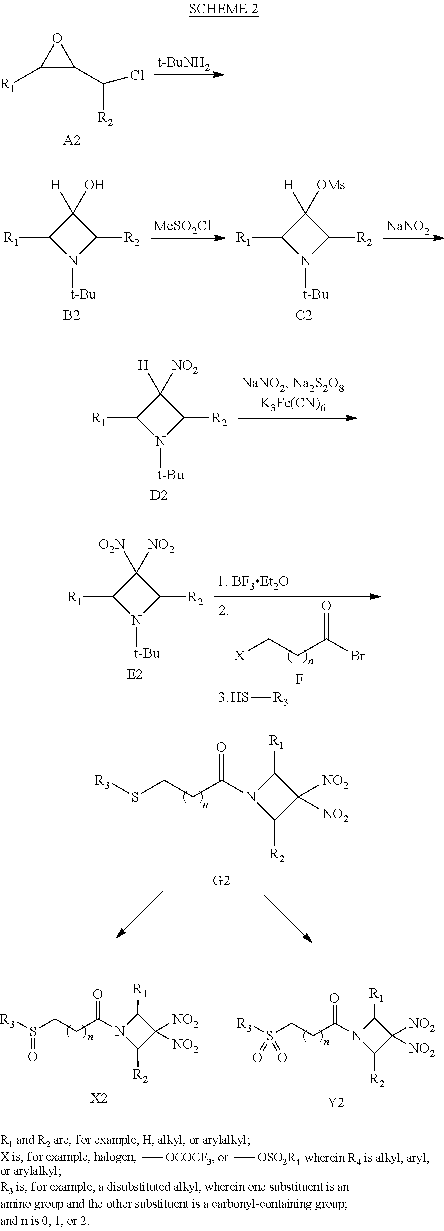

Scheme 3 illustrates another more particular embodiment of the synthetic route shown in Scheme 1 when both R₁ and R₂ are hydrogen and m is 0. In the first step, commercially available epichlorohydrin A3 is reacted with t-butylamine to provide hydroxyl azetidine B3. Mesylation of the hydroxyl group of azetidine B3 with methylsulfonyl chloride gives azetidine mesylate C3, which upon reacting with NaNO₂ generates mono-nitro azetidine D3. Further nitration of mono-nitro azetidine D3 with NaNO₂ in the presence of Na₂S₂O₈ and K₃Fe(CN)₆ furnishes the geminal di-nitro azetidine E3. A four-step procedure provides di-nitro azetidines X3 and Y3, which involves reaction of compound E3 with boron trifluoride etherate, acylation with acetyl bromide, and thiolation to provide di-nitro azetidine F3. Oxidation of thioether F3 can provide sulfoxide X3 and sulfone Y3. Procedures for converting a thioether to a sulfoxide or sulfone using oxidizing conditions can be found in the literature. Further description of related synthetic procedures are described in, for example, Archibald et al. in J. Org. Chem. 1990, 55, 2920-2924; U.S. Pat. No. 7,507,842; and J. P. Agrawal, R. D. Hodgson, Organic Chemistry of Explosives, Wiley & Sons, England, 2007 and references cited therein. Furthermore, mono-nitro compounds can be prepared by treating mono-nitro compound D3 with a Lewis Acid (e.g., boron trifluoride etherate) and acetyl bromide compound F to provide the desired bromo mono-nitro product, which may be subjected to debromination procedures to replace the bromine atom with a hydrogen.

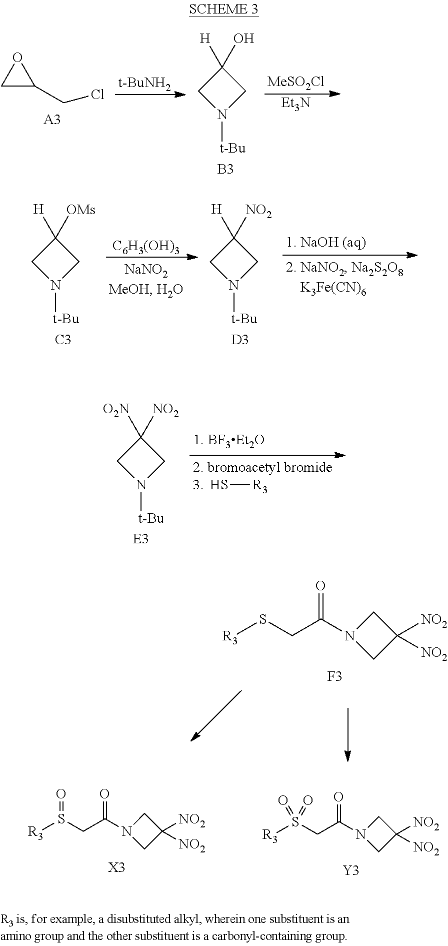

Scheme 4 illustrates an alternative exemplary procedure for preparing cyclic geminal di-nitro compounds. In the first step, heterocyclic compound A4 is reacted with an oxidant, such as pyridinium dichromate (PDC), to provide heterocyclic ketone B4. Reaction of ketone B4 with hydroxylamine gives heterocyclic oxime C4, which upon reaction with N-bromosuccinimide (NBS) produces bromo nitro compound D4. Reaction of compound D4 with NaBH₄ furnishes mono-nitro compound E4. Reaction of mono-nitro compound E4 with NaNO₂ in the presence of Na₂S₂O₈ and K₃Fe(CN)₆ provides geminal di-nitro heterocyclic compound F4. A four-step procedure provides cyclic geminal di-nitro compounds X4 and Y4, which involves reaction of compound F4 with a deprotecting agent, acylation with acetyl bromide compound F, thiolation to provide cyclic geminal di-nitro product G4, then oxidation to provide sulfoxide X4 and sulfone Y4. Procedures for converting a thioether to a sulfoxide or sulfone using oxidizing conditions can be found in the literature. Further description of related synthetic procedures are described in, for example, Archibald et al. in J. Org. Chem. 1990, 55, 2920-2924; U.S. Pat. No. 7,507,842; and J. P. Agrawal, R. D. Hodgson, Organic Chemistry of Explosives, Wiley & Sons, England, 2007 and references cited therein. Furthermore, mono-nitro compounds can be prepared by treating mono-nitro compound D4 with a deprotecting agent and acetyl bromide compound F to provide the desired bromo mono-nitro product, which may be subjected to debromination procedures to replace the bromine atom with a hydrogen.

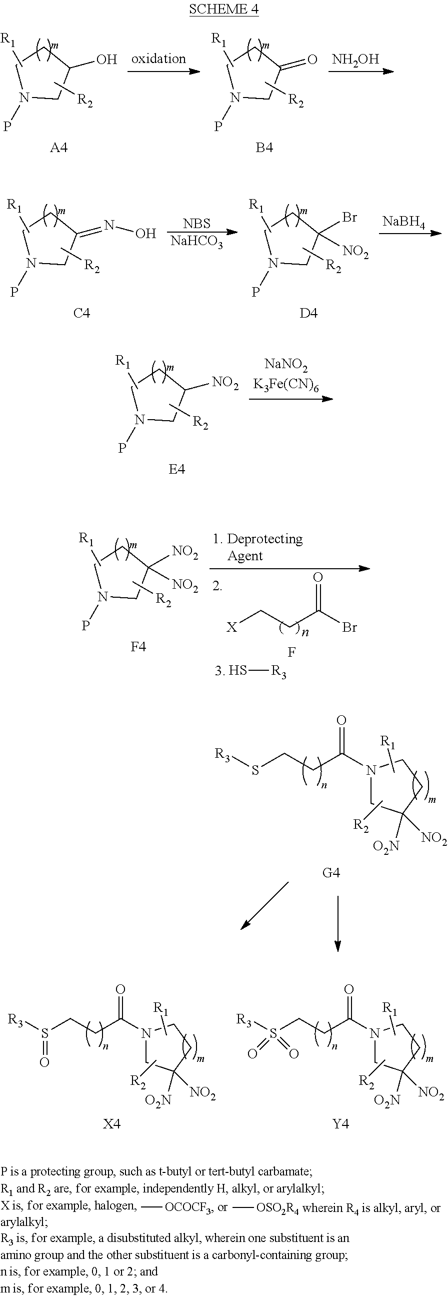

Scheme 5 illustrates yet another exemplary procedure for preparing cyclic geminal di-nitro compounds with initial steps different from those shown in Scheme 4. In the first step, heterocyclic compound A4 is reacted with methylsulfonyl chloride to provide heterocyclic mesylate B5. Reaction of mesylate B5 with NaNO₂ gives mono-nitro compound E4. Nitration of compound E4 with NaNO₂ in the presence of Na₂S₂O₈ and K₃Fe(CN)₆ provides geminal di-nitro compound F4. A three-step procedure provides di-nitro compound G4, which involves reaction of compound F4 with a deprotecting agent, acylation with acetyl bromide compound F, and thiolation to provide di-nitro compound G4. Oxidation of thioether G4 then provides sulfoxide X4 and sulfone Y4. Procedures for converting a thioether to a sulfoxide or sulfone using oxidizing conditions can be found in the literature. Further description of related synthetic procedures are described in, for example, Archibald el al. in J. Org. Chem. 1990, 55, 2920-2924: U.S. Pat. No. 7,507,842; and J. P. Agrawal, R. D. Hodgson, Organic Chemistry of Explosives, Wiley & Sons, England, 2007 and references cited therein. Furthermore, mono-nitro compounds can be prepared by treating mono-nitro compound E4 with a deprotecting agent and acetyl bromide compound F to provide the desired mono-nitro product.

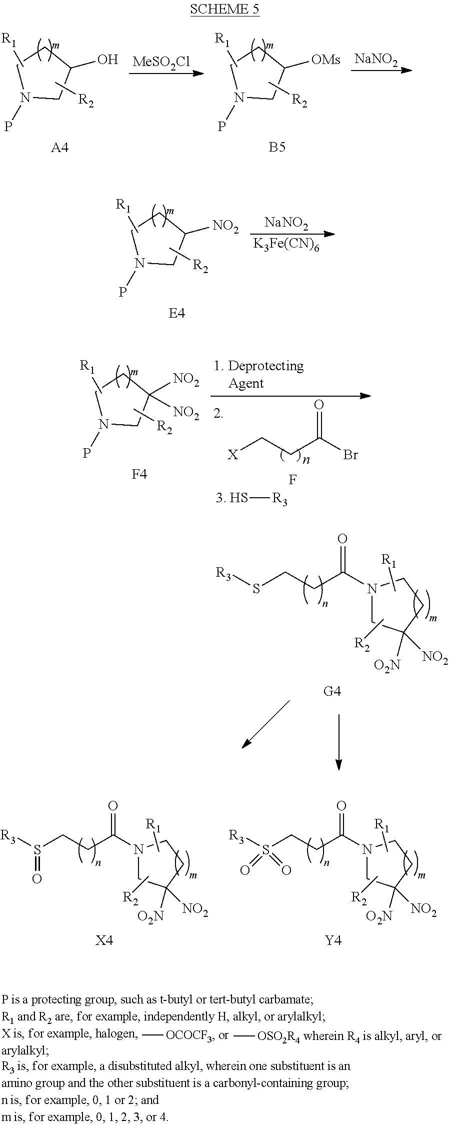

The synthetic route illustrated in Scheme 6 depicts an exemplary method for preparing cyclic vicinal di-nitro compounds. In the first step, cycloalkene A6 is reacted with N₂O₄ to provide vicinal di-nitro compound B6. A three-step procedure provides vicinal di-nitro product C6, which involves reaction of compound B6 with a deprotecting agent, acylation with acetyl bromide compound F, and thiolation to provide vicinal di-nitro compound C6. Oxidation of thioether C6 then provides sulfoxide X6 and sulfone Y6. Procedures for converting a thioether to a sulfoxide or sulfone using oxidizing conditions can be found in the literature. Further description of related synthetic procedures are described in, for example, Archibald et al. in J. Org. Chem. 1990, 55, 2920-2924; U.S. Pat. No. 7,507,842; and J. P. Agrawal, R. D. Hodgson, Organic Chemistry of Explosives, Wiley & Sons, England, 2007 and references cited therein. This synthetic procedure is contemplated to be applicable to preparing compounds having various substituents at the R₁, R₂, R₃ and R₄ positions. If a particular cycloalkene compound embraced by A6 should contain a functional group sensitive to one or more of the synthetic transformations in Scheme 6, then standard protecting group strategies are contemplated to be applied. For further description of protecting group strategies and procedures, see, for example, Greene. T. W.; Wuts, P. G. M. Protective Groups in Organic Synthesis, 2^{nd} ed.; Wiley, New York, 1991.

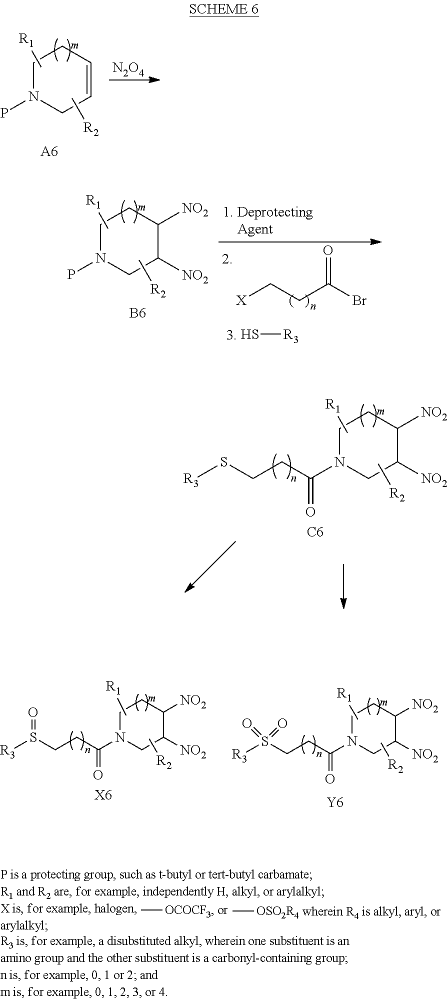

The synthetic route illustrated in Scheme 7 depicts a general method for preparing cyclic mono-nitro compounds. In the first step, chloro epoxide A7 is reacted with t-butylamine to provide hydroxy heterocyclic compound B7. Mesylation of the hydroxyl group of heterocyclic compound B7 with methylsulfonyl chloride gives mesylate C7 which upon reacting with NaNO₂ generates cyclic mono-nitro compound D7. A three-step procedure provides compound G7, which involves reaction of compound D7 with boron trifluoride etherate, acylation with acetyl bromide compound F, and thiolation to provide compound G7. Oxidation of thioether G7 then provides sulfoxide X7 and sulfone Y7. Procedures for converting a thioether to a sulfoxide or sulfone using oxidizing conditions can be found in the literature. Further description of related synthetic procedures are described in, for example, Archibald et al. in J. Org. Chem. 1990, 55, 2920-2924; U.S. Pat. No. 7,507,842; and J. P. Agrawal, R. D. Hodgson, Organic Chemistry of Explosives, Wiley & Sons, England, 2007 and references cited therein. This synthetic procedure illustrated in Scheme 7 is contemplated to be applicable to preparing compounds having various substituents at the R₁, R₂, R₃ and R₄ positions. If a particular epoxide compound embraced by A7 should contain a functional group sensitive to one or more of the synthetic transformations in Scheme 7, then standard protecting group strategies are contemplated to be applied. For further description of protecting group strategies and procedures, see, for example, Greene, T. W.; Wuts, P. G. M. Protective Groups in Organic Synthesis, 2^{nd} ed.; Wiley, New York, 1991.

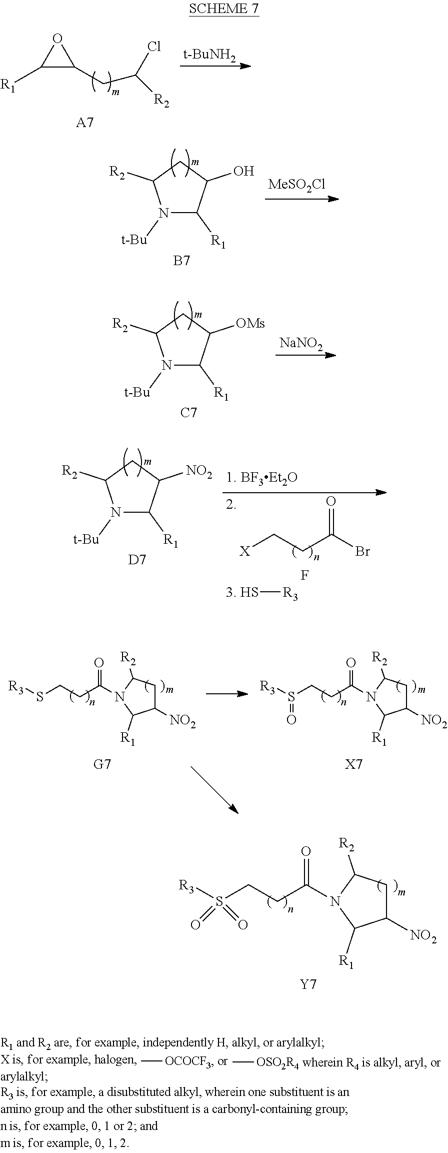

The synthetic routes described above can be modified to prepare compounds having an alkyl halide attached to the ring nitrogen atom. Exemplary synthetic procedures for preparing such compounds include reducing the amide group of compound G1-G4, G7, and C6 to an amine. Alternatively, compound F used in the procedures above could be replaced with an appropriately protected alkyl halide, such that after the alkylation reaction, the protected alkyl group attached to the ring nitrogen atom is deprotected and converted to an alkyl chloride or bromide.

Scheme 8 depicts another exemplary method for preparing cyclic mono-nitro and di-nitro compounds. Reaction of ketone B8 with hydroxylamine gives heterocyclic hydroxylamine C8, which upon reaction with N-bromosuccinimide (NBS) produces bromo nitro compound D8. Reaction of compound D8 with NaBH₄ furnishes mono-nitro compound E8. The hydroxyl protecting group (P, which may be, for example, a tert-butyldimethylsilyl group) and the 1,2-dihydroxyethane protecting group are removed using standard deprotection conditions. Exemplary deprotection conditions for removing a tert-butyldimethyl silyl group include addition of tetra-n-butylammonium fluoride. Exemplary deprotection conditions for removing a 1,2-dihydroxyethane protecting group include addition of hydrochloric acid and water. Hydroxy-ketone F8 can be converted to α-bromo ketone G8 by first reacting compound F8 with methanesulfonyl chloride to form a mesylate and then adding sodium bromide to form α-bromo ketone G8.

Di-nitro compounds can be prepared by reacting mono-nitro compound E8 with NaNO₂ in the presence of Na₂S₂O₈ and K₃Fe(CN)₆ to provide geminal di-nitro heterocyclic compound H8. The hydroxyl protecting group (P, which may be, for example, a tert-butyldimethyl silyl group) and the 1,2-dihydroxyethane protecting group of compound H8 may be removed using standard deprotection conditions. Exemplary deprotection conditions for removing a tert-butyldimethyl silyl group include addition of tetra-n-butylammonium fluoride. Exemplary deprotection conditions for removing a 1,2-dihydroxyethane protecting group include addition of hydrochloric acid and water. Hydroxy-ketone I8 can be converted to α-bromo ketone J8 by first reacting compound I8 with methanesulfonyl chloride to form a mesylate and then adding sodium bromide to form an α-bromo ketone. Thiolation of the α-bromo ketone provides the thioether J8. Oxidation of thioether J8 then provides sulfoxide X8 and sulfone Y8. Procedures for converting a thioether to a sulfoxide or sulfone using oxidizing conditions can be found in the literature. Further description of related synthetic procedures are described in, for example. Archibald et al. in J. Org. Chem. 1990, 55, 2920-2924 and J. P. Agrawal. R. D. Hodgson, Organic Chemistry of Explosives, Wiley & Sons, England, 2007 and references cited therein.

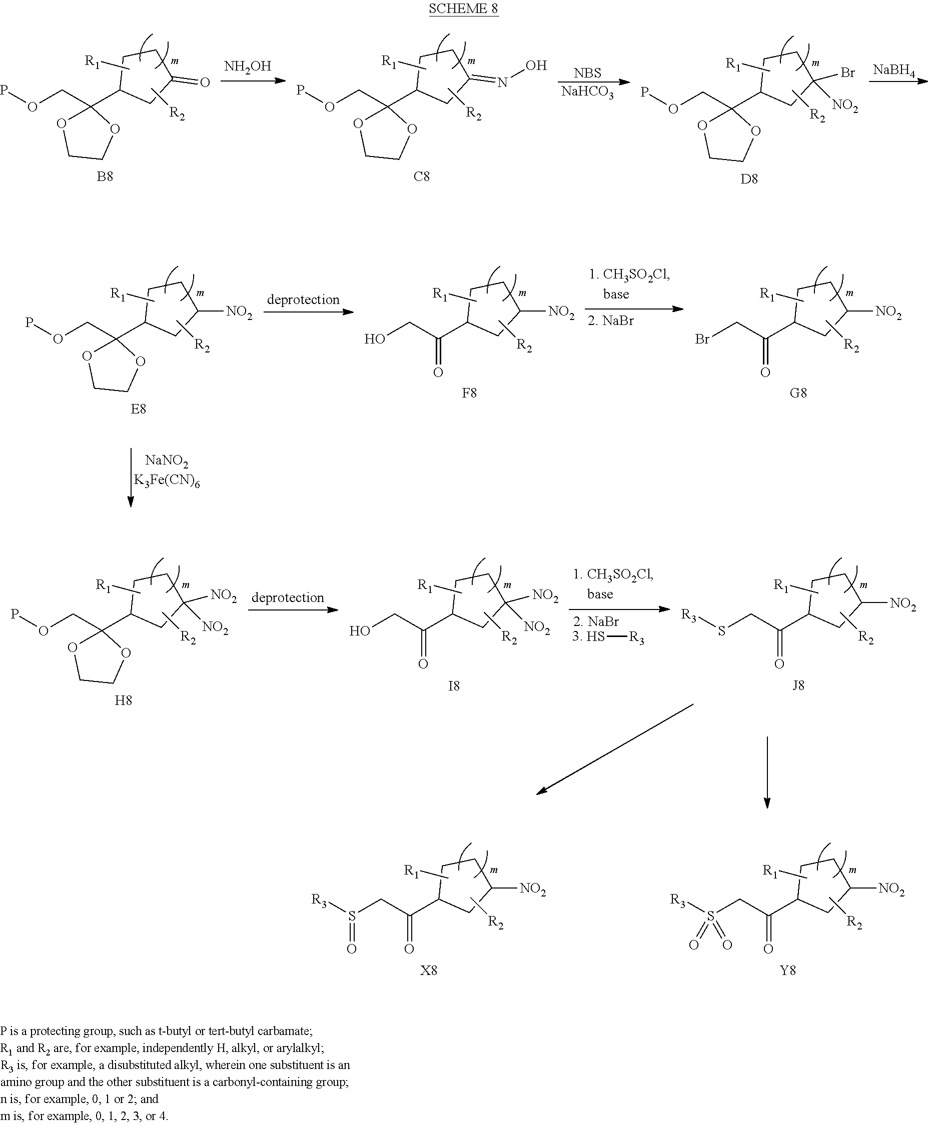

Scheme 9 illustrates an exemplary procedure for preparing acyclic geminal di-nitro compounds. In the first step, protected amino alcohol A9 is reacted with methylsulfonyl chloride to provide mesylate B9. Reaction of mesylate B9 with NaNO₂ gives mono-nitro compound E9. Nitration of compound E9 with NaNO₂ in the presence of Na₂S₂O₈ and K₃Fe(CN)₆ provides geminal di-nitro compound F9. A three-step procedure provides the desired di-nitro product G9, which involves reaction of compound F9 with a deprotecting agent, acylation with acetyl bromide compound F, and thiolation to provide di-nitro product G9. Oxidation of thioether G9 then provides sulfoxide X9 and sulfone Y9. Procedures for converting a thioether to a sulfoxide or sulfone using oxidizing conditions can be found in the literature. Further description of related synthetic procedures are described in, for example, Archibald et al. in J. Org. Chem. 1990, 55, 2920-2924; U.S. Pat. No. 7,507,842; and J. P. Agrawal, R. D. Hodgson, Organic Chemistry of Explosives, Wiley & Sons, England, 2007 and references cited therein.

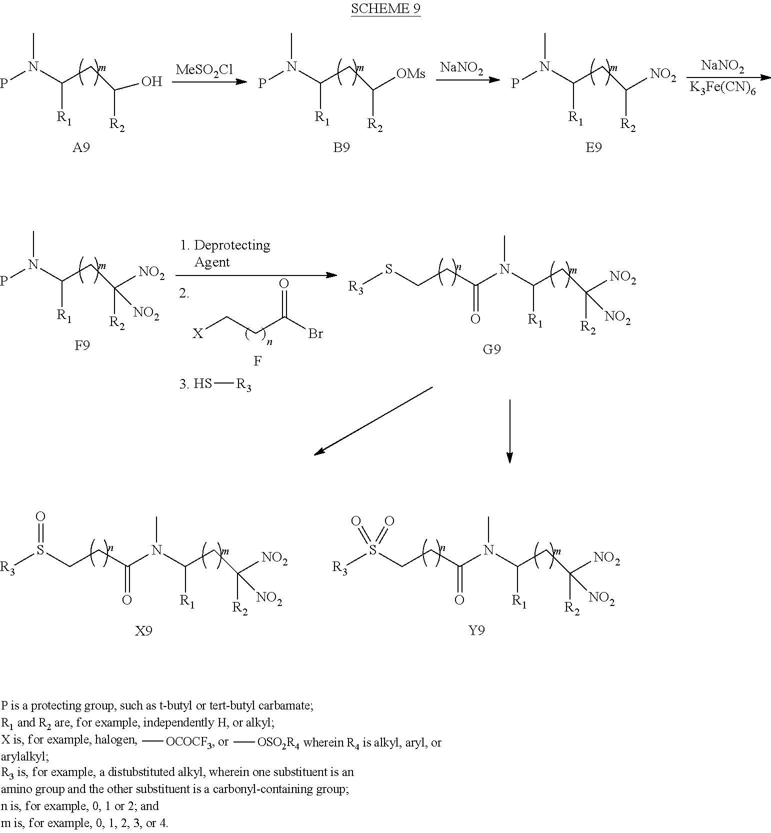

Scheme 10 illustrates an alternative procedure for preparing mono-nitro compounds. Reaction of dinitro compound A10 with thiol compound B10 provides mono-nitro compound C10. The reaction can be performed at room temperature, or the reaction mixture can be heated to achieve a temperature higher than room temperature. One or more equivalents of thiol B10 may be used, relative to the amount of dinitro compound A10. One exemplary thiol B10 that can be used in the procedure is cysteine. A more specific illustration of this synthetic procedure is the reaction of dinitro compound A10′ with cysteine (B10′) to provide mono-nitro compound C10′. Oxidation of thioether C10′ then provides sulfoxide X10 and sulfone Y10. Procedures for converting a thioether to a sulfoxide or sulfone using oxidizing conditions can be found in the literature.

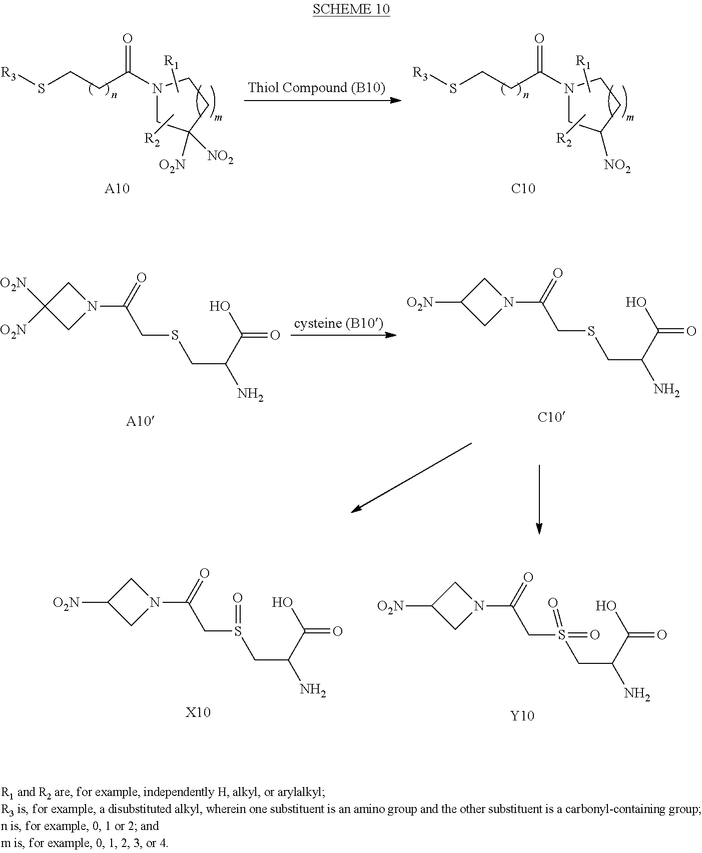

### IV. Therapeutic Applications

The invention provides methods of treating or preventing various medical disorders, such as an autoimmune disorder, inflammatory disorder, neurodegenerative disorder, or neuromuscular disorder using an organonitro or sulfoxyalkyl organonitro compound, e.g., RRx-001 and related compounds, and pharmaceutical compositions described herein. Treatment methods also include the use of an organonitro or sulfoxyalkyl organonitro or related compound described herein as part of a combination therapy with another therapeutic agent.

### Methods of Treating Autoimmune, Inflammatory, Neurodegenerative, and Neuromuscular Disorders

Another aspect of the invention provides a method of treating a disorder selected from the group consisting of an autoimmune disorder, inflammatory disorder, neurodegenerative disorder, or neuromuscular disorder. The method comprises administering to a patient in need thereof a therapeutically effective amount of an organonitro or sulfoxyalkyl organonitro or related compound described herein, such as RRx-001 or an analog thereof, which may be a compound of Formula II or III, to treat the disorder.

In certain embodiments, the disorder is an autoimmune disorder. In certain embodiments, the autoimmune disorder is selected from the group consisting of rheumatoid arthritis (RA), sarcoidosis, systemic lupus erythematosus (SLE), Huntington's disease, end stage renal disease, systemic sclerosis or scleroderma, myositis, diabetes type 1, multiple sclerosis. Sjögren's syndrome, psoriasis, primary biliary cirrhosis, autoimmune hepatitis, Graves' disease, Addison's disease, tuberculosis, Crohn's disease, ulcerative colitis, inflammatory bowel disease, autoimmune hemolytic anemia, idiopathic thrombocytopenic purpura, idiopathic neutropenia and coeliac disease.

In certain embodiments, the disorder is an inflammatory disorder. In certain embodiments, the inflammatory disorder is selected from the group consisting of sarcoidosis, vitiligo, polymyalgia rheumatica, graft vs. host disease (GvHD) or an autoimmune reaction after organ transplantation, Churg-Strauss syndrome, chronic gastritis, pernicious anemia, lichen sclerosis, long COVID, Huntington's disease, multiple sclerosis, asthma, Wegener's granulomatosis, autoimmune enteropathy, PANDAS, rheumatic fever, dermatomyositis, Goodpasture syndrome, primary biliary cirrhosis, diabetes type 1, autoimmune hepatitis, Graves' disease, Crohn's disease, ulcerative colitis, coeliac disease, Addison's disease, Sjogren's syndrome, systemic lupus erythematosus and rheumatoid arthritis.

In certain embodiments, the disorder is a neurodegenerative disorder. In certain embodiments, the neurodegenerative disorder is selected from the group consisting of Alzheimer's disease and other dementias, Parkinson's disease and PD-related disorders, multiple system atrophy, prion diseases, motor neuron diseases (MND), Huntington's disease, spinocerebellar ataxia, spinal muscular atrophy, Batten disease, Freidreich's ataxia, vascular dementia, transactive response DNA-binding protein-43, proteinopathies, incurable neurodegenerative diseases with pediatric onset, purine and pyrimidine defects, metal metabolism such as Wilson disease, pantothenate kinase associated neurodegeneration and neurodegeneration with brain iron accumulation, leukodystrophy, peroxisomal disorders, lysosomal storage disorders, congenital disorders of glycosylation, creatine disorders, and Rasmussen's encephalitis.

In certain embodiments, the disorder is a neuromuscular disorder. In certain embodiments, the neuromuscular disorder is selected from the group consisting of amyotrophic lateral sclerosis, multiple sclerosis, myasthenia gravis, Charcot-Marie-Tooth disease and related hereditary neuropathies, chronic inflammatory demyelination polyneuropathy, Guillain-Barre syndrome, Lambert Eaton syndrome. Miller-Fisher, muscular dystrophies, myopathies, peripheral neuropathies, neurotransmitter defects, immunoglobulin M gammopathy-associated neuropathy paraneoplastic neurological syndrome, and stiff man syndrome.

Another aspect of the disclosure provides a method for increasing compliance and tolerability in a subject in need of treatment for an autoimmune disorder, inflammatory disorder, neurodegenerative disorder, or neuromuscular disorder. In some embodiments, the method comprises administering a therapeutically effective amount of RRx-001 or an analog thereof. In some embodiments, administering the therapeutically effective amount of RRx-001 or analog thereof does not cause side effects selected from the group consisting of hematologic effects, neurologic effects, pulmonary effects, metabolic effects, cardiovascular effects, dermatologic effects, nephrologic effects, gastrointestinal effects, genitourinary effects, inflammatory effects, autoimmune effects, thyroidal effects, and immunodeficiency-related effects. In some embodiments, the subject completes treatment with a cumulative dose of at least 1 mg or 1 mg/m² of RRx-001 or an analog thereof.

In some aspect, provided herein are methods of treating a disorder or condition associated with interleukin 1 beta (IL-1β) or interleukin 18 (IL-18) expression or release. In some embodiments, the method comprises administering an effective amount of RRx-001 or an analog thereof to ameliorate a symptom of the disorder or condition. In some embodiments, the disorder or condition is an autoimmune disorder or an inflammatory disorder. In some embodiments, the disorder or condition is pediatric fever syndrome, acne vulgaris, hidradenitis suppurativa, psoriasis, rheumatoid, systemic juvenile idiopathic arthritis sepsis, chronic inflammation, aging, viral infection, asthma, congestive heart failure, angina, liver steatosis, diabetes, familial cold autoinflammatory syndrome (FCAS), Muckle-Wells syndrome (MWS), neonatal-onset multisystem inflammatory disease (NOMID), chronic infantile neurologic, cutaneous, arthritis (CINCA) syndrome, or cerebral edema due to intracerebral hemorrhage. In some embodiments, administering an effective amount of RRx-001 or an analog thereof does not lead to severe infection or end organ toxicity.

In some embodiments, the method further comprises administering a second agent. In some embodiments, the second agent comprises an IL-1α inhibitor, an IL-1β inhibitor, an IL-18 inhibitor, or an inflammasome inhibitor, or any combination thereof. In some embodiments, the second agent comprises canakinumab, anakinra, rilonacept, resveratrol, curcumin, MABp1, GSK-1070806 antibody, MCC950, β-hydroxybutyrate, glibenclamide, oridonin, JC121, JC124, YQ128, apigenin, cardamonin, Isoliquiritigenin, Glycyrrhizin, Luteolin, Quercetin, Artemisia, Caffeic acid phenethyl ester, Obovatol, Parthenolide, Shikonin, Sulforaphane, Tranilast, OLT1177 (Dapansutrile), CY-09, Methylene Blue, Disulfuram (Antabuse), Fenamic acid derivatives, Fluoxetine. β-Hydroxybutyrate (BHB), BAY 11-7082 (BAY), or X-11-5-27, or any combination thereof. In some embodiments, a side effect associated with the second agent is reduced.

In some aspect, provided herein are methods of controlling activity of a mononuclear phagocyte. In some embodiments, the method comprises exposing the mononuclear phagocyte to an effective amount of RRx-001 or an analog thereof. In some embodiments, the activity of the mononuclear phagocyte is expression or release of interleukin 1 beta (IL-1β) from the mononuclear phagocyte. In some embodiments, controlling activity of the mononuclear phagocyte is reducing the expression or release of interleukin 1 beta (IL-1β) from the mononuclear phagocyte. In some embodiments, the mononuclear phagocyte is a macrophage or a dendritic cell, or a combination thereof. In some embodiments, the exposure occurs ex vivo. In some embodiments, the mononuclear phagocyte is collected from a human subject. In some embodiments, the mononuclear phagocyte exposed to the effective amount of RRx-001 or an analog thereof is re-introduced into the human subject. In some embodiments, the mononuclear phagocyte exposed to the effective amount of RRx-001 or an analog thereof is re-introduced into the human subject by infusion. In some embodiments, immunological tolerance is increased in the human subject after the mononuclear phagocyte is re-introduced.

### Additional Features of the Therapeutic Methods

The therapeutic methods may be further characterized according to, for example, the identity of the patient to treated. In certain embodiments, the patient is a human.

The therapeutic methods may be further characterized according to, for example, the identity of the organonitro or sulfoxyalkyl organonitro or related compound used. For example, in certain embodiments, the compound is one of the generic or specific compounds described in Section III, such as, RRx-001, a compound of Formula II, a compound embraced by one of the further embodiments describing definitions for certain variables of Formula II, a compound of Formula III, a compound embraced by one of the further embodiments describing definitions for certain variables of Formula III, a compound of Formula II-A, or a compound embraced by one of the further embodiments describing definitions for certain variables of Formula II-A.

The description above describes multiple embodiments relating to methods of treating various disorders using certain organonitro or sulfoxyalkyl organonitro and related compounds. The patent application specifically contemplates all combinations of the embodiments. For example, the invention contemplates methods for autoimmune disorders, inflammatory disorders, neurodegenerative disorders, or neuromuscular disorders by administering a therapeutically effective amount of a compound of Formula II-A wherein A¹ is N, R¹ is hydrogen, R⁴ is —CH₂C(H)(X¹)X², and p is 1.

In certain embodiments, the compound is

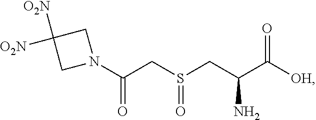 [SMILES: N[C@@H](CS(=O)CC(=O)N1CC([N+](=O)[O-])([N+](=O)[O-])C1)C(=O)O]

or a pharmaceutically acceptable salt thereof. In certain other embodiments, the compound is

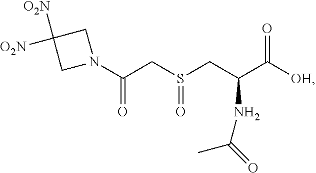 [SMILES: CC(=O)N[C@@H](CS(=O)CC(=O)N1CC([N+](=O)[O-])([N+](=O)[O-])C1)C(=O)O]

or a pharmaceutically acceptable salt thereof. In another embodiment, the compound is

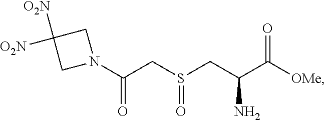 [SMILES: COC(=O)[C@@H](N)CS(=O)CC(=O)N1CC([N+](=O)[O-])([N+](=O)[O-])C1]

or a pharmaceutically acceptable salt thereof. In one embodiment, the compound is

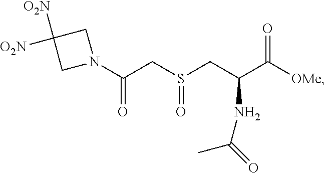 [SMILES: COC(=O)[C@H](CS(=O)CC(=O)N1CC([N+](=O)[O-])([N+](=O)[O-])C1)NC(C)=O]

or a pharmaceutically acceptable salt thereof. In certain other embodiments, the compound is

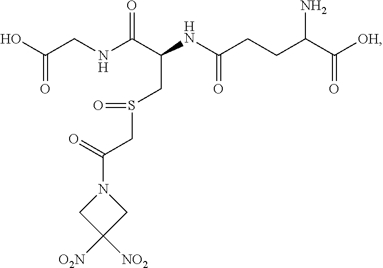 [SMILES: NC(CCC(=O)N[C@@H](CS(=O)CC(=O)N1CC([N+](=O)[O-])([N+](=O)[O-])C1)C(=O)NCC(=O)O)C(=O)O]

or a pharmaceutically acceptable salt thereof. In certain other embodiments, the compound is

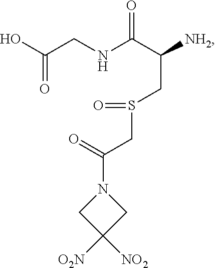 [SMILES: N[C@@H](CS(=O)CC(=O)N1CC([N+](=O)[O-])([N+](=O)[O-])C1)C(=O)NCC(=O)O]

or a pharmaceutically acceptable salt thereof. In certain embodiments, the compound is

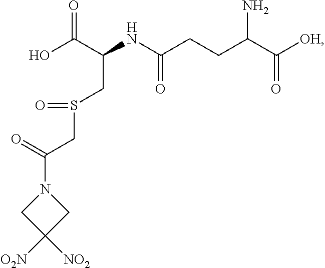 [SMILES: NC(CCC(=O)N[C@@H](CS(=O)CC(=O)N1CC([N+](=O)[O-])([N+](=O)[O-])C1)C(=O)O)C(=O)O]

or a pharmaceutically acceptable salt thereof.

In certain embodiments, the compound is

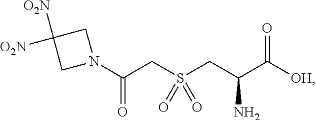 [SMILES: N[C@@H](CS(=O)(=O)CC(=O)N1CC([N+](=O)[O-])([N+](=O)[O-])C1)C(=O)O]

or a pharmaceutically acceptable salt thereof. In certain other embodiments, the compound is

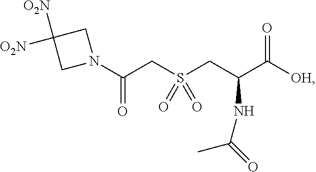 [SMILES: CC(=O)N[C@@H](CS(=O)(=O)CC(=O)N1CC([N+](=O)[O-])([N+](=O)[O-])C1)C(=O)O]

or a pharmaceutically acceptable salt thereof. In another embodiment, the compound is

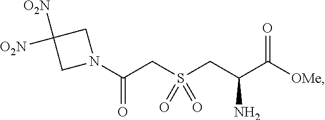 [SMILES: COC(=O)[C@@H](N)CS(=O)(=O)CC(=O)N1CC([N+](=O)[O-])([N+](=O)[O-])C1]

or a pharmaceutically acceptable salt thereof. In one embodiment, the compound is

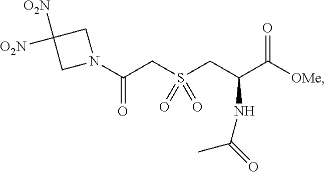 [SMILES: COC(=O)[C@H](CS(=O)(=O)CC(=O)N1CC([N+](=O)[O-])([N+](=O)[O-])C1)NC(C)=O]

or a pharmaceutically acceptable salt thereof. In certain other embodiments, the compound is

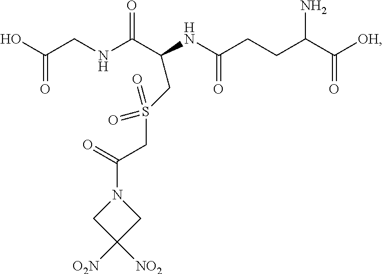 [SMILES: NC(CCC(=O)N[C@@H](CS(=O)(=O)CC(=O)N1CC([N+](=O)[O-])([N+](=O)[O-])C1)C(=O)NCC(=O)O)C(=O)O]

or a pharmaceutically acceptable salt thereof. In certain other embodiments, the compound is

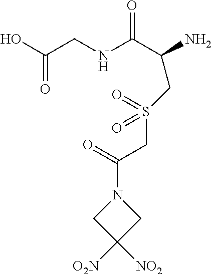 [SMILES: N[C@@H](CS(=O)(=O)CC(=O)N1CC([N+](=O)[O-])([N+](=O)[O-])C1)C(=O)NCC(=O)O]

or a pharmaceutically acceptable salt thereof.

In certain embodiments, the compound is

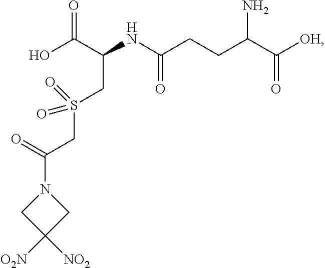 [SMILES: NC(CCC(=O)N[C@@H](CS(=O)(=O)CC(=O)N1CC([N+](=O)[O-])([N+](=O)[O-])C1)C(=O)O)C(=O)O]

or a pharmaceutically acceptable salt thereof.

### Combination Therapy

As indicated above, the invention embraces combination therapy, which includes the administration of an organonitro or sulfoxyalkyl organonitro or related compound described herein (such as RRx-001 or a compound of Formula II, III or II-A) and a second agent as part of a specific treatment regimen intended to provide the beneficial effect from the co-action of these therapeutic agents. The beneficial effect of the combination may include pharmacokinetic or pharmacodynamic co-action resulting from the combination of therapeutic agents.

Administration of these therapeutic agents in combination typically is carried out over a defined time period (e.g., hours or days depending upon the combination selected). The combination therapy may involve administration of two or more of these therapeutic agents as part of separate monotherapy regimens that result in the combinations of the present invention. Combination therapy also includes administration of these therapeutic agents in a sequential manner, that is, wherein each therapeutic agent is administered at a different time, as well as administration of these therapeutic agents, or at least two of the therapeutic agents, in a substantially simultaneous manner. Substantially simultaneous administration can be accomplished, for example, by administering to the subject a single capsule having a fixed ratio of each therapeutic agent or in multiple, single capsules for each of the therapeutic agents. Sequential or substantially simultaneous administration of each therapeutic agent can be affected by any appropriate route including, but not limited to, oral routes, intravenous routes, intramuscular routes, and direct absorption through mucous membrane tissues.

It is understood that the therapeutic agents can be administered by the same route or by different routes. For example, a first therapeutic agent of the combination selected may be administered by pulmonary administration while the other therapeutic agent(s) of the combination may be administered orally. Alternatively, for example, all therapeutic agents may be administered orally or all therapeutic agents may be administered by pulmonary administration. In certain embodiments, the second therapeutic agent is selected from the group consisting of a corticosteroid, immunosuppressants, and a disease-modifying anti-rheumatic drug (DMARD). In certain embodiments, the corticosteroid, immunosuppressant, or disease-modifying anti-rheumatic drug is selected from the group consisting of NSAIDS, including ibuprofen; glucocorticoid, including prednisone and dexamethasone; azathioprine; cyclosporin A; mycophenolate mofetil; tacrolimus; anti-T lymphocyte globulin, anti-CD3 antibodies, including muromonab; anti-CD25 antibodies, including basiliximab and daclizumab; anti-TNF-α antibodies, including infliximab and adalimumab; azathioprine; methotrexate; cyclosporin; sirolimus; everolimus; fingolimod; CellCept®; myfortic and cyclophosphamide. (Disease-modifying antirheumatic drugs (DMARDS), which through their different mechanisms of action interfere with critical pathways in the inflammatory cascade and thereby decrease inflammation, include: Sirolimus and everolimus, mTOR inhibitors that impede the translation of mRNA-encoding proteins which are necessary to the cell cycle, thus reducing T cell proliferation and cytokine production; azathioprine and mycophenolic acid that comes in two forms, mycophenolate mofetil (MMF) or CellCept and enteric-coated mycophenolate sodium or myfortic, which inhibit the purine pathway, necessary for DNA synthesis, tacrolimus, which inhibits the calcineurin pathway and lymphocyte activation, hydrocholoroquine, which inhibits intracellular toll-like receptor TLR9, leflunomide, which inhibits the mitochondrial enzyme dihydroorotate dehydrogenase (DHODH) that plays a key role in the de novo synthesis of uridine monophosphate (rUMP) required for the synthesis of DNA and RNA, methotrexate, which stimulates adenosine release from fibroblasts, reduces neutrophil adhesion, inhibits leukotriene B4 synthesis by neutrophils, inhibits local IL-1 production, reduces levels of IL-6 and IL-8, suppresses cell-mediated immunity, and inhibits synovial collagenase gene expression, sulfasalazine, which ameliorates oxidative, nitrative and nitrosative damage, minocycline, an antibiotic with anti-inflammatory properties, fingolimod, a sphingosine-1-phosphate (SIP) receptor modulator that prevents T and B lymphocyte egress from lymph nodes and significantly reduces peripheral blood lymphocyte counts and cyclophosphamide, an alkylating agent, which alkylates or binds to DNA and impairs its replication and transcription, leading to cell death. Biologics or more accurately, biologic DMARDS or bDMARDS, which are more targeted to specific pathways of the immune system include: Tumor necrosis factor (TNF) inhibitors: adalimumab (Humira), certolizumab pegol (Cimzia), etanercept (Enbrel), golimumab (Simponi), and infliximab (Remicade); Interleukin-1 inhibitor: anakinra (Kineret); Interleukin-6 inhibitors: tocilizumab (Actemra), sarilumab (Kevzara); T cell inhibitor: abatacept (Orencia); B cell inhibitor: rituximab (Rituxan); JAK inhibitors: tofacitinib (Xeljanz), baricitinib (Olumiant), upadacitinib (Rinvoq); anti-CD25 antibodies, including basiliximab and daclizumab, that bind to the human interleukin-2 receptor.

In some embodiments, the second therapeutic agent is a corticosteroid. In some embodiments, the corticosteroid is selected from the group consisting of cortisone, dexamethasone, hydrocortisone, ethamethasoneb, fludrocortisone, methylprednisolone, prednisone, prednisolone, and triamcinolone.

In some embodiments, the second therapeutic agent is an immunosuppressant. In some embodiments, the immunosuppressant is selected from the group consisting of NSAIDS, including aspirin, celecoxib, diclofenac, diclofenac/misoprostol, diflunisal, etodolac, ibuprofen, indomethacin, ketoprofen, nabumetone, naproxen, naproxen/esomeprazole, naproxen/lansoprazole, oxaprozin, piroxicam, salsalate, sulindac, and tometin; anti-T lymphocyte globulin, anti-Cd3 antibodies, including muromonab; anti-CD25 antibodies, including basiliximab and daclizumab; rituximab; sirolimus; everolimus; fingolimod; and myfortic.

In some embodiments, the second therapeutic agent is a disease-modifying anti-rheumatic drug (DMARD). In some embodiments, the DMARD is selected from the group consisting of azathioprine, auranofin, chloroquine, hydroxychloroquine, leuflonomide, minocycline, penicillamine, sulfasalazine, methotrexate, cyclosporin A, cyclophosphamide, tacrolimus, gold sodium thiomalate, tofacitinib, abatacept, adalimumab, anakinra, certolizumab, etanercept, golimumab, infliximab, rituximab, tocilizumab, and mycophenolate mofetil.

In some embodiments, the second therapeutic agent is an anti-inflammatory drug. In some embodiments, the anti-inflammatory drug is selected from aspirin, celecoxib, diclofenac, diclofenac/misoprostol, diflunisal, etodolac, ibuprofen, indomethacin, ketoprofen, nabumetone, naproxen, naproxen/esomeprazole, naproxen/lansoprazole, oxaprozin, piroxicam, salsalate, sulindac, and tometin.

In some embodiments, the second therapeutic agent is an anti-neurodegenerative agent. In some embodiments, the anti-neurodegenerative agent is selected from the group consisting of aducanumab, donepezil, tacrine, rivastigmine, galantamine, memantine, levodopa, carbidopa, benserazide, tolcapone, entacapone, selegiline, rasagiline, apomorphine, pramipexole, amantadine, trihexyphenidyl, benztropine, istradefyline, riluzole, edaravone, and tetrabenazine.

In some embodiments, RRx-001 or an analog thereof reduces the toxicity of a co-administered or subsequently administered NSAID, corticosteroid, DMARD, or biologic DMARD. In some embodiments, the toxicity is hematologic, neurologic, pulmonary, metabolic, cardiovascular, dermatologic, nephrologic, gastrointestinal, genitourinary, inflammatory, autoimmune-related, thyroidal, and/or bone marrow-related, or combinations thereof. In some embodiments, the subject receiving the combination therapy has an autoimmune disorder, inflammatory disorder, neuromuscular disorder, or neurodegenerative disorder.

### V. Pharmaceutical Compositions

The invention provides pharmaceutical compositions comprising a pharmaceutical carrier and an organonitro or sulfoxyalkyl organonitro or related compound described herein, such as RRx-001 or a compound of Formula II or III. In certain embodiments, the organonitro or sulfoxyalkyl organonitro or related compound is defined by one or more of the particular embodiments described above in Section III, such as where the organonitro or sulfoxyalkyl organonitro compound is RRx-001 or a compound Formula II, A¹ is N, R² and R³ are hydrogen, m is 0, and n is 2.

In certain embodiments, the pharmaceutical compositions preferably comprise a therapeutically effective amount of one or more of the organonitro or sulfoxyalkyl organonitro or related compounds described above, formulated together with one or more pharmaceutically acceptable carriers (additives) and/or diluents. As described in detail below, the pharmaceutical compositions of the present invention may be specially formulated for administration in solid or liquid form, including those adapted for the following: (1) oral administration, for example, drenches (aqueous or non-aqueous solutions or suspensions), tablets (e.g., those targeted for buccal, sublingual, and/or systemic absorption), boluses, powders, granules, pastes for application to the tongue; (2) parenteral administration by, for example, subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; or (8) nasally.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Formulations of the present invention include those suitable for oral, nasal, topical (including buccal and sublingual), rectal, vaginal and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient that can be combined with a carrier material to produce a single dosage form will generally be that amount of the compound which produces a therapeutic effect. Generally, out of one hundred percent, this amount will range from about 0.1 percent to about ninety-nine percent of active ingredient, preferably from about 5 percent to about 70 percent, most preferably from about 10 percent to about 30 percent.

In certain embodiments, a formulation of the present invention comprises an excipient selected from the group consisting of cyclodextrins, celluloses, liposomes, micelle forming agents, e.g., bile acids, and polymeric carriers, e.g., polyesters and polyanhydrides; and a compound of the present invention. In certain embodiments, an aforementioned formulation renders a compound of the present invention orally bioavailable.

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. A compound of the present invention may also be administered as a bolus, electuary or paste. In some embodiments, a compound of the present invention may be administered as a swish and spit and/or as a swish and swallow formulation.

In solid dosage forms of the invention for oral administration (capsules, tablets, pills, dragees, powders, granules, trouches and the like), the active ingredient is mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds and surfactants, such as poloxamer and sodium lauryl sulfate; (7) wetting agents, such as, for example, cetyl alcohol, glycerol monostearate, and non-ionic surfactants; (8) absorbents, such as kaolin and bentonite clay; (9) lubricants, such as talc, calcium stearate, magnesium stearate, solid polyethylene glycols, sodium lauryl sulfate, zinc stearate, sodium stearate, stearic acid, and mixtures thereof; (10) coloring agents; and (11) controlled release agents such as crospovidone or ethyl cellulose. In the case of capsules, tablets and pills, the pharmaceutical compositions may also comprise buffering agents. Solid compositions of a similar type may also be employed as fillers in soft and hard-shelled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be formulated for rapid release, e.g., freeze-dried. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration of the compounds of the invention include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions of the invention for rectal or vaginal administration may be presented as a suppository, which may be prepared by mixing one or more compounds of the invention with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Dosage forms for the topical or transdermal administration of a compound of this invention include powders, sprays, ointments, pastes, creams, lotions, gels, solutions, patches and inhalants. The active compound may be mixed under sterile conditions with a pharmaceutically-acceptable carrier, and with any preservatives, buffers, or propellants which may be required.

The ointments, pastes, creams and gels may contain, in addition to an active compound of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound of this invention, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the compound in the proper medium. Absorption enhancers can also be used to increase the flux of the compound across the skin. The rate of such flux can be controlled by either providing a rate controlling membrane or dispersing the compound in a polymer matrix or gel.

Ophthalmic formulations, eye ointments, powders, solutions and the like, are also contemplated as being within the scope of this invention.

Pharmaceutical compositions of this invention suitable for parenteral administration comprise one or more compounds of the invention in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain sugars, alcohols, antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms upon the subject compounds may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug administered by subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

Injectable depot forms are made by forming microencapsule matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

Organonitro or sulfoxyalkyl organonitro compounds and/or pharmaceutical compositions thereof may also be administered directly to the lung by inhalation. For administration by inhalation, organonitro or sulfoxyalkyl organonitro compounds and/or pharmaceutical compositions thereof may be conveniently delivered to the lung by a number of different devices. For example, a Metered Dose Inhaler (“MDI”), which utilizes canisters that contain a suitable low boiling propellant, (e.g., dichlorodifluoromethane, trichlorofluoromethane, dichlorotetrafluoroethane, carbon dioxide or any other suitable gas) may be used to deliver organonitro or sulfoxyalkyl organonitro compounds and/or pharmaceutical compositions thereof directly to the lung.

Alternatively, a Dry Powder Inhaler (“DPI”) device may be used to administer an organonitro or sulfoxyalkyl organonitro compound and/or pharmaceutical composition thereof to the lung. DPI devices typically use a mechanism such as a burst of gas to create a cloud of dry powder inside a container, which may then be inhaled by the patient, and are well known in the art. A popular variation is the multiple dose DPI (“MDDPI”) system, which allows for the delivery of more than one therapeutic dose. MDDPI devices are commercially available from a number of pharmaceutical companies (e.g., Schering Plough, Madison, NJ). For example, capsules and cartridges of gelatin for use in an inhaler or insufflator may be formulated containing a powder mix of an organonitro or sulfoxyalkyl organonitro compound and/or pharmaceutical composition thereof and a suitable powder base such as lactose or starch for these systems.

Another type of device that may be used to deliver a compound and/or pharmaceutical composition thereof to the lung is a liquid spray device supplied, for example, by Aradigm Corporation, Hayward, CA. Liquid spray systems use extremely small nozzle holes to aerosolize liquid drug formulations that may then be directly inhaled into the lung.

In some embodiments, a nebulizer is used to deliver an organonitro or sulfoxyalkyl organonitro compound and/or pharmaceutical composition thereof to the lung. Nebulizers create aerosols from liquid drug formulations by using, for example, ultrasonic energy to form fine particles that may be readily inhaled (see e.g., Verschoyle et al., British J. Cancer, 1999, 80, Suppl. 2, 96). Examples of nebulizers include devices supplied by Sheffield Pharmaceuticals, St. Louis, MO. (see, e.g., Armer et al., U.S. Pat. No. 5,954,047; van der Linden et al., U.S. Pat. No. 5,950,619; van der Linden et al., U.S. Pat. No. 5,970,974) and Batelle Pulmonary Therapeutics, Columbus. OH.

In other embodiments, an electrohydrodynamic (“EHD”) aerosol device is used to deliver an organonitro or sulfoxyalkyl organonitro compound and/or pharmaceutical composition thereof to the lung of a patient. EHD aerosol devices use electrical energy to aerosolize liquid drug solutions or suspensions (see e.g., Noakes et al., U.S. Pat. No. 4,765,539). The electrochemical properties of the formulation may be important parameters to optimize when delivering an organonitro or sulfoxyalkyl organonitro compound and/or pharmaceutical composition thereof to the lung with an EHD aerosol device and such optimization is routinely performed by one of skill in the art. EHD aerosol devices may more efficiently deliver drugs to the lung than existing pulmonary delivery technologies.

When the compounds of the present invention are administered as pharmaceuticals, to humans and animals, they can be given per se or as a pharmaceutical composition containing, for example, 0.1 to 99% (more preferably, 10 to 30%) of active ingredient in combination with a pharmaceutically acceptable carrier.

The preparations of the present invention may be given orally, parenterally, topically, or rectally. They are of course given in forms suitable for each administration route. For example, they are administered in tablets or capsule form, by injection, inhalation, eye lotion, ointment, suppository, etc. administration by injection, infusion or inhalation, topical by lotion or ointment; and rectal by suppositories. Oral administrations are preferred.

The phrase “parenteral administration” and “administered parenterally” as used herein means modes of administration other than enteral and topical administration, usually by injection, and include, without limitation, intravenous, intramuscular, intraarterial, intrathecal, intracapsular, intraorbital, intracardiac, intradermal, intraperitoneal, transtracheal, subcutaneous, subcuticular, intraarticulare, subcapsular, subarachnoid, intraspinal, and intrasternal injection and infusion.

The phrases “systemic administration,” “administered systemically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration.

These compounds may be administered to humans and other animals for therapy by any suitable route of administration, including orally, nasally, as by, for example, a spray, rectally, intravaginally, parenterally, intracistemally and topically, as by powders, ointments or drops, including buccally and sublingually.

Regardless of the route of administration selected, the compounds of the present invention, which may be used in a suitable hydrated form, and/or the pharmaceutical compositions of the present invention, are formulated into pharmaceutically-acceptable dosage forms by conventional methods known to those of skill in the art.

Actual dosage levels of the active ingredients in the pharmaceutical compositions of this invention may be varied so as to obtain an amount of the active ingredient that is effective to achieve the desired therapeutic response for a particular patient, composition, and mode of administration, without being toxic to the patient.

The selected dosage level will depend upon a variety of factors including the activity of the particular compound of the present invention employed, or the ester, salt or amide thereof, the route of administration, the time of administration, the rate of excretion or metabolism of the particular compound being employed, the rate and extent of absorption, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In general, a suitable daily dose of a compound of the invention will be that amount of the compound which is the lowest dose effective to produce a therapeutic effect. Such an effective dose will generally depend upon the factors described above. Preferably, the compounds are administered at about 0.01 mg/kg to about 200 mg/kg, more preferably at about 0.1 mg/kg to about 100 mg/kg, even more preferably at about 0.5 mg/kg to about 50 mg/kg. When the compounds described herein are co-administered with another agent (e.g., as sensitizing agents), the effective amount may be less than when the agent is used alone.

If desired, the effective daily dose of the active compound may be administered as two, three, four, five, six or more sub-doses administered separately at appropriate intervals throughout the day, optionally, in unit dosage forms. Preferred dosing is one administration per day.

Each dose of the formulation comprising RRx-001 or a compound of Formula II, II-A, II-B, or III can be administered to the subject by intravenous infusion. Depending on the circumstances, RRx-001 or the compound of Formula II, II-A, II-B, or III is pre-mixed with a sample of blood harvested from the subject blood prior to administration. Methods of performing blood transfusion by administering to a subject in need thereof a blood product including RRx-001 is described in WO2017/123593, the content of which is incorporated herein. A device for the intravenous infusion of compounds including RRx-001 is described in WO2019/241276, the content of which is incorporated herein.

### VI. Kits for Use in Medical Applications

Another aspect of the invention provides a kit for treating a disorder. The kit comprises: i) instructions for treating a disorder, such as an autoimmune disorder, inflammatory disorder, neurodegenerative disorder, or neuromuscular disorder; and ii) an organonitro or sulfoxyalkyl organonitro or related compound described herein, such as RRx-001 or a compound of Formula II or III. The kit may comprise one or more unit dosage forms containing an amount of an organonitro or sulfoxyalkyl organonitro or related compound described herein, such as RRx-001 or a compound of Formula II or Ill, that is effective for treating the disorder.

The description above describes multiple aspects and embodiments of the invention, including an organonitro or sulfoxyalkyl organonitro and related compounds, compositions comprising an organonitro or sulfoxyalkyl organonitro and related compounds, methods of using the organonitro or sulfoxyalkyl organonitro and related compounds and compositions comprising same, and kits. The patent application specifically contemplates all combinations and permutations of the aspects and embodiments. For example, the invention contemplates treating an autoimmune disorder, inflammatory disorder, neurodegenerative disorder, or neuromuscular disorder in a human patient by administering a therapeutically effective amount of a compound of Formula II-A. Further, for example, the invention contemplates a kit for treating an autoimmune disorder, inflammatory disorder, neurodegenerative disorder, or neuromuscular disorder, the kit comprising instructions for treating an autoimmune disorder, inflammatory disorder, neurodegenerative disorder, or neuromuscular disorder and ii) an organonitro or sulfoxyalkyl organonitro or related compound described herein, such as a compound of Formula II-A.

### ENUMERATED EMBODIMENTS

The following enumerated embodiments are representative of some aspects of the invention.

1. A method of treating a disorder selected from the group consisting of an autoimmune disorder, inflammatory disorder, neurodegenerative disorder, and neuromuscular disorder in a subject in need thereof, the method comprising administering a loading dose of RRx-001 or an analog thereof to the subject in an amount effective to ameliorate a symptom of the disorder, and thereafter administering a maintenance dose of RRx-001 or the analog thereof to maintain the amelioration of the symptom for a prolonged period of time.
2. The method of embodiment 1, wherein the loading dose comprises at least 1 mg (e.g., 1.5 mg, 2 mg, 2.5 mg or 3 mg) on a first day.
3. The method of embodiment 1, wherein the loading dose comprises at least 1 mg/m² (e.g., 1.5 mg/m², 2 mg/m², 2.5 mg/m², or 3 mg/m²) on a first day.
4. The method of embodiment 2 or 3, wherein the loading dose comprises multiple doses administered after the first day with a periodic interval of at least 3, 4, 5, 6, 7, 14, 21, or 28 days separating each dose so as to achieve amelioration of the symptom on the disorder.
5. The method of embodiment 4, wherein the doses administered after the first day are the same as the dose administered on the first day.
6. The method of any one of embodiments 1-5, wherein the maintenance dose is administered after the loading dose with a periodic interval of about 7 days, about 14 days, about 21 days, or about 28 days separating each dose so as to maintain amelioration of the symptom of the disorder.
7. The method of any one of embodiments 1-5, wherein the maintenance dose is administered after the loading dose with a periodic interval of 7 days, 14 days, 21 days, or 28 days separating each dose so as to maintain amelioration of the symptom of the disorder.
8. The method of any one of embodiments 1-7, wherein the maintenance dose is less than the loading dose.
9. The method of any one of embodiments 1-8, wherein the prolonged period of time is at least 1 month, or 3, 6, 9, or 12 months.
10. The method of any one of embodiments 1-9, wherein administering the loading dose and administering the maintenance dose does not lead to toxic systemic side effects.
11. The method of embodiment 10, wherein the toxic systemic side effects are selected from the group consisting of dyspnea, cough, fatigue, cardiac symptoms, electrolyte deficiencies, thyroid dysfunction, bone loss, sleep issues, nephrotoxicity, neurologic symptoms, autoimmunity, retinitis, hepatotoxicity, gastrointestinal distress, weight loss, malaise, rash, and leukopenia with increased risk of infection and the development of malignancy, and combinations thereof.
12. A method for increasing compliance and tolerability in a subject in need of treatment for an autoimmune disorder, inflammatory disorder, neurodegenerative disorder, or neuromuscular disorder, the method comprising administering a therapeutically effective amount of RRx-001 or an analog thereof; wherein administration of the therapeutically effective amount does not cause hematologic, neurologic, pulmonary, metabolic, cardiovascular, dermatologic, nephrologic, gastrointestinal, genitourinary, inflammatory, autoimmune, thyroidal, and immunodeficiency-related side effects; and wherein the subject completes treatment with a cumulative dose of at least 1 mg or 1 mg/m² of RRx-001 or an analog thereof.
13. A method of preventing the initiation, development or worsening of a symptom of a disorder selected from the group consisting of an autoimmune disorder, inflammatory disorder, neurodegenerative disorder, and neuromuscular disorder in a subject in need thereof, the method comprising administering an effective amount of RRx-001 or an analog thereof to the subject to prevent the initiation, development or worsening of the symptom of the disorder.
14. The method of embodiment 13, wherein the effective amount comprises at least 1 mg (e.g., 1.5 mg, 2 mg, 2.5 mg or 3 mg).
15. The method of embodiment 13, wherein the effective amount comprises at least 1 mg/m² (e.g., 1.5 mg/m², 2 mg/m², 2.5 mg/m², or 3 mg/m²).
16. The method of embodiment 14 or 15, wherein the RRx-001 or the analog thereof is administered to the subject periodically (e.g., once every week, two weeks, or three weeks, or 4 weeks, or once a month, two months, three months or four months).
17. The method of any one of embodiments 1-16, wherein the disorder is an autoimmune disorder.
18. The method of any one of embodiments 1-16, wherein the disorder is a metabolic disorder.
19. The method of any one of embodiments 1-16, wherein the disorder is a bone or joint disorder.
20. The method of embodiment 17, wherein the autoimmune disorder is selected from the group consisting of rheumatoid arthritis (RA), sarcoidosis, systemic lupus erythematosus (SLE), Huntington's disease, end stage renal disease, systemic sclerosis or scleroderma, myositis, diabetes type 1, multiple sclerosis, Sjögren's syndrome, psoriasis, primary biliary cirrhosis, autoimmune hepatitis, Graves' disease, Addison's disease, tuberculosis, Crohn's disease, ulcerative colitis, inflammatory bowel disease, autoimmune hemolytic anemia, idiopathic thrombocytopenic purpura, idiopathic neutropenia and coeliac disease.
21. The method of any one of embodiments 1-16, wherein the disorder is an inflammatory disorder.
22. The method of embodiment 21, wherein the inflammatory disorder is selected from the group consisting of sarcoidosis, vitiligo, polymyalgia rheumatica, graft vs. host disease (GvHD) or an autoimmune reaction after organ transplantation, Churg-Strauss syndrome, chronic gastritis, pernicious anemia, lichen sclerosis, long COVID, Huntington's disease, multiple sclerosis, asthma, Wegener's granulomatosis, autoimmune enteropathy, PANDAS, rheumatic fever, dermatomyositis, Goodpasture syndrome, primary biliary cirrhosis, diabetes type 1, autoimmune hepatitis, Graves' disease, Crohn's disease, ulcerative colitis, coeliac disease, Addison's disease, Sjogren's syndrome, systemic lupus erythematosus and rheumatoid arthritis.
23. The method of embodiment 18, wherein the metabolic disorder is selected from the group consisting of gout, type 2 diabetes, atherosclerosis, fatty liver disease, stroke and cardiovascular disease.
24. The method of embodiment 19, wherein the bone and joint disorder is selected from the group consisting of osteomyelitis, infectious arthritis, sarcoidosis, rheumatoid arthritis, ankylosing spondylitis, psoriatic arthritis, Lyme arthritis and osteoarthritis.
25. The method of any one of embodiments 1-16, wherein the disorder is a neurodegenerative disorder.
26. The method of embodiment 25, wherein the neurodegenerative disorder is selected from the group consisting of Alzheimer's disease and other dementias, Parkinson's disease (PD) and PD-related disorders, multiple system atrophy, prion diseases, motor neuron diseases (MND), Huntington's disease, spinocerebellar ataxia, spinal muscular atrophy, Batten disease, Freidreich's ataxia, vascular dementia, transactive response DNA-binding protein-43, proteinopathies, incurable neurodegenerative diseases with pediatric onset, purine and pyrimidine defects, metal metabolism such as Wilson disease, pantothenate kinase associated neurodegeneration and neurodegeneration with brain iron accumulation, leukodystrophy, peroxisomal disorders, lysosomal storage disorders, congenital disorders of glycosylation, creatine disorders, and Rasmussen's encephalitis.
27. The method of any one of embodiments 1-16, wherein the disorder is a neuromuscular disorder.
28. The method of embodiment 27, wherein the neuromuscular disorder is selected from the group consisting of' amyotrophic lateral sclerosis, multiple sclerosis, myasthenia gravis, Charcot-Marie-Tooth disease and related hereditary neuropathies, chronic inflammatory demyelination polyneuropathy, Guillain-Barre syndrome, Lambert Eaton syndrome, Miller-Fisher, muscular dystrophies, myopathies, peripheral neuropathies, neurotransmitter defects, immunoglobulin M gammopathy-associated neuropathy paraneoplastic neurological syndrome, and stiff man syndrome.
29. A method of treating an autoimmune disorder selected from the group consisting of rheumatoid arthritis (RA), sarcoidosis, systemic lupus erythematosus (SLE), Huntington's disease, end stage renal disease, systemic sclerosis or scleroderma, myositis, diabetes type 1, multiple sclerosis, Sjögren's syndrome, psoriasis, primary biliary cirrhosis, autoimmune hepatitis, Graves' disease, Addison's disease, tuberculosis, Crohn's disease, ulcerative colitis, inflammatory bowel disease, autoimmune hemolytic anemia, idiopathic thrombocytopenic purpura, idiopathic neutropenia and coeliac disease in a subject in need thereof, the method comprising administering an effective amount of RRx-001 or an analog thereof to ameliorate a symptom of the disorder.
30. A method of treating an inflammatory disorder selected from the group consisting of sarcoidosis, vitiligo, polymyalgia rheumatica, graft vs. host disease (GvHD) or an autoimmune reaction after organ transplantation, Churg-Strauss syndrome, chronic gastritis, pernicious anemia, lichen sclerosis, long COVID, Huntington's disease, multiple sclerosis, asthma, Wegener's granulomatosis, autoimmune enteropathy, PANDAS, rheumatic fever, dermatomyositis, Goodpasture syndrome, primary biliary cirrhosis, diabetes type 1, autoimmune hepatitis, Graves' disease, Crohn's disease, ulcerative colitis, coeliac disease, Addison's disease, Sjögren's syndrome, systemic lupus erythematosus and rheumatoid arthritis in a subject in need thereof, the method comprising administering an effective amount of RRx-001 or an analog thereof to ameliorate a symptom of the disorder.
31. A method of treating a neurodegenerative disorder selected from the group consisting of Alzheimer's disease and other dementias, Parkinson's disease and PD-related disorders, prion diseases, motor neuron diseases (MND), Huntington's disease, spinocerebellar ataxia, spinal muscular atrophy, Batten disease, Freidreich's ataxia, vascular dementia, multiple system atrophy, transactive response DNA-binding protein-43, proteinopathies, incurable neurodegenerative diseases with pediatric onset, purine and pyrimidine defects, metal metabolism such as Wilson disease, pantothenate kinase associated neurodegeneration and neurodegeneration with brain iron accumulation, leukodystrophy, peroxisomal disorders, lysosomal storage disorders, congenital disorders of glycosylation, creatine disorders, and Rasmussen's encephalitis in a subject in need thereof, the method comprising administering an effective amount of RRx-001 or an analog thereof to ameliorate a symptom of the disorder.
32. A method of treating Alzheimer's disease, the method comprising administering an effective amount of RRx-001 or an analog thereof to ameliorate a symptom of Alzheimer's disease.
33. A method of treating Parkinson's disease, the method comprising administering an effective amount of RRx-001 or an analog thereof to ameliorate a symptom of Parkinson's disease.
34. A method of treating a neuromuscular disorder selected from the group consisting of amyotrophic lateral sclerosis, multiple sclerosis, myasthenia gravis, Charcot-Marie-Tooth disease and related hereditary neuropathies, chronic inflammatory demyelination polyneuropathy, Guillain-Barre syndrome, Lambert Eaton syndrome, Miller-Fisher, muscular dystrophies, myopathies, peripheral neuropathies, neurotransmitter defects, immunoglobulin M gammopathy-associated neuropathy paraneoplastic neurological syndrome, and stiff man syndrome in a subject in need thereof, the method comprising administering an effective amount of RRx-001 or an analog thereof to ameliorate a symptom of the disorder.
35. The method of any one of embodiments 29-34, wherein the method comprises the dosing regimen of any one of embodiments 1-9.
36. The method of any one of embodiments 1-35, wherein the RRx-001 or analog thereof is combined with blood prior to administration to the subject.
37. The method of embodiment 36, wherein the blood is harvested from the subject.
38. The method of any one of embodiments 1-37, further comprising administering a second, different agent to the subject.
39. The method of any one of embodiments 1-38, wherein the subject is a human.
40. The method of any one of embodiments 1-39, wherein the RRx-001 or analog thereof reduces the toxicity of a co-administered or subsequently administered NSAID, corticosteroid, DMARD or biologic DMARD; wherein the toxicity is hematologic, neurologic, pulmonary, metabolic, cardiovascular, dermatologic, nephrologic, gastrointestinal-, genitourinary-, inflammatory, autoimmune-related, thyroidal and/or bone marrow-related; and wherein the subject has an autoimmune, inflammatory, neuromuscular or neurodegenerative disorder.
41. The method of any one of embodiments 1-40, wherein the RRx-001 or analog thereof is administered orally.
42. The method of any of embodiments 1-41, wherein the RRx-001 or analog thereof is administered in a swish and spit and/or swish and swallow formulation.
43. The method of any of embodiments 1-40, wherein the RRx-001 or analog thereof is administered sublingually.
44. The method of any of embodiments 1-40, wherein the RRx-001 or analog thereof is administered subcutaneously.
45. A pharmaceutical composition comprising RRx-001 or an analog thereof and at least one additional active component selected from the group consisting of an anti-inflammatory agent, a neuroprotective agent, a corticosteroid, an immunosuppressant, and a disease-modifying anti-rheumatic drug (DMARD), and one or more pharmaceutically acceptable excipients.

### EXAMPLES

The invention now being generally described, will be more readily understood by reference to the following examples, which are included merely for purposes of illustration of certain aspects and embodiments of the present invention, and are not intended to limit the invention.

### Example 1: Preclinical Study Evaluating the Effects of Sub-Chronic Administration of RRX-001 in an Alzheimer's Disease Mouse Model

The neuroprotective effects of RRx-001 administration on Alzheimer's Disease (AD), which has been linked to aberrant NLR family pyrin domain containing 3 (NLRP3) activation, were evaluated via intraperitoneal injection once weekly for 3 months on aged (18-21 months old) homozygous triple transgenic AD mouse model (3×Tg-AD, Jackson Labs). 3×Tg mice received either 2 mg/kg RRx-001 (n=3) or vehicle (n=3), and were evaluated against control non-transgenic mice (NonTg, Jackson Labs) that were untreated (n=3).

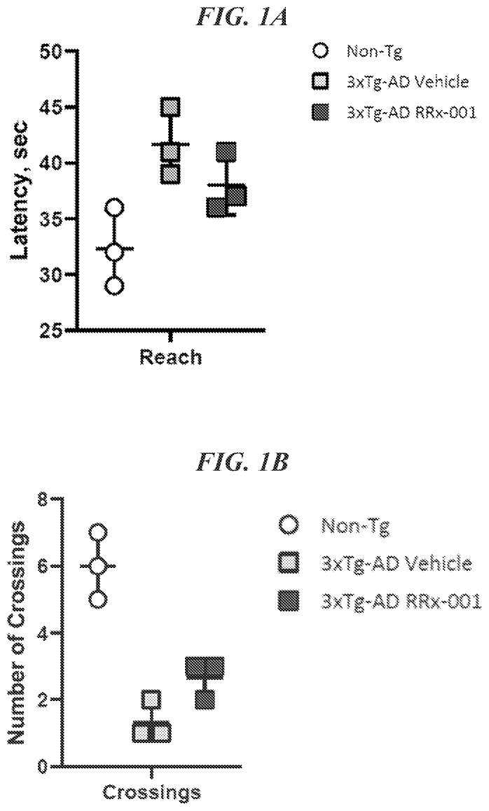

Five days after the last treatment, the mice were tested using a Morris water maze test. The next week, the mice were exposed to an elevated maze test. The latency to reach the exact hidden platform location on the Morris water maze test (FIG. 1A) and the number of crossings of the platform target quadrant (FIG. 1B) were recorded. Administration of RRx-001 was found to significantly reduce latency to locate the platform on the Morris water maze test, and also was associated with improvement in spatial learning tasks (FIG. 1C).

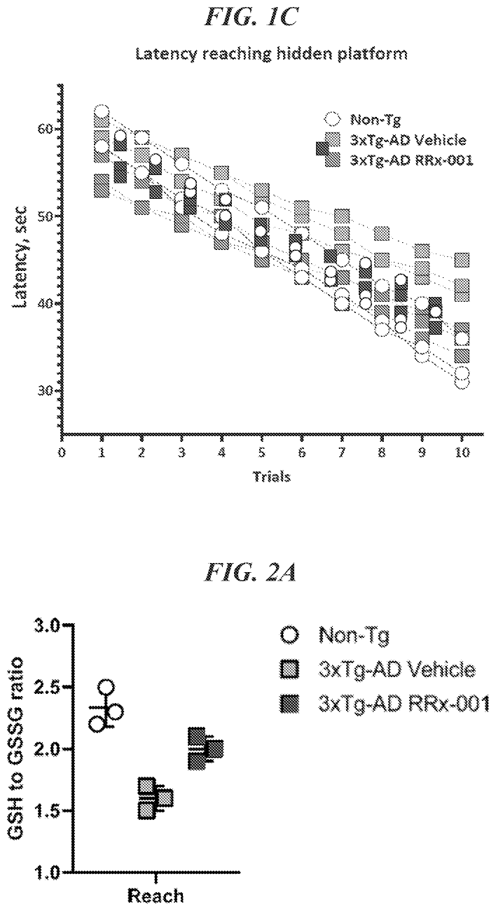

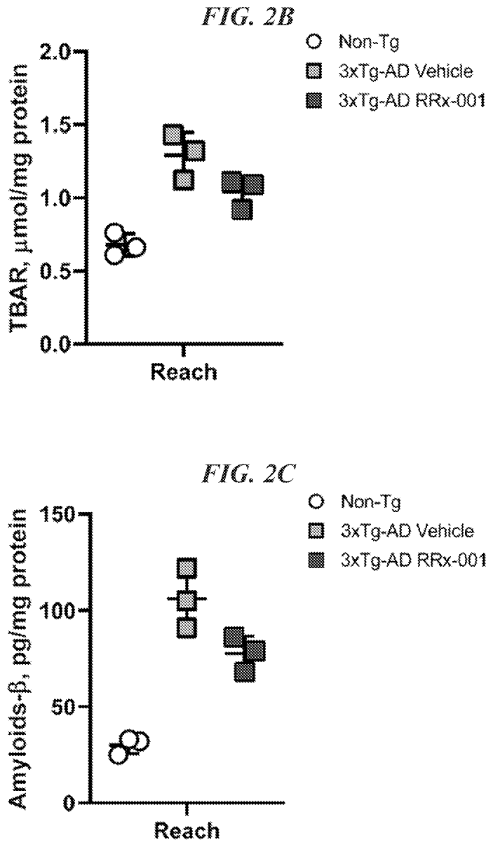

Further, a significant increase in reduced glutathione (GSH) compared to glutathione disulfide (GSSG) (FIG. 2A) and a decrease in lipid peroxidation (as measured by thiobarbituric acid-reactive substance (TBAR) levels) (FIG. 2B) and amyloid plaque density (FIG. 2C) were also observed in the 3×Tg-AD mice treated with RRx-001. Together, these findings suggest that RRx-001 reverses histological hallmarks of AD and protects cognitive and emotional function in aged 3×Tg-AD mice.

### Example 2: Effects of RRX-001 on Lipopolysaccharide (LPS) Challenged Mice

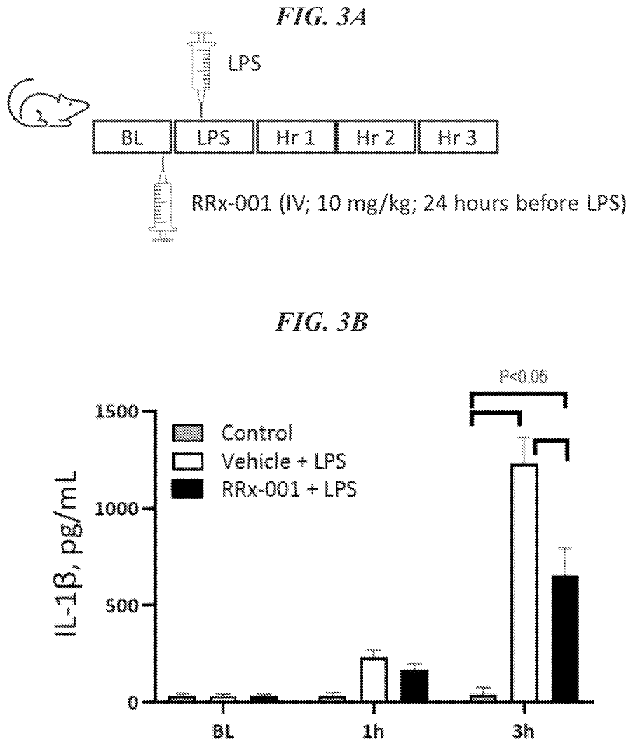

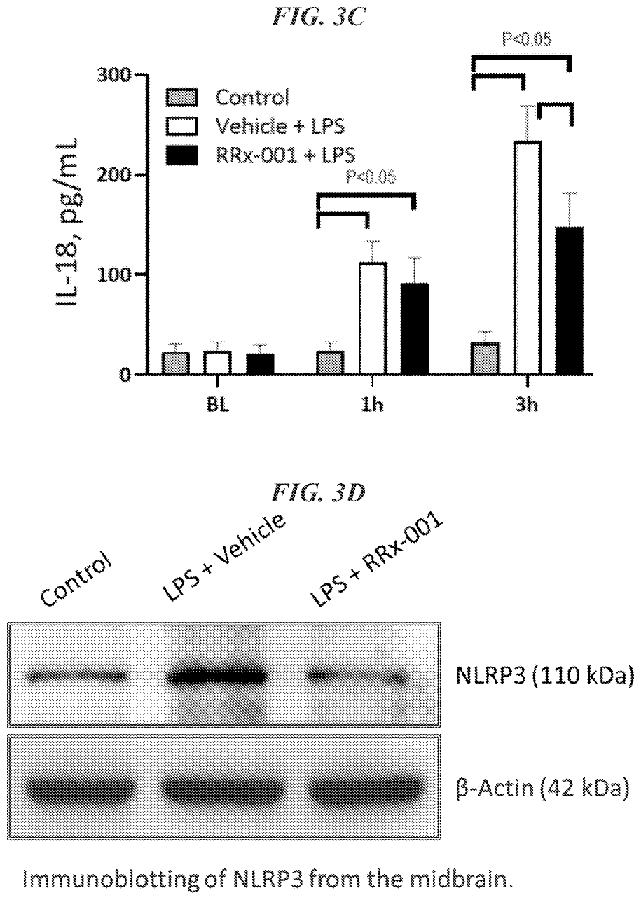

The protective effects of RRx-001 administration on Parkinson's Disease, which has been linked to aberrant NLR family pyrin domain containing 3 (NLRP3) activation, was evaluated by determining reductions in the levels of NLRP3 and serum concentration of interleukins 10 (IL-1β) and 18 (IL-18) in a mouse study. Mice were IV injected with vehicle or RRx-001 (10 mg/kg), 24 hours before lipopolysaccharide (LPS) challenge (20 mg/kg) (FIG. 3A). FIGS. 3B and 3C each depicts serum IL-1β or serum IL-18 levels measured at baseline, 1 hour after LPS injection, and 3 hours after LPS injection of mice in three groups: (i) control, (ii) LPS+vehicle, and (iii) LPS+RRx-001. FIG. 3D depicts immunoblotting of NLRP3 and β-actin from the midbrain of mice (euthanized 24 hours after LPS injection) in the three groups.

### Example 3: Effects of RRX-001 on LPS-Primed Bone Marrow Derived Macrophages (BMDM)

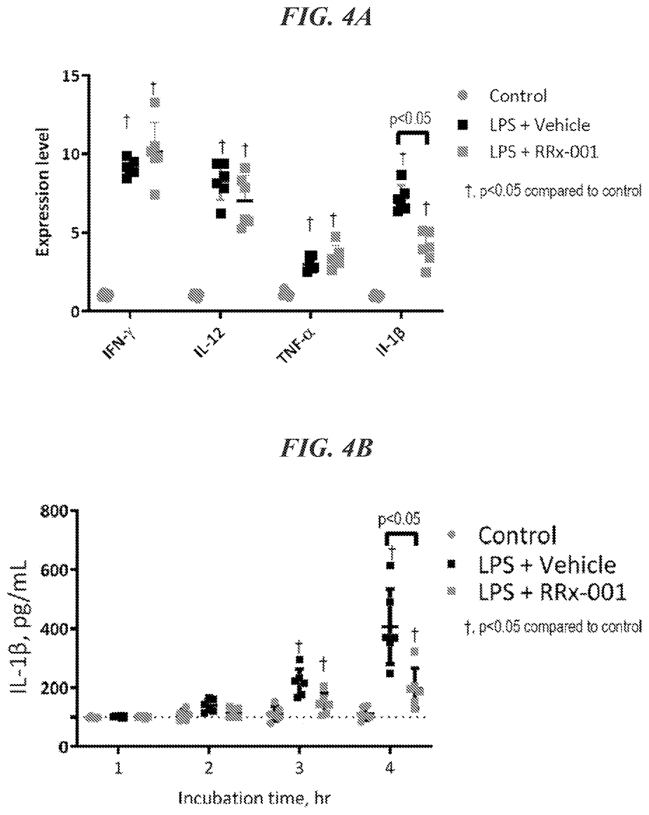

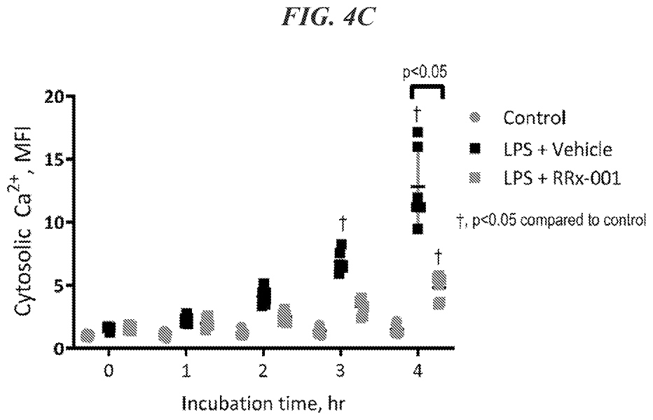

Bone marrow derived macrophages (BMDMs) were primed with LPS (50 ng/mL) for 30 minutes before being treated with RRx-001 (0.5 μM) for 4 hours. FIG. 4A depicts the expression levels of IFN-γ, IL-12, TNF-α, and IL-1β from the three groups: (i) control (not primed with LPS), (ii) LPS+vehicle (primed with LPS but not treated with RRx-001), (iii) and LPS+RRx-001 (primed with LPS and treated with RRx-001). FIG. 4B depicts IL-1β release over time from the three groups. FIG. 4C depicts cytosolic Ca²⁺ levels over time for the three groups. The results suggest that RRx-001 significantly reduces inflammation-driven expression of certain cytokines such as IL-1β, and prevents calcium accumulation in activated macrophages.

### Example 4: Effects of RRX-001 on Dextran Sulfate Sodium (DSS) Primed Bone Marrow Derived Macrophages (BMDMs)

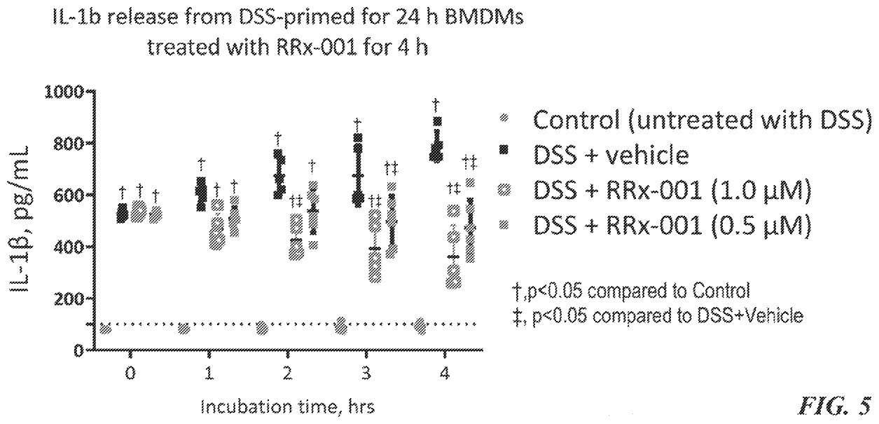

FIG. 5 depicts IL-1β release from the four groups of bone marrow derived macrophages (BMDMs): (i) control (not primed with DSS). (ii) DSS+vehicle (primed with DS but not treated with RRx-001), (iii) DSS+RRx-001 (0.5 μM; primed with DSS and treated with 0.5 μM RRx-001), and (iv) DSS+RRx-001 (1.0 μM; primed with DSS and treated with 1.0 μM RRx-001). The results suggest that RRx-001 significantly reduces the inflammatory effect of DSS.

### Example 5: Dextran Sulfate Sodium (DSS) Induced Colitis in Mice and Protective Effect of RRX-001

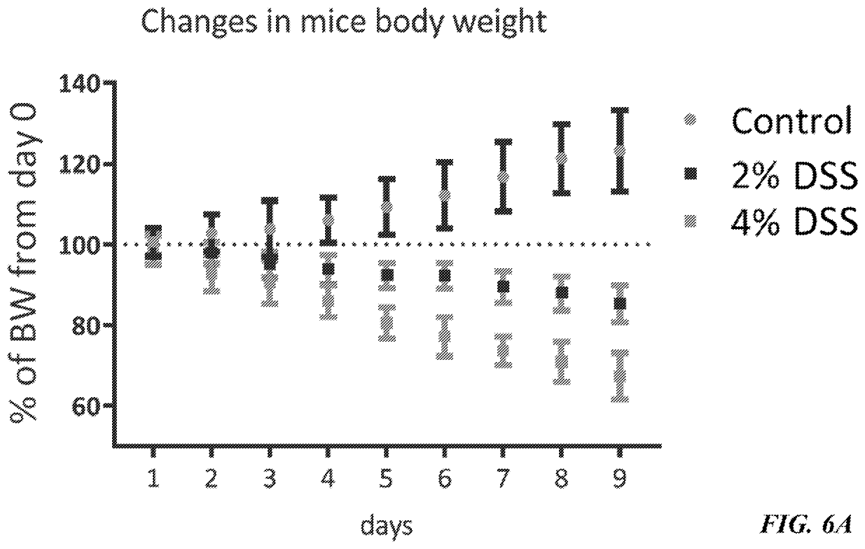

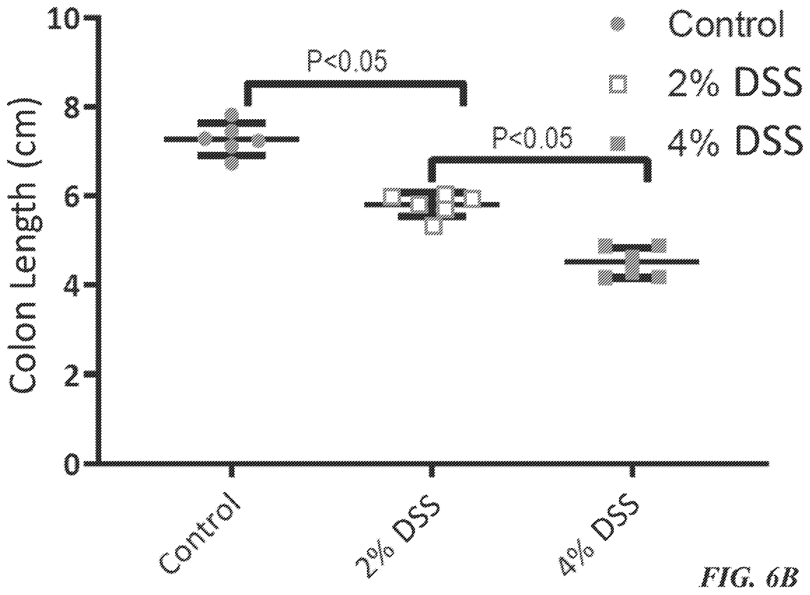

Colitis was induced in C57BL/6 mice with 2% or 4% dextran sulfate sodium (DSS; 40 kDa) dissolved in drinking water given ad libitum for 9 days. Control mice were given filtered water. FIG. 6A depicts changes in mice body weight over a 9-day period for each of the three groups: (i) control (filtered water), (ii) 2% DSS, and (iii) 4% DSS. FIG. 6B depicts colon length of mice in each group after the 9-day period.

Intravascular Injection (Mixed with Blood)

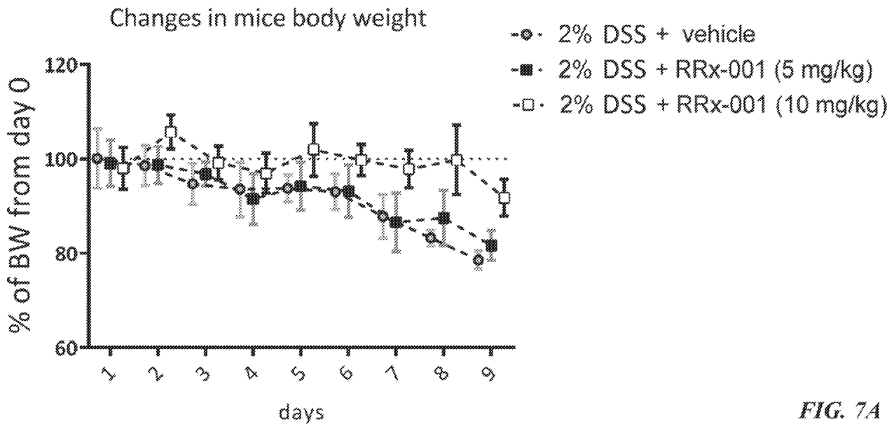

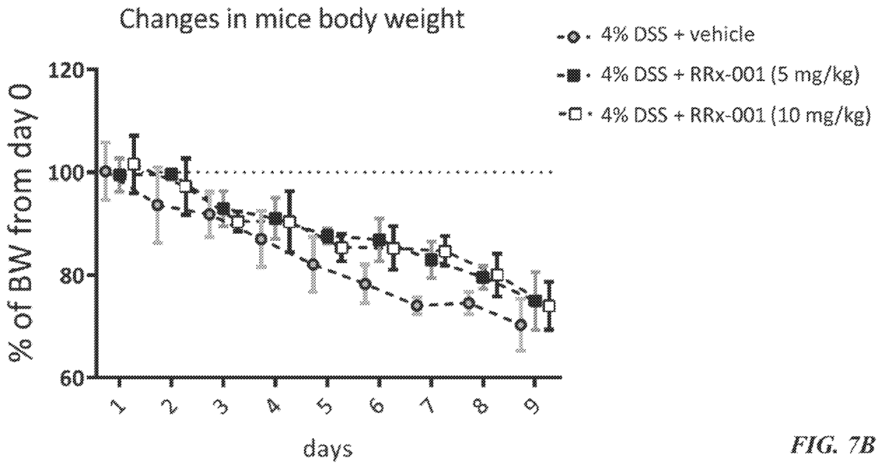

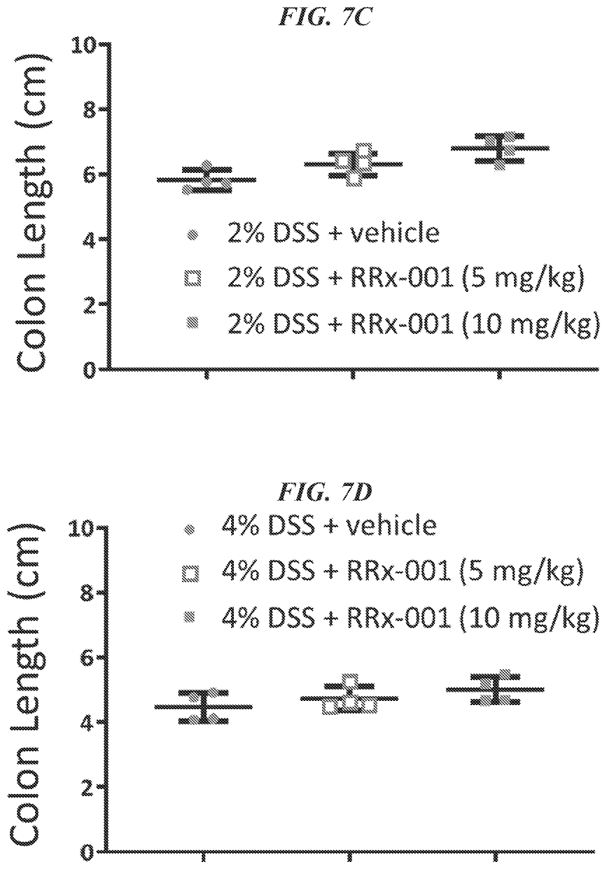

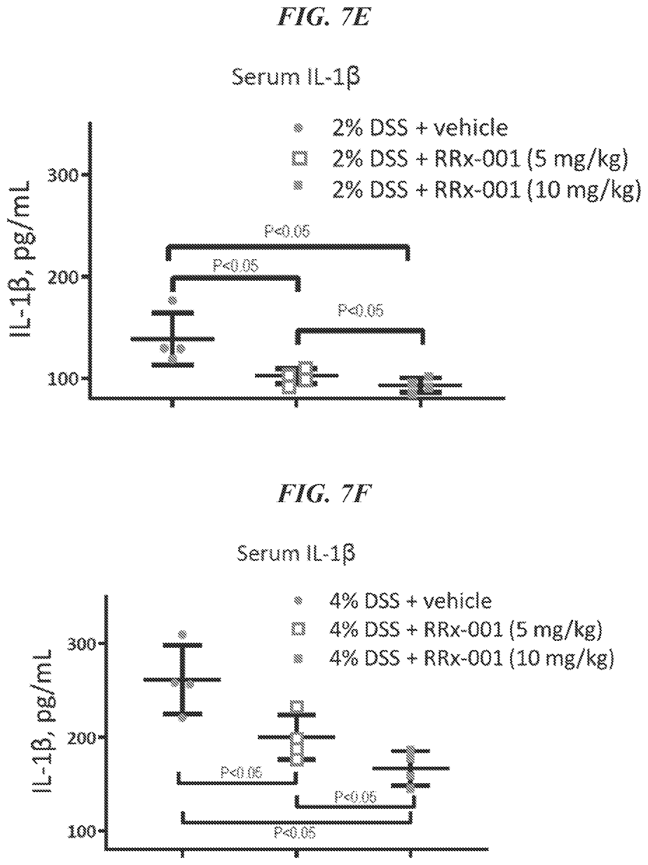

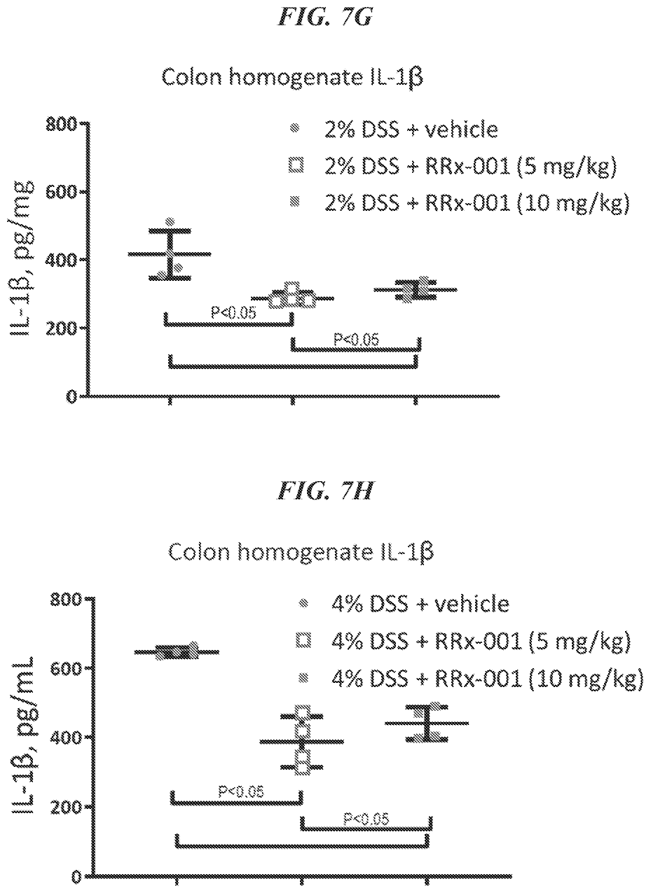

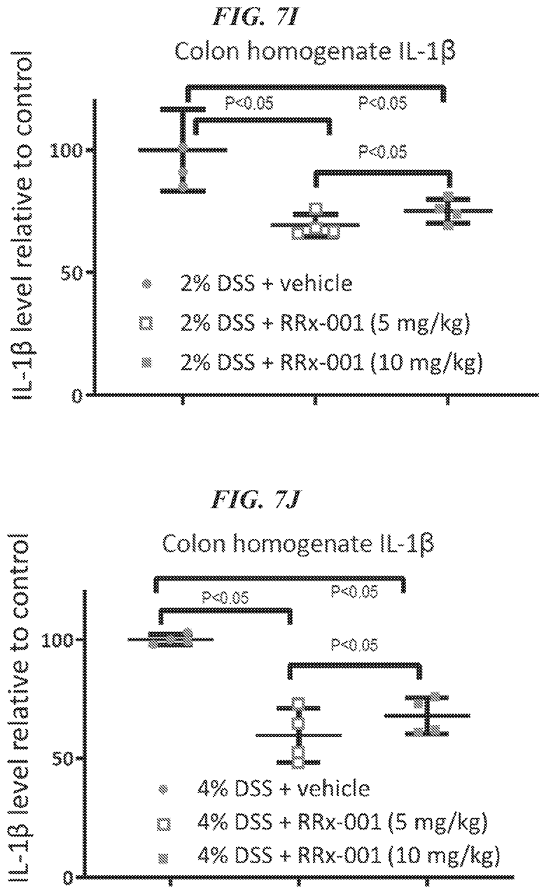

FIGS. 7A and 7B depict changes in mice body weight over a 9-day period for each of the six groups: (i) 2% DSS+vehicle, (ii) 2% DSS+RRx-001 (5 mg/kg), and (iii) 2% DSS+RRx-001 (10 mg/kg) in FIG. 7A; and (iv) 4% DSS+vehicle, (v) 4% DSS+RRx-001 (5 mg/kg), and (vi) 4% DSS+RRx-001 (10 mg/kg) in FIG. 7B. While the 2% or 4% DSS was given ad libitum for 9 days as described above, vehicle, 5 mg/kg RRx-001, or 10 mg/kg RRx-001 was given as intravascular injections of pre-mixed blood formulations every other day during the 9-day period. The results suggest that RRx-001 treatment significantly mitigates body weight loss in mice due to DSS induced colitis. FIGS. 7C and 7D depict colon length of mice in each of the six groups. The results suggest that RRx-001 treatment mitigates the colon length reduction due to DSS induced colitis. FIGS. 7E and 7F depict serum IL-1β levels in mice in each of the six groups. The results suggest that RRx-001 treatment reduces DSS-induced expression of serum IL-1β. FIGS. 7G and 7H depict colon homogenate IL-1β levels in mice in each of the six groups. FIGS. 7I and 7J depict colon homogenate IL-1β levels relative to the average IL-1β level of the control (i.e., DSS+vehicle) in mice in each of the six groups. The results summarized in FIGS. 7G to 7J suggest that RRx-001 treatment reduces DSS-induced expression of colon homogenate IL-1β.

### Intraperitoneal Injection

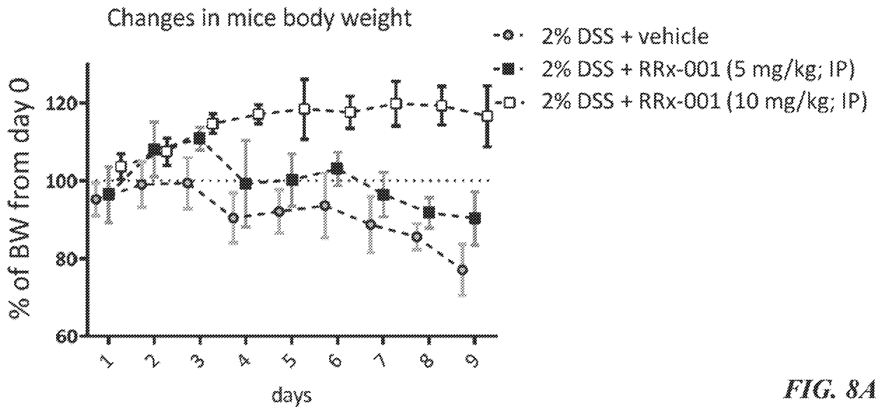

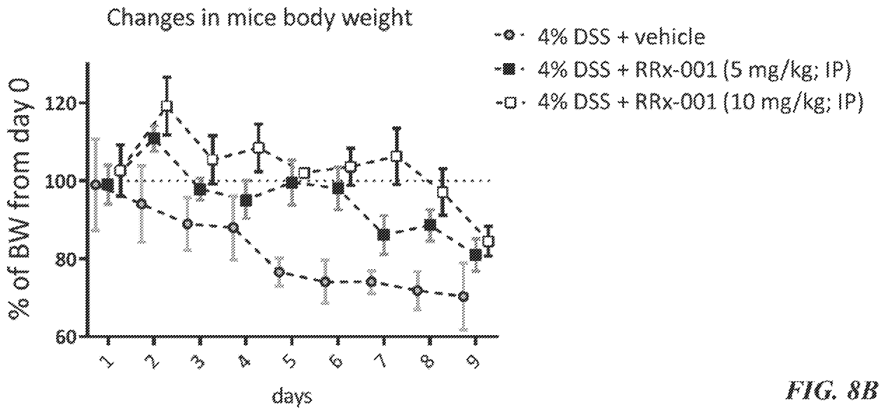

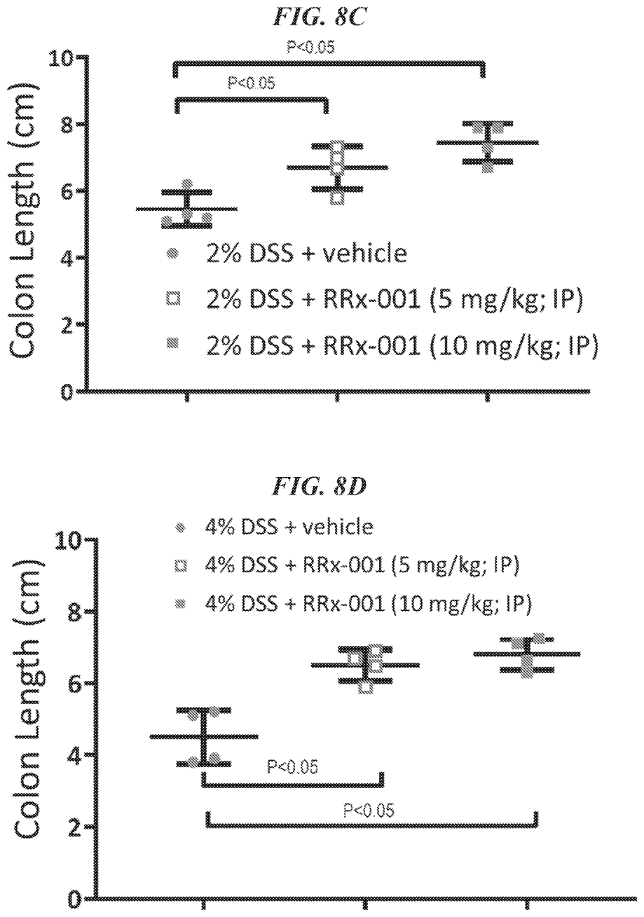

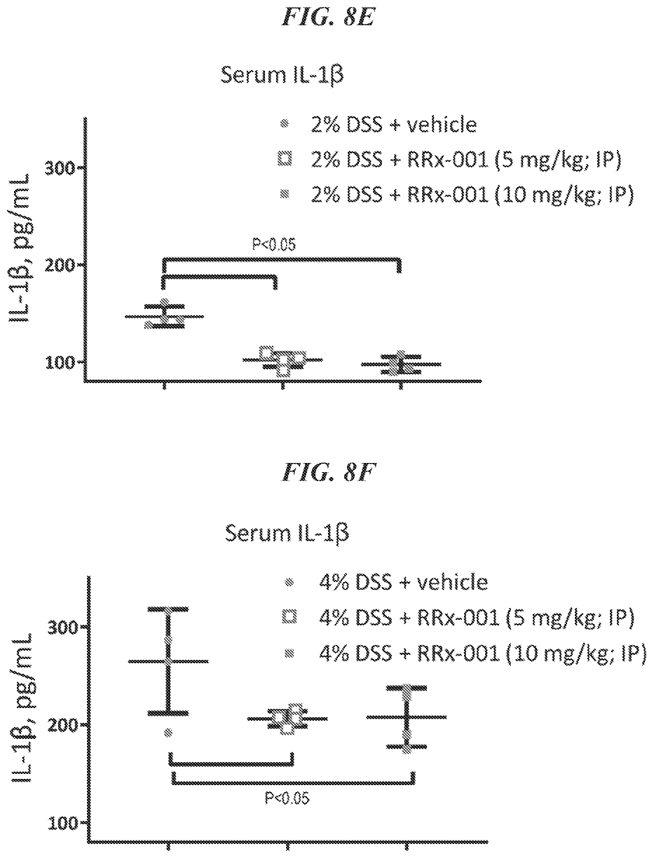

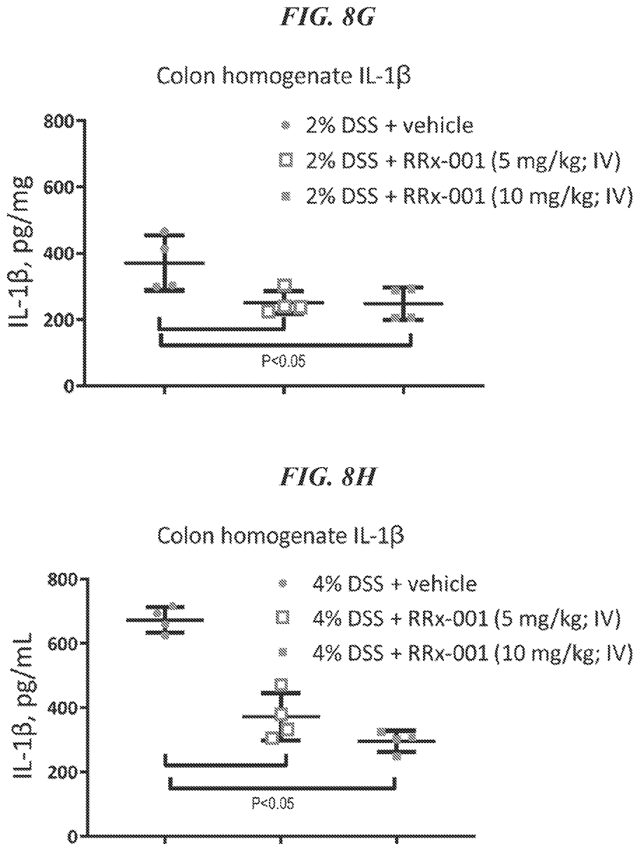

FIGS. 8A and 8B depict the changes in mice body weight over the 9-day period for each of the six groups: (i) 2% DSS+vehicle. (ii) 2% DSS+RRx-001 (5 mg/kg), and (iii) 2% DSS+RRx-001 (10 mg/kg) in FIG. 8A; and (iv) 4% DSS+vehicle, (v) 4% DSS+RRx-001 (5 mg/kg), and (vi) 4% DSS+RRx-001 (10 mg/kg) in FIG. 8B. While the 2% or 4% DSS was given ad libitum for 9 days as described above, vehicle, 5 mg/kg RRx-001, or 10 mg/kg RRx-001 was given as intraperitoneal injections every other day during the 9-day period. The results suggest that RRx-001 treatment significantly mitigates body weight loss in mice due to DSS induced colitis. FIGS. 8C and 8D depict colon length of mice in each of the six groups. The results suggest that RRx-001 treatment mitigates the colon length reduction due to DSS induced colitis. FIGS. 8E and 8F depict serum IL-1β levels in mice in each of the six groups. The results suggest that RRx-001 treatment reduces DSS-induced expression of serum IL-1β. FIGS. 8G and 8H depict colon homogenate IL-1β levels in mice in each of the six groups. The results suggest that RRx-001 treatment reduces DSS-induced expression of colon homogenate IL-1β.

### INCORPORATION BY REFERENCE

The entire disclosure of each of the patent documents and scientific articles referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

1. A method of treating a disorder selected from the group consisting of an autoimmune disorder, inflammatory disorder, neurodegenerative disorder, and neuromuscular disorder in a subject in need thereof, the method comprising administering a loading dose of RRx-001 or an analog thereof to the subject in an amount effective to ameliorate a symptom of the disorder, and thereafter administering a maintenance dose of RRx-001 or the analog thereof to maintain the amelioration of the symptom for a prolonged period of time.

2. The method of claim 1, wherein the loading dose comprises at least 1 mg (e.g., 1.5 mg, 2 mg, 2.5 mg or 3 mg) on a first day.

3. The method of claim 1, wherein the loading dose comprises at least 1 mg/m² (e.g., 1.5 mg/m², 2 mg/m², 2.5 mg/m², or 3 mg/m²) on a first day.

4. The method of claim 2 or 3, wherein the loading dose comprises multiple doses administered after the first day with a periodic interval of at least 3, 4, 5, 6, 7, 14, 21, or 28 days separating each dose so as to achieve amelioration of the symptom on the disorder.

5. The method of claim 4, wherein the doses administered after the first day are the same as the dose administered on the first day.

6. The method of any one of claims 1-5, wherein the maintenance dose is administered after the loading dose with a periodic interval of about 7 days, about 14 days, about 21 days, or about 28 days separating each dose so as to maintain amelioration of the symptom of the disorder.

7. The method of any one of claims 1-5, wherein the maintenance dose is administered after the loading dose with a periodic interval of 7 days, 14 days, 21 days, or 28 days separating each dose so as to maintain amelioration of the symptom of the disorder.

8. The method of any one of claims 1-7, wherein the maintenance dose is less than the loading dose.

9. The method of any one of claims 1-8, wherein the prolonged period of time is at least 1 month, or 3, 6, 9, or 12 months.

10. The method of any one of claims 1-9, wherein administering the loading dose and administering the maintenance dose does not lead to toxic systemic side effects.

11. The method of claim 10, wherein the toxic systemic side effects are selected from the group consisting of dyspnea, cough, fatigue, cardiac symptoms, electrolyte deficiencies, thyroid dysfunction, bone loss, sleep issues, nephrotoxicity, neurologic symptoms, autoimmunity, retinitis, hepatotoxicity, gastrointestinal distress, weight loss, malaise, rash, and leukopenia with increased risk of infection and the development of malignancy, and combinations thereof.

12. A method for increasing compliance and tolerability in a subject in need of treatment for an autoimmune disorder, inflammatory disorder, neurodegenerative disorder, or neuromuscular disorder, the method comprising administering a therapeutically effective amount of RRx-001 or an analog thereof; wherein administration of the therapeutically effective amount does not cause hematologic, neurologic, pulmonary, metabolic, cardiovascular, dermatologic, nephrologic, gastrointestinal, genitourinary, inflammatory, autoimmune, thyroidal, and immunodeficiency-related side effects, and wherein the subject completes treatment with a cumulative dose of at least 1 mg or 1 mg/m² of RRx-001 or an analog thereof.

13. A method of preventing the initiation, development or worsening of a symptom of a disorder selected from the group consisting of an autoimmune disorder, inflammatory disorder, neurodegenerative disorder, and neuromuscular disorder in a subject in need thereof, the method comprising administering an effective amount of RRx-001 or an analog thereof to the subject to prevent the initiation, development or worsening of the symptom of the disorder.

14. The method of claim 13, wherein the effective amount comprises at least 1 mg (e.g., 1.5 mg, 2 mg, 2.5 mg or 3 mg).

15. The method of claim 13, wherein the effective amount comprises at least 1 mg/m² (e.g., 1.5 mg/m², 2 mg/m², 2.5 mg/m², or 3 mg/m²).

16. The method of claim 14 or 15, wherein the RRx-001 or the analog thereof is administered to the subject periodically (e.g., once every week, two weeks, or three weeks, or 4 weeks, or once a month, two months, three months or four months).

17. The method of any one of claims 1-16, wherein the disorder is an autoimmune disorder.

18. The method of any one of claims 1-16, wherein the disorder is a metabolic disorder.

19. The method of any one of claims 1-16, wherein the disorder is a bone or joint disorder.

20. The method of claim 17, wherein the autoimmune disorder is selected from the group consisting of rheumatoid arthritis (RA), sarcoidosis, systemic lupus erythematosus (SLE), Huntington's disease, end stage renal disease, systemic sclerosis or scleroderma, myositis, diabetes type 1, multiple sclerosis, Sjögren's syndrome, psoriasis, primary biliary cirrhosis, autoimmune hepatitis, Graves' disease, Addison's disease, tuberculosis, Crohn's disease, ulcerative colitis, inflammatory bowel disease, autoimmune hemolytic anemia, idiopathic thrombocytopenic purpura, idiopathic neutropenia and coeliac disease.

21. The method of any one of claims 1-16, wherein the disorder is an inflammatory disorder.

22. The method of claim 21, wherein the inflammatory disorder is selected from the group consisting of sarcoidosis, vitiligo, polymyalgia rheumatica, graft vs. host disease (GvHD) or an autoimmune reaction after organ transplantation, Churg-Strauss syndrome, chronic gastritis, pernicious anemia, lichen sclerosis, long COVID, Huntington's disease, multiple sclerosis, asthma, Wegener's granulomatosis, autoimmune enteropathy, PANDAS, rheumatic fever, dermatomyositis, Goodpasture syndrome, primary biliary cirrhosis, diabetes type 1, autoimmune hepatitis, Graves' disease, Crohn's disease, ulcerative colitis, coeliac disease, Addison's disease, Sjogren's syndrome, systemic lupus erythematosus and rheumatoid arthritis.

23. The method of claim 18, wherein the metabolic disorder is selected from the group consisting of gout, type 2 diabetes, atherosclerosis, fatty liver disease, stroke and cardiovascular disease.

24. The method of claim 19, wherein the bone and joint disorder is selected from the group consisting of osteomyelitis, infectious arthritis, sarcoidosis, rheumatoid arthritis, ankylosing spondylitis, psoriatic arthritis, Lyme arthritis and osteoarthritis.

25. The method of any one of claims 1-16, wherein the disorder is a neurodegenerative disorder.

26. The method of claim 25, wherein the neurodegenerative disorder is selected from the group consisting of Alzheimer's disease and other dementias, Parkinson's disease (PD) and PD-related disorders, multiple system atrophy, prion diseases, motor neuron diseases (MND), Huntington's disease, spinocerebellar ataxia, spinal muscular atrophy, Batten disease, Freidreich's ataxia, vascular dementia, transactive response DNA-binding protein-43, proteinopathies, incurable neurodegenerative diseases with pediatric onset, purine and pyrimidine defects, metal metabolism such as Wilson disease, pantothenate kinase associated neurodegeneration and neurodegeneration with brain iron accumulation, leukodystrophy, peroxisomal disorders, lysosomal storage disorders, congenital disorders of glycosylation, creatine disorders, and Rasmussen's encephalitis.

27. The method of any one of claims 1-16, wherein the disorder is a neuromuscular disorder.

28. The method of claim 27, wherein the neuromuscular disorder is selected from the group consisting of' amyotrophic lateral sclerosis, multiple sclerosis, myasthenia gravis, Charcot-Marie-Tooth disease and related hereditary neuropathies, chronic inflammatory demyelination polyneuropathy, Guillain-Barre syndrome, Lambert Eaton syndrome, Miller-Fisher, muscular dystrophies, myopathies, peripheral neuropathies, neurotransmitter defects, immunoglobulin M gammopathy-associated neuropathy paraneoplastic neurological syndrome, and stiff man syndrome.

29. A method of treating an autoimmune disorder selected from the group consisting of rheumatoid arthritis (RA), sarcoidosis, systemic lupus erythematosus (SLE), Huntington's disease, end stage renal disease, systemic sclerosis or scleroderma, myositis, diabetes type 1, multiple sclerosis, Sjögren's syndrome, psoriasis, primary biliary cirrhosis, autoimmune hepatitis, Graves' disease, Addison's disease, tuberculosis, Crohn's disease, ulcerative colitis, inflammatory bowel disease, autoimmune hemolytic anemia, idiopathic thrombocytopenic purpura, idiopathic neutropenia and coeliac disease in a subject in need thereof, the method comprising administering an effective amount of RRx-001 or an analog thereof to ameliorate a symptom of the disorder.

30. A method of treating an inflammatory disorder selected from the group consisting of sarcoidosis, vitiligo, polymyalgia rheumatica, graft vs. host disease (GvHD) or an autoimmune reaction after organ transplantation, Churg-Strauss syndrome, chronic gastritis, pernicious anemia, lichen sclerosis, long COVID, Huntington's disease, multiple sclerosis, asthma, Wegener's granulomatosis, autoimmune enteropathy, PANDAS, rheumatic fever, dermatomyositis, Goodpasture syndrome, primary biliary cirrhosis, diabetes type 1, autoimmune hepatitis, Graves' disease, Crohn's disease, ulcerative colitis, coeliac disease, Addison's disease, Sjogren's syndrome, systemic lupus erythematosus and rheumatoid arthritis in a subject in need thereof, the method comprising administering an effective amount of RRx-001 or an analog thereof to ameliorate a symptom of the disorder.

31. A method of treating a neurodegenerative disorder selected from the group consisting of Alzheimer's disease and other dementias, Parkinson's disease and PD-related disorders, prion diseases, motor neuron diseases (MND), Huntington's disease, spinocerebellar ataxia, spinal muscular atrophy, Batten disease, Freidreich's ataxia, vascular dementia, multiple system atrophy, transactive response DNA-binding protein-43, proteinopathies, incurable neurodegenerative diseases with pediatric onset, purine and pyrimidine defects, metal metabolism such as Wilson disease, pantothenate kinase associated neurodegeneration and neurodegeneration with brain iron accumulation, leukodystrophy, peroxisomal disorders, lysosomal storage disorders, congenital disorders of glycosylation, creatine disorders, and Rasmussen's encephalitis in a subject in need thereof, the method comprising administering an effective amount of RRx-001 or an analog thereof to ameliorate a symptom of the disorder.

32. A method of treating Alzheimer's disease, the method comprising administering an effective amount of RRx-001 or an analog thereof to ameliorate a symptom of Alzheimer's disease.

33. A method of treating Parkinson's disease, the method comprising administering an effective amount of RRx-001 or an analog thereof to ameliorate a symptom of Parkinson's disease.

34. A method of treating a neuromuscular disorder selected from the group consisting of amyotrophic lateral sclerosis, multiple sclerosis, myasthenia gravis, Charcot-Marie-Tooth disease and related hereditary neuropathies, chronic inflammatory demyelination polyneuropathy, Guillain-Barre syndrome, Lambert Eaton syndrome, Miller-Fisher, muscular dystrophies, myopathies, peripheral neuropathies, neurotransmitter defects, immunoglobulin M gammopathy-associated neuropathy paraneoplastic neurological syndrome, and stiff man syndrome in a subject in need thereof, the method comprising administering an effective amount of RRx-001 or an analog thereof to ameliorate a symptom of the disorder.

35. A method of treating a disorder or condition associated with interleukin 1 beta (IL-1β) or interleukin 18 (IL-18) expression or release, the method comprising administering an effective amount of RRx-001 or an analog thereof to ameliorate a symptom of the disorder or condition.

36. The method of claim 35, wherein the disorder or condition is an autoimmune disorder or an inflammatory disorder.

37. The method of claim 35, wherein the disorder or condition is pediatric fever syndrome, acne vulgaris, hidradenitis suppurativa, psoriasis, rheumatoid, systemic juvenile idiopathic arthritis sepsis, chronic inflammation, aging, viral infection, asthma, congestive heart failure, angina, liver steatosis, diabetes, familial cold autoinflammatory syndrome (FCAS), Muckle-Wells syndrome (MWS), neonatal-onset multisystem inflammatory disease (NOMID), chronic infantile neurologic, cutaneous, arthritis (CINCA) syndrome, or cerebral edema due to intracerebral hemorrhage.

38. The method of any one of claims 35-37, wherein administering an effective amount of RRx-001 or an analog thereof does not lead to severe infection or end organ toxicity.

39. The method of any one of claims 35-38, wherein the method further comprises administering a second agent.

40. The method of claim 39, wherein the second agent comprises an IL-1α inhibitor, an IL-1β inhibitor, an IL-18 inhibitor, or an inflammasome inhibitor, or any combination thereof.

41. The method of claim 39 or 40, wherein the second agent comprises canakinumab, anakinra, rilonacept, resveratrol, curcumin, MABp1, GSK-1070806 antibody, MCC950, β-hydroxybutyrate, glibenclamide, oridonin, JC121, JC124, YQ128, apigenin, cardamonin, Isoliquiritigenin, Glycyrrhizin, Luteolin, Quercetin, Artemisia, Caffeic acid phenethyl ester, Obovatol, Parthenolide, Shikonin, Sulforaphane, Tranilast, OLT1177 (Dapansutrile), CY-09, Methylene Blue, Disulfuram (Antabuse), Fenamic acid derivatives, Fluoxetine, β-Hydroxybutyrate (BHB), BAY 11-7082 (BAY), or X-11-5-27, or any combination thereof.

42. The method of any one of claims 39-41, wherein a side effect associated with the second agent is reduced.

43. A method of controlling activity of a mononuclear phagocyte, the method comprising exposing the mononuclear phagocyte to an effective amount of RRx-001 or an analog thereof.

44. The method of claim 43, wherein the activity of the mononuclear phagocyte is expression or release of interleukin 1 beta (IL-1β) from the mononuclear phagocyte.

45. The method of claim 43 or 44, wherein controlling activity of the mononuclear phagocyte is reducing the expression or release of interleukin 1 beta (IL-1β) from the mononuclear phagocyte.

46. The method of any one of claims 43-45, wherein the mononuclear phagocyte is a macrophage or a dendritic cell, or a combination thereof.

47. The method of any one of claims 43-46, wherein the exposure occurs ex vivo.

48. The method of any one of claims 43-47, wherein the mononuclear phagocyte is collected from a human subject.

49. The method of claim 48, wherein the mononuclear phagocyte exposed to the effective amount of RRx-001 or an analog thereof is re-introduced into the human subject.

50. The method of claim 49, wherein the mononuclear phagocyte exposed to the effective amount of RRx-001 or an analog thereof is re-introduced into the human subject by infusion.

51. The method of claim 49 or 50, wherein immunological tolerance is increased in the human subject after the mononuclear phagocyte is re-introduced.

52. The method of any one of claims 29-51, wherein the method comprises the dosing regimen of any one of claims 1-9.

53. The method of any one of claims 1-52, wherein the RRx-001 or analog thereof is combined with blood prior to administration to the subject.

54. The method of claim 53, wherein the blood is harvested from the subject.

55. The method of any one of claims 1-34 and 52-54, further comprising administering a second, different agent to the subject.

56. The method of any one of claims 1-34 and 52-55, wherein the subject is a human.

57. The method of any one of claims 1-56, wherein the RRx-001 or analog thereof reduces the toxicity of a co-administered or subsequently administered NSAID, corticosteroid, DMARD or biologic DMARD; wherein the toxicity is hematologic, neurologic, pulmonary, metabolic, cardiovascular, dermatologic, nephrologic, gastrointestinal-, genitourinary-, inflammatory, autoimmune-related, thyroidal and/or bone marrow-related; and wherein the subject has an autoimmune, inflammatory, neuromuscular or neurodegenerative disorder.

58. The method of any one of claims 1-57, wherein the RRx-001 or analog thereof is administered orally.

59. The method of any of claims 1-58, wherein the RRx-001 or analog thereof is administered in a swish and spit and/or swish and swallow formulation.

60. The method of any of claims 1-57, wherein the RRx-001 or analog thereof is administered sublingually.

61. The method of any of claims 1-57, wherein the RRx-001 or analog thereof is administered subcutaneously.

62. A pharmaceutical composition comprising RRx-001 or an analog thereof and at least one additional active component selected from the group consisting of an anti-inflammatory agent, a neuroprotective agent, a corticosteroid, an immunosuppressant, and a disease-modifying anti-rheumatic drug (DMARD), and one or more pharmaceutically acceptable excipients.
